(19) United States
(12) Patent Application Publication
(10) Pub. No.: US 2017/0119806 A1
(43) Pub. Date: May 4, 2017

(54) ARYLNAPHTHALENE LACTONE DERIVATIVES AND METHODS OF MAKING AND USING THEREOF

(71) Applicant: OHIO STATE INNOVATION FOUNDATION, Columbus, OH (US)

(72) Inventors: Alan Douglas Kinghorn, Columbus, OH (US); Yulin Ren, Upper Arlington, OH (US); Heebyung Chai, Columbus, OH (US); James Robert Fuchs, Columbus, OH (US); Jack Charles Yalowich, Bexley, OH (US); Jianhua Yu, Columbus, OH (US); John Woodward, Columbus, OH (US)

(21) Appl. No.: 15/301,328

(22) PCT Filed: Mar. 31, 2015
(86) PCT No.: PCT/US15/23657

§ 371 Date: Sep. 30, 2016

Related U.S. Application Data
(60) Provisional application No. 61/972,958, filed on Mar. 31, 2014.

(51) Int. Cl. A61K 31/7048; A61K 31/365; A61N 5/10; A61K 45/06

(52) U.S. Cl. CPC A61K 31/7048; A61K 45/06; A61K 31/365; A61N 5/10

(57) ABSTRACT

A series of natural products including phyllanthusum, an arylnaphthalene lignan derivative, with anticancer and antitumor and immunostimulating activity are disclosed. The invention further encompasses methods of adding water solubilizing groups to the arylrings that include phosphonyl groups.

### STATEMENT OF GOVERNMENT SUPPORT

This invention was made with government support under Grant No, CA125066, Grant No. CA090787, Grant No. CA155521, Grant No, OD018403, Grant No. CA163205, and Grant No. CA068458, all awarded by the National Institutes of Health. The government has certain rights in the invention.

### BACKGROUND

Natural products and their semi-synthetic derivatives are used widely in cancer chemotherapy (Newman D J and Cragg G M. J. Nat. Prod. 2012, 75, 311-335; Kinghorn A D et al, Pure Appl. Chem, 2009, 81, 1051-1063). As an example, etoposide (VP-16) is a semi-synthetic aryltetratin lignan glycoside modeled on the natural product podophyllotoxin. It targets DNA topoisomerase II (topo II) and has been utilized for decades to treat several types of cancer (Meresse P et al. Curr. Med Chem. 2004, 11, 2443-2466). However, side effects have been reported for etoposide, including myelosuppression and the development of secondary leukemias linked to topo II inhibitory activity (Ezoe S. Int. J. Environ. Res. Public Health 2012, 9, 2444-2453).

Podophyllotoxin is an aryltetralin ligan that occurs in Podophyllum peltatum and P. emodi var. hexandrum (syn. Sinopodophyllum hexandrum) (Berberidaceae) (Meresse P et al. Curr. Med Chem, 2004, 11, 2443-2466; Chattopadhyay S et al. Nat. Prod Res. 2004, 18, 51-57; Girl A and Narasu M L. Cytotechnoiogy 2000, 34, 17-26). In addition to Podophyllum species (Atta-ur-Rahman et al. Phytochemistry 1995, 40, 427-43 a number of arylnaphthalene lignan lactones, structurally similar to podophyllotoxin, have been identified as minor constituents from plants in the genera Cleistanthus (Euphorbiaceae) (Pinho P M M and Kijioa A Phytochem. Rev, 2007, 6, 175-182), Haplophyllum (Rutaceae) (Oozier B et al. Phytochemistry 1996, 42, 689-693; Al-Abed Y et al. Phytochemistry 1998, 49, 1779-1781), Justicia (Acanthaceae) (Susplugas S et al. J. Nat. Prod, 2005, 68, 734-738), Mananthes (Acanthaceae) (Tian J et al. Helv. Chim. Acta 2006, 89, 291-298), and Phyllanthus (Phyllanthaceae) (Lin M T et al. J. Nat, Prod. 1995, 58, 244-249; Tuchinda P et al. Planta Med, 2006, 72,60-62; Wu S J and Wu T S. Chem. Pharm. Bull. 2006, 54, 1223-1225; Tuchinda P et al. J. Nat. Prod 2008, 71, 655-663; Wang C Y et al. Phytochem. Anal. 2011, 22, 352-360). .Many naturally occurring arylnaphthalene lignan lactones have been reported to possess cytotoxicity toward panels of human cancer cell lines (Susplugas S et al. J. Nat. Prod. 2005, 68, 734-738; Lin M T et al. J. Nat. Prod. 1995, 58, 244-249; Tuchinda P et al. Planta Med. 2006, 72, 60-62; Wu S J and Wu T S. Chem. Pharm. Bull. 2006, 54, 1223-1225; Tuchinda P et al. J. Nat. Prod 2008, 71, 655-663; Wang C Y et al. Phytochem. Anal. 2011, 22, 352-360; Fukarniya N and Lee K H. J. Nat. Prod. 1986, 49, 348-350; Novelo M et al. J. Nat. Prod. 1993, 56, 1728-1736; Day S H et al, J. Nat. Prod. 1999, 62, 1056-1058; Innocenti G et al. Chem. Pharm. Bull. 2002, 50, 844-846; Day S H et al. J. Nat. Prod 2002, 65, 379-381; Ramesh C et al. Chem. Pharm. Bull. 2003, 51, 1299-1300; Vasilev N et al. J Nat. Prod. 2006, 69, 1014-1017), and several of their synthetic analogues also showed such activity (Zhao Y et al. Arch. Pharm. Chem, Life Sci. 2012, 345, 622-628; Shi D K et al. Eur. J. Med. Chem. 2012, 47, 424-431). Some arylnaphthalene lignin lactones have exhibited in vivo antitumor efficacy (Rezanka T etal. Phytochemistry 2009, 70, 1049-1054; Kang K et al. Neoplasia 2011, 13, 1043-1057), and a compound, cleistanthin B, showed selective cytotoxicity toward human tumor cells (Kumar C P P et al. Mutagenesis 1996, 11, 553-557). Some of these compounds showed a mechanism of action different from etoposide (Susplugas S et al. J. Nat. Prod. 2005, 68, 734-738; Kang K et al. Neoplasia 2011, 13, 1043-1057), and several analogues did not act as topo II poisons mechanistically (Zhao Y et al. Arch. Pharm. Chem. Life Sci. 2012, 345, 622-628; Shi D K et al. Eur. J. Med. Chem. 2012, 47, 424-431). What are needed are new compositions for the treatment of cancer, e.g., arylnaphthalene intone derivatives. The compounds, compositions and methods disclosed herein address these and other needs.

### SUMMARY

In accordance with the purposes of the disclosed materials, compounds, compositions, kits and methods, as embodied and broadly described herein, the disclosed subject matter relates to compounds, compositions, methods of making said compounds and/or compositions, and methods of using said compounds and/or compositions. More specifically, arylnaphthalene lactone derivatives are provided herein. Also disclosed herein are methods of use of the disclosed arylnaphthalene lactone derivatives as anticancer and immunostimulant agents.

Additional advantages will be set forth in part in the description that follows or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of specification, illustrate several aspects described below.

FIG. 1 displays the structures of several arylnaphthalene lignans.

FIG. 2 displays the COSY (-) and key HMBC (→) NMR correlations of compounds 2-8.

FIG. 3 displays selected NOESY ( [symbol] , ¹H→¹H) correlations of compounds 2-5 and 7,

FIG. 4 displays COSY (-, ¹H→¹H), key HMBC (→, ¹H→¹³C), and selected NOESY (⇄, ¹H→¹H) correlations of (phyllanthusmin D).

FIG. 5 displays the effect of phyllanthusmin D (1) on the growth of human colon cancer HT-29 cells implanted in NCr nu/nu mice tested by an in vivo hollow fiber assay. Mice were treated with the indicated doses of 1 once a day by intraperitoneal injection from day 3 to day 6 after implantation of the HT-29 cells facilitated in hollow fibers. On day 7, mice were sacrificed, and fibers were retrieved and analyzed, The results are shown as the average percent cell growth relative to control. Columns: mean in each group (n=6 for the control group and n=3 for the treatment group); bars, SE; **p≦0.05 and ***p≦0.01 for significant differences from the 5 mg/kg (1) treatment.

FIG. 6 displays the evaluation of arylnaphthalene fignans phyllanthusmin C (4), phyllanthusmin D (1), and 7-O-((2,3,4-tri-O-acetyl)-α-L-arabinopyranosyl) diphyllin (7) from Phyllanthus poilanei for activity as topoisomerase IIα (topo IIα) inhibitors. Topo II-DNA covalent complexes induced by test samples and etoposide with sodium dodecyl sulfate (SDS), digesting away the enzyme, and releasing the cleaved DNA as linear DNA. The formation of linear DNA was detected by separating the SDS-treated reaction products using ethidium bromide gel electrophoresis and quantified by accounting for the relationship between fluorescence and relative band intensity for open circular (OC), linear (LNR), supercoiled (SC), and relaxed (RLX) configurations of DNA.

FIG. 7 displays HT-29 cell apoptosis induction of phyllanthusmin D (1) and etoposide. HT-29 cells were treated with 1 μM or 5 μM phyllanthusmin D (1), 1 μM or 5 μM etoposide, to the vehicle control for 72 hours, Rillowed by an Annexin V staining method. Lower left quadrant: the percentage of viable cells; lower right quadrant: the percentage of apoptotic cells; upper left quadrant: the percentage of necrotic cells; upper right quadrant: the percentage of the late-stage apoptotic or dead cells.

FIG. 8 displays caspase-3 activation by 1 in HT-29 cells. HT-29 cells were incubated with phyllanthusmin D (1) and etoposide with different concentrations for 24 hours, and caspase-3-like activity was determined by western blot using rabbit monoclonal cleaved caspase-3 (Asp175) antibody. The data shown are a representative blot from three independent experiments with similar results.

FIG. 9 displays a schematic of a convergent synthesis of phyllanthusmins through tate-stage glycosylation of the diphyilin core.

FIG. 10 displays a schematic of the synthesis of the diphyllin core.

FIG. 11 displays a schematic of glycosylation of the diphyilin core.

FIG. 12 displays the phyllanthusmin analogues evaluated in vitro.

FIG. 13 displays a schematic of the synthesis of compound PHY-9.

FIG. 14 displays a schematic of the synthesis of compounds PHY-6 and PHY-8.

FIG. 15 displays the antiproliferative activity of various phenols against HT-29 cells.

FIG. 16 displays a schematic of the synthesis of compound PHY-14.

FIG. 17 displays differentially functionalized diphyllin lignan arabinoses.

FIG. 18 displays a series of analogues.

FIG. 19 displays that PL-C (phyllanthusmin C, 4) can enhance IFN-γ production in human primary NK cells. (A) Chemical structure of PL-C (phyllanthusmin C, 4), (9) Healthy donor PBMCs (left panel) or enriched NK cells (right panel) were treated with DMSO vehicle control or 10 μM PL-C for 18 h in the presence of 2 (10 ng/mL) or IL-15 (100 ng/mL). The cells were harvested and analyzed by intracellular flow cytometry to determine the frequency of IFN-γ⁺ cells in CD56⁺CD3⁻ NK cells (n=8 for PBMC and n=5 for enriched NK). (C) Highly purified (≧99.5%) human primary NK cells were treated with 10 μM PL-C for 18 h to determine the levels of IFN-γ secretion by ELISA. IFN-γ secretion from treatment with PL-C alone (left panel) or in combination with IL-12 (10 ng/mL, middle panel) or IL-15 (100 ng/mL, right panel) is shown. (D) Cells were treated as described in (C) and harvested at 12 h, IFNG mRNA expression was assessed by real-time RT-PCR, and the relative IFNG mRNA expression of each treatment was normalized to untreated vehicle control in the same donor. Data are shown as mean±SEM (n=6 in each treatment; error bars represent SEM). *p<0.05, **p<0.01, which denote statistical comparison between the two marked treatment groups (B-D). (E) Highly purified (≧99,5%) primary human NK cells were treated with 10 μM. PL-C in combination with various concentrations of IL-12 (10, 1, and 0.1 ng/mL) or IL-15 (100, 10, and 1 ng/mL) for 24 h to determine the levels of IFN-γ secretion. Representative data from one of three donors with the similar data are shown. *p<0.05, **p<0.01, which denote statistical comparison between the two marked treatment groups and are calculated from data of all tested donors. Error bars represent SD. (F) NKL cells were treated with 10 μM PL-C in the presence of IL-12 or IL-15 for 18 or 12 h to determine the levels of IFN-γ secretion (left panel) or IFNG mRNA expression (right panel), respectively. Data shown represent at least three independent experiments. *p<0.05, **p<0.01, respectively, compared with vehicle control Error bars represent SD.

FIG. 20 displays (A) photographs of representative source plants, Phyllanthus reticulatus (left) and Phyllanthus poilanei (right). (B) Purified primary human NK cells were treated as described in FIG. 37C. The increase of IFN-γ in each case is presented as percent increase above treatment with vehicle control [untreated with PL-C (phyllanthusmin C, 4) or cytokines]. In each donor, the paired bars compare the additive effect of IL-12 and PL-C-treated alone (left, composite bar) versus the effect of the co-stimulation with IL-12 and PL-C (right, black bar). Representative data of 3 out of 14 donors are shown. p<0.001, additive effect of IL-12 and PL-C versus co-stimulation with IL-12 and PL-C.

FIG. 21 displays PL-C (phyllanthusmin C, 4) does not affect T IFN-γ production and primary NK cytotoxic activity. (A) Human RBMCs were treated with DMSO vehicle control or 10 μM PL-C for 18 hours in the presence of IL-12 (10 ng/mL) or IL-15 (100 ng/mL), as described in FIG. 37C. The cells were harvested for intracellular flow cytometry to determine the frequency of IFN-γ⁺ cells in CD56⁻CD3⁺CD4⁻ or CD56⁻CD3⁺CD8⁺T. Representative data from 1 out of 5 donors are shown. (B) Purified primary NK cells were treated with IL-12 (10 ng/mL) or IL-15 (100 ng/mL) with or without 10 μM PL-C for 8 hours and were sequentially co-cultured with ^{5I}Cr labeled ARH-77 cells at various effector/target cell ratios for additional 4 hours. ⁵¹Cr release was measured by a TopCount counter. Data shown are the means of 3 donors. There is no statistically significant difference between vehicle control and PL-C treatment group in all conditions. Error bars represent S.D. (C) Purified primary NK cells were treated with 10 μM PL-C in the presence of IL-12 (10 ng/mL) (top) or IL-15 (100 ng/mL) (bottom) for 12 hours, and cell pellets were harvested for detecting granzyme A (GZMA), granzyme B (GZMB), perforin (PRF1) and Fas (Fasl) mRNA expression level by real-time RT-PCR. Data shown are the means of 6 donors. p>0.05, vehicle control versus PL-C in all panels. Error bars represent S.D.

FIG. 22 displays that PL-C (phyllanthusmin C, 4) can activate both CD56^{dim} and CD56^{bright} NK cells to secrete IFN-γ. (A) Enriched NK cells were sorted via FACS into CD56^{dim} and CD56^{bright} NK cells, based on the relative density of CD56 expressed on the cell surface. CD56^{dim} and CD56^{bright} NK cells were treated with 10 μM PL-C in the presence of IL-12 (10 ng/mL) for 18 hand assessed for the levels of IFN-γ secretion by ELISA. (B) Cells were isolated, treated, and analyzed as in (A) but in the presence of IL-15 (100 ng/mL) instead of IL-12. Representative data from one of at least three donors with similar results are shown. *p<0.05, **p<0.01, which denote statistical comparison between the two marked treatment groups and are calculated from data of all tested donors (A and B). Error bars represent SD.

FIG. 23 displays that PL-C (phyllanthusmin C, 4) can increase the phosphorylation of p65 in human primary NK and NKL cells. (A) Purified primary human NK cells were treated with 5 and 10 μM PL-C for 18 h. The cells were harvested and lysed for immunoblotting using p65 and p-p65 Abs. β-Actin immunoblotting was included as the internal control. Data shown are for treatment with PL-C alone (top panel) or in combination with IL-12 (10 ng/mL) (middle panel) or IL-15 (100 ng/mL) (bottom panel) and are the representative plots of four donors with similar results. Numbers under each lane represent quantification of p-p65 or p65 via densitometry, after normalizing to β-actin. (B) NKL cells were treated, and data are presented as described in (A). Data from one of three independent experiments with similar results are shown. (C) Purified primary human NK (left panel) or NKL cells (right panel) were cotreated with 10 μM PL-C and IL-12 (10 ng/mL) in the presence or absence of the NF-κB inhibitor TPCK (10 μM) for 18 h. Supernatants were assayed for IFN-γ secretion by ELISA (top panel), and cells were harvested and lysed for immunoblotting of p-p65 (bottom panel). Representative data from one of three donors with the similar data (left panel) and the summary of three independent experiments with similar results (right panel) are shown. **p<0.01. Error bars represent SD.

FIG. 24 displays the effcts of PL-C (phyllanthustain C, 4) on IL-12 and IL-15 signaling pathways. (A) Purified human primary NK cells were treated with 10 μM PL-C in the presence of IL-2 (10 ng/mL) or IL-15 (100 ng/mL) for 12 hours. DMSO-treated cells served as vehicle controls. Cell pellets were harvested to extract total RNA for real-time RT-PCR to determine mRINA expression levels of IL-12Rβ1, IL-12Rβ2, IL-15Rα and IL-15β. Data are shown as means of 3 donors, * and ** indicate p<0.05 and p<0.01, respectively, which denote a statistical comparison between the two marked treatment groups. Error bars represent S.D. (B, C) Purified primary NK cells (B) or NKL cells (C) were treated with 5 or 10 μM PL-C in the presence of IL-12 (10 ng/mL) or IL-15 (100 ng/mL) for 4 hours. DMSO-treated cells served as vehicle controls. Cell pellets were harvested for subsequent immunoblotting of T-BET, phosphorylated STAT3 (p-STAT3), p-STAT4, p-STAT5, STAT3, STAT4, and STAT5. Data represent 1 out of 3 donors with similar data (B) and 3 independent experiments with similar results (C). Numbers underneath each lane represent quantification of detected protein by densitometry, after normalizing to β-actin.

FIG. 25 displays that PL-C (phylianthusmin C, 4) can augment the binding of p65 to the IFNG promoter in human NK cells. (A) Schematic of IFNG promoter potential binding sites for p65 (45). (B) NK cells purified from healthy donors were treated with 10 μM PL-C or DMSO vehicle control in the presence of IL-12 (10 ng/mL) for 12 h. Cell pellets were harvested for nuclear extraction, followed by EMSA with a ³²P-labeled oligonucleotide containing the C₃-3P NF-κB p65 binding site of the IFNG promoter. Data shown represent one of three donors with similar results. (C) Cells were treated as described in (B), and the cell pellets were harvested to extract protein for ChIP assay of p65 binding to the IFNG promoter locus C₃-3P. Mean of relative association of p65 at the IFNG promoter locus C₃-3P from three independent experiments is shown. *p<0.05, compared with cells treated with IL-12 alone. Error bars represent S.D.

FIG. 26 displays that TLR1 and/or TLR6 mediate IFN-γ induction by PL-C (phyllanthusmin C, 4) in human NK cells. (A) Human NK cells were purified and pretreated with a nonspecific IgG or anti-TLR1, anti-TLR3, anti-TLR6 or the combination of TLR1 and TLR6 blocking Abs (a) for 1 h. Cells were then treated with PL-C and IL-12 (10 ng/mL) for another 18 h, and supernatants were harvested to assess for IFN-γ secretion by ELISA (top panel) and cell pellets for p-p65 immunoblotting (bottom panel). Data shown are representative of one of six different donors with similar results. *p<0.05, **p<0.01, respectively, which denote statistical comparison between the two marked treatment groups and are calculated from data of all tested donors. Numbers underneath each lane represent quantification of protein by densitometry, normalized to β-actin, (B) Purified NK cells were treated with Pam₃CSK₄ (1 μg/mL; TLR1/2 ligand) or FSL-1 (1 μg/ml; TLR6/2 ligand) in the presence of IL-12 (10 ng/mL), with or without PL-C (10 μM) for 18 h, and then, supernatants were harvested to assay for IEN-γ, secretion by ELISA. Data shown are representative of one of six donors with similar results. *p<0.05, **p<0.01, which denote statistical comparison between the two marked treatment groups and are calculated from data of all tested donors. (C) Purified primary NK cells were treated with various low concentration of Pam₃CSK₄ or FSL-1 with or without PL-C (10 μM) in the presence of IL-12 (10 ng/mL), and then, supernatants were harvested to assay for IFN-γ secretion by ELISA. Data shown are representative one of three donors with similar results. Error bars indicate SD. (D) 293T cells were transfected with TLR1 (0.5 μg) or TLR6 (0.5 μg) expression plasmid along with pGL-3×κB-luc (1 μg) and pRL-TK renifia-luciferase control plasmids (5 ng; Promega). Cells were then treated with various concentration of PL-C for another 24 h with fresh medium, and DMSO was included as vehicle control. The ratio of the firefly to the renilla luciferase activities was used to show the relative luciferase activity, which corresponded to NY-κB activation. *p<0.05, **p<0.01, compared with vehicle control. Error bars represent SD. (E) NKL cells were infected with pSUPER or pSUPER-shTLR1 retroviruses and sorted based on GFP expression. After sorting, TLR1 mRNA knockdown was confirmed by reakime RT-PCR. (F) Both the vector-transduced cells (pSUPER) and the TLR1 knockdown (pSUPER-shTLR1) NKL cells were treated with or without PL-C in the presence or absence of IL-12 or IL-15. pellets were harvested at 12 h for real-time RT-PCR. The relative IFAIG mRNA expression induced by PL-C in the presence of IL-12 (10 ng/mL) or IL-15 (100 ng/mL) was shown in the upper or lower panel, respectively. The summary of three independent experiments with similar results are shown. **p<0.01. Error bars represent SD.

### DETAILED DESCRIPTION

The materials, compounds, compositions, articles, and methods described herein may be understood more readily by reference to the following detailed description of specific aspects of the disclosed subject matter and the Examples included therein.

Before the present materials, compounds, compositions, kits, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the fbliowing meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds, reference to “an agent” includes mixture of two or more such agents, and the like.

“Optional” or “optionally” means that the subsequently described event or circumstance can or cannot occur, and that the description includes instances where the event or circumstance occurs and instances where it does not.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

By “treat” or other fbrms of the word, such as “treated” or “treatment,” is meant to administer a composition or to perform a method in order to reduce, prevent, inhibit, or eliminate a particular characteristic or event (e.g., tumor growth or survival). The term “control” is used synonymously with the term “treat.”

The term “anticancer” refers to the ability to treat or control cellular proliferation and/or tumor growth at any concentration.

The term “therapeutically effective” means the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contac:t with the tissues of human beings and animals without excessive toxicity, irritation, allergic response, or other problems or complications commensurate with a reasonable benefit/risk ratio.

### Chemical Definitions

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transfbrmation such as by rearrangement, cyclization, elimination, etc.

“Z¹,” “Z²,” “Z³,” and “L⁴” are used herein as generic symbols to represent various specific substituents. These symbols can be any substituent, not limited to those disclosed herein, and when they are defined to be certain substituents in on instance, they can, in another instance, be defined as some other substituents.

As used herein, the term “acyl” refers to a group of formula —C(O)Z¹, where Z¹ is hydrogen, alkyl (e.g., C₁-C₁₀ haloalkyl (C₁-C₈ haloalkyl), alkenyl (C₂-C₈ alkenyl), haloalkenyl (e.g., C₂-C₈ haloalkenyl), alkynyl (e.g., C₂-C₈ alkynyl), alkoxy (C₁-C₈ alkoxy), haloalkoxyl (C₁-C₈ alkoxy), aryl, or heteroaryl, arylalkyl (C₇-C₁₀ arylalkyl), as defined below, where “C(O)” or “CO” is short-hand notation for C═O. A C(O) group is also referred to herein as a carbonyl. In some embodiments, the acyl group can be a C₁-C₆ acyl group (e.g., a formyl group, a C₁-C₅ alkylcarbonyl group, or a C₁-C₅ haloalkylcarbonyl group). In some embodiments, the acyl group can be a C₁-C₃ acyl group (e.g., a formyl group, a C₁-C₃ alkylcarbonyl group, or a C₁-C₃ haloalkylcarbonyl group).

As used herein, the term “alkyl” refers to straight-chained, branched, or cyclic, saturated hydrocarbon moieties. Unless otherwise specified, C₁-C₂₀ (e.g., C₁-C₁₂, C₁-C₁₀, C₁-C₈, C₁-C₆, C₁-C₄) alkyl groups are intended. Examples of alkyl groups include methyl, ethyl, propyl, isopropyl, 1-methyl-ethyl, butyl, isohutyl, t-butyl, 1-methyl-propyl, 2-methyl-propyl, 1,1-dimethyl-ethyl, pentyl, 1-methyl-butyl, 2-methyl-butyl, 3-methyl-butyl, 2,2-dimethyl-propyl, 1-ethyl-propyl, hexyl, 1,1-dimethyl-propyl, 1,2-dimethyl-propyl, methyl-pentyl, 2-methyl-pentyl, 3-methyl-pentyl, 4-methyl-pentyl, 1,1-dimethyl-butyl, 1,2-dimethyl-butyl, 1,3-dimethyl-butyl, 2,2-dimethyl-butyl, 2,3-dimethyl-butyl, butyl, 1-ethyl-butyl, 2-ethyl-butyl, 1,1,2-trimethyl-propyl, 1,2,2-trimethyl-propyl, 1-ethyl-1-methyl-propyl, 1-ethyl-2-methyl-propyl, pentyl, hexyl, heptyl, octyl, nonyl, decyl, dodecyl, tetradecyl, hexadecyl, eicosyl, tetracosyl, and the like. Alkyl substituents may be unsubstituted or substituted with one or more chemical moieties. Examples of suitable substituents include, for example, hydroxy, halogen, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylearbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, and haloalkylaminocarbonyl, provided that the substituents are sterically compatible and the rules of chemical bonding and strain energy are satisfied.

Throughout the specification “alkyl” is generally used to refer to both unsubstituted alkyl groups and substituted alkyl groups; however, substituted alkyl groups are also specifically referred to herein by identifying the specific substituent(s) on the alkyl group, For example, the term “halogenated alkyl” specifically refers to an alkyl_{—} group that is substituted with one or more halide, e.g., fluorine, chlorine, bromine, or iodine. The term specifically refers to an alkyl group that is substituted with one or more alkoxy gyoups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g,, an “alkylcycloalkyl.” a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkyicycloalkyl,” is not meant to imply that the general term does not also include the specific term.

As used herein, the term “haloalky ” refers to straight-chained or branched alkyl groups, wherein these groups the hydrogen atoms may partially or entirely be substituted with halogen atoms. Unless otherwise specified, C₁-C₂₀ (e.g., C₁-C₁₂, C₁-C₁₀, C₁-C₈, C₁-C₆, C₁-C₄) alkyl groups are intended. Examples include chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl, and 1,1,1-trifluoroprop-2-yl. Haloalkyl substituents may be unsubstituted or substituted with one or more chemical moieties. Examples of suitable substituents include, for example, hydroxy, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonylC₁-C₆ haloalkylcarbonyl, and haloalkylaminocarbonyl, provided that the substituents are sterically compatible and the rules of chemical bonding and strain energy are satisfied.

The term “cycloalkyl” as used herein is a non-aromatic carbon-based ring. Unless otherwise specified C₃-C₂₀ (e.g., C₃-C₁₂, C₃-C₁₀, C₃-C₈, C₃-C₆) cycloalkyl groups are intended. Examples of cycloalkyl groups include, but are not limited to, cyclopropyl, cyclohutyl, cyclopentyl, cyclohexyl, etc. The term “heterocycloalkyl” is a cycloalkyl group as defined above, and is included within the meaning of the term “cycloalkyl,” containing one or more heteroatoms, viz., N, O or S. The cycloalkyl or heterocycloalkyl substituents may be unsubstituted or substituted with one or more chemical moieties. Examples of suitable substituents include, fbr example, hydroxy, halogen, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, C₁-C₆ haloalkylcarbonyl and haloalkylaminocarbonyl, provided that the substituents are sterically compatible and the rules of chemical bonding and strain energy are satisfied.

As used herein, the term “alkenyl” refers to straight-chained, branched, or cyclic, unsaturated hydrocarbon moieties containing a double bond. Asymmetric structures such as (Z¹Z²)C═C(Z³Z⁴) are intended to include both the E and Z isomers. This can be presumed in structural formulae herein wherein an asymmetric alkene is present, or it can be explicitly indicated by the bond symbol C═C. Unless otherwise specified, C₂-C₂₀ (e.g., C₂-C₁₂, C₂-C₁₀, C₂-C₈, C₂-C₆, C₂-C₄) alkenyl groups are intended. Alkenyl groups may contain more than one unsaturated bond. Examples include ethenyl, 1-propenyl, 2-propenyl, 1-methylethenyl, 1-butenyl, 2-butertyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 12-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyt, 2-methyl-2-pentenyl, 3-methyl-2-pentenyt, 4-methyl-2-pentenyl, 1-methyl-3-pentenyl, 2-methyl.-3-pentenyl, 3-methyl-3-pentenyl, 4-methyl.-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-ditnethyl-1-butenyl, 2,3-dimethyl-2-butenyl, 2,3-dimethyl-3-butenyl, 3,3-dimethyll-butenyl, butenyl, 1-ethyl-2-butenyt, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl. The term “vinyl” refers to a group having the structure —CH═CH₂; 1-propenyt refers to a group with the structure —CH═CH—CH₃; and 2-propenyl refers to a group with the structure —CH₂—CH═CH₂. Alkenyl substituents may be unsubstituted or substituted with one or more chemical moieties. Examples of suitable substituents include, for example, hydroxy, halogen, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloaikenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, C₁-C₆haloalkylcarbonyl, and haloalkylaminocarbonyl, provided that the substituents are sterically compatible and the rules of chemical bonding and strain energy are satisfied.

The term “haloalkenyl,” as used herein, refers o an alkenyl group, as defined above, which is substituted by one or more halogen atoms.

The term “cycloalkenyl” as used herein is a non-aromatic carbon-based ring containing at least one double bond. Unless otherwise specified C₃-C₂₀ (e.g., C₃-C₁₂, C₃-C₁₀, C₃-C₈, C₃-C₆) cycloalkenyl groups are intended. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above, and is included within the meaning of the term “cycloalkenyl,” containing one or more heteroatoms, viz., N, O or S. The cycloalkenyl or heterocycloalkenyl substituents may be unsubstituted or substituted with one or more chemical moieties. Examples of suitable substituents include, for example, hydroxy, halogen, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloatkylthio, C₁-C₆ alkylaminocarbonyl, C₁-C₆ dialkylaminocarbonyl, C₁-C₆ haloalkoxycarbonyl, C₁-C₆ haloalkylcarbonyl, and haloalkylaminocarbonyl, provided that the substituents are sterically compatible and the rules of chemical bonding and strain energy are satisfied.

As used herein, the term “alkynyl” represents straight-chained or branched hydrocarbon moieties containing a triple bond. Unless otherwise specified, C₂-C₂₀ (e.g., C₂-C₁₂, C₂-C₁₀, C₂-C₈, C₂-C₆, C₂-C₄) alkynyl groups are intended. Alkynyl groups may contain more than one unsaturated bond. Examples include C₂-C₆-alkynyl, such as ethynyl, 1-propynyl, 2-propynyl (or propargyl), 1-butynyl, 2-butynyl, 3-butynyl, 1-methyl-2-propynyl, 1-pentynyl, 2-pentynyl, 3-pentynyl, 4-pentynyl, 3-methyl-1-butynyl, 1-methyl-2-butynyl, methyl-3-butynyl, 2-methyl-3-butynyl, 1,1-dimethyl-2-propynyl, 1-ethyl-2-propynyl, 1-hexynyl, 2-hexynyl, 3-hexynyl, 4-hexynyl, 5-hexynyl, 3-methyl-1-pentynyl, 4-methyl-1-pentynyl, 1-methyl-2-pentynyl, 4-methyl-2-pentynyl, 1-methyl-3-pentynyl, 2-methyl-3-pentynyl, 1-methyl-4-pentynyl, 2-methyl-4-pentynyl, 3-methyl-4-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl. Alkynyl substituents may be unsubstituted or substituted with one or more chemical moieties. Examples of suitable substituents include, for example, hydroxy, halogen, nitro, cyano, formyl, C₁-C₈ alkyl, C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆haloalkoxycarbonyl, C₁-C₆ haloalkylcarbonyl, and haloalkylaminocarbonyl, provided that the substituents are sterically compatible and the rules of chemical bonding and strain energy are satisfied.

As used herein, the term “alkoxy” refers to a group of the formula —OZ¹, where Z¹ is unsubstituted or substituted alkyl as defined above. In other words, as used herein an “alkoxy” group is an unsubstituted or substituted alkyl group bound through a single, terminal ether linkage. Unless otherwise specified, alkoxy groups wherein Z¹ is a C₁-C₂₀ (e.g., C₁-C₁₂, C₁-C₁₀, C₁-C₈, C₁-C₆, C₁-C₄) alkyl group are intended. Examples include methoxy, ethoxy, propoxy, 1-methyl-ethoxy, butoxy, 1-methyl-propoxy, 2-methyl-propoxy, 1,1-dimethyl-ethoxy, pentoxy, 1-methyl-butyloxy, 2-methyl-butoxy, 3-methyl-butoxy, 2,2-di-methyl-propoxy, 1-ethyl-propoxy, hexoxy, 1,1-dimethyl-propoxy, 2-dimethyl-propoxy, 1-methyl-pentoxy, 2-methyl-pentoxy, 3-methyl-pentoxy, 4-methyl-pentoxy, 1,1-dimethyl-butoxy, 1,2-dimethyl-butoxy, 1,3-dimethyl-butoxy, 2,2-dimethyl-butoxy, 2,3-dimethyl-butoxy, 3,3-dimethyl-butoxy, 1-ethyl-butoxy, 2-ethylbutoxy, 1,1,2-trimethyl-propoxy, 1,2,2-trimethyl-propoxy, 1-ethyl-1-methyl-propoxy, and 1-ethyl-2-methyl-propoxy.

As used herein, the term “haloalkoxy” refers to a group of the formula —OZ¹, where Z¹ is unsubstituted or substituted haloalkyl as defined above. Unless otherwise specified, haloalkoxy groups wherein Z¹ is a C₁-C₂₀ (e.g., C₁-C₁₂, C₁-C₁₀, C₁-C₈, C₁-C₆, C₁-C₄) alkyl group are intended. Examples include chloromethoxy, bromomethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 1-chloroethoxy, 1-bromoethoxy, 1-fluoroethoxy, 2-fluoroethoxy, 2,2-difluoroethoxy, 2,2,2-trifluoroethoxy, 2-chloro-2-fluoroethoxy, 2-chloro,2-difluoroethoxy, 2,2-dichloro-2-fluoroethoxy, 2,2,2-trichloroethoxy, pentafluoroethoxy, and 1,1,1-trifluoroprop-2-oxy.

As used herein, the term “aryl,” as well as derivative terms such as aryloxy, refers to groups that include a monovalent aromatic carbocyclic group of from 6 to 14 carbon atoms. Aryl groups can include a single ring or multiple condensed rings. In some embodiments, aryl groups include C₆-C₁₀ aryl groups. Examples of aryl groups include, but are not limited to, phenyl, biphenyl, naphthyl, tetrahydronaphtyl, phenylcyclopropyl, and indanyl. In some embodiments, the aryl group can be a phenyl, indanyl or naphthyl group. The term “heteroaryl”, as well as derivative terms such as “heteroaryloxy” refers to a 5- or 6-membered aromatic ring containing one or more heteroatoms, viz., N, O or S; these heteroaromatic rings may be fused to other aromatic systems. The aryl or heteroaryl substituents may be unsubstituted or substituted with one or more chemical moieties. Examples of suitable substituents include, for example, hydroxy, halogen, nitro, cyano, formyl, C₁-C₈ alkyl. C₁-C₈ haloalkyl, C₃-C₁₂ cycloalkyl, C₃-C₁₂ heterocycloalkyl, C₂-C₈ alkenyl, C₂-C₈ haloalkenyl, C₃-C₁₂ cycloalkenyl, C₃-C₁₂ heterocycloalkenyl, C₂-C₈ alkynyl, C₁-C₈ alkoxy, C₁-C₈ haloalkoxy, C₁-C₈ alkoxycarbonyl, hydroxycarbonyl, C₁-C₈ acyl, C₁-C₈ alkylcarbonyl, C₆-C₁₀ aryl, C₆-C₁₀ heteroaryl, amino, amido, C₁-C₈ carbamoyl, C₁-C₈ halocarbamoyl, phosphonyl, silyl, sulfinyl, C₁-C₆ alkylsulfinyl, C₁-C₆ haloalkylsulfinyl, sulfonyl, C₁-C₆ alkylsulfonyl, C₁-C₆ haloalkylsulfonyl, sulfonamide, thio, C₁-C₆ alkylthio, C₁-C₆ haloalkylthio, C₁-C₆ alkylaminocarbonyl, C₁-C₆ dialkylaminocarbonyl, C₁-C₆ haloalkoxycarbonyl, C₁-C₆ haloalkylcarbonyl, and haloalkylaminocarbonyl, provided that the substituents are sterically compatible and the rules of chemical bonding and strain energy are satisfied.

The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

As used herein, the term “arylalkyl” refers to an alkyl group substituted with an unsubstituted or substituted aryl group. C₇-C₁₀ arylalkyl refers to a group wherein the total number of carbon atoms in the group is 7 to 10, not including the carbon atoms present in any substituents of the aryl group.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

As used herein, the term “alkylcarbonyl” refers to an unsubstituted or substituted alkyl group bonded to a carbonyl group, wherein a carbonyl group is C(O). C₁-C₃ alkylcarbonyl and C₁-C₃ haloalkylcarbonyl refer to groups wherein a C₁-C₃ unsubstituted or substituted alkyl or haloalkyl group is bonded to a carbonyl group (the group contains a total of 2 to 4 carbon atoms).

As used herein, the term “alkoxycarbonyl” refers to a group of the formula

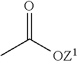

wherein can be a hydrogen, C₁-C₈ alkyl, C₁-C₈ haloalkyl, C₂-C₈ alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above.

The term “carboxylic acid” as used herein is represented by the formula —C(O)OH. A “carboxylate” or “carboxyl” group as used herein is represented by the formula —C(O)O⁻.

As used herein, the terms “amine” or “amino” refers to a group of the formula —NZ¹Z², where Z¹ and Z² can independently be a hydrogen, alkyl, haloalkyl, alkenyl, alkynyl, aryl, heteroaryl, cycloalkyl, cycloalkenyl, heterocycloalkyl, or heterocycloalkenyl group as described above. As used herein, the term “alkylamino” refers to an amino group substituted with one or two unsubstituted or substituted alkyl groups, which may be the same or different. As used herein, the term “haloalkylamino” refers to an alkylamino group wherein the alkyl carbon atoms are partially or entirely substituted with halogen atoms.

As used herein, “amido” refers to a group of the formula —C(O)NZ¹Z², where Z¹ and Z² can independently be a hydrogen, C₁-C₈alkyl, C₁-C₈ haloalkyl, C₂-C₈ alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above. As used herein, C₁-C₆ alkylaminocarbonyl refers to a group of the formula —C(O)NHZ¹ wherein Z¹ is C₁-C₆ unsubstituted or substituted alkyl. As used herein, C₁-C₆ dialkylaminocarbonyl refers to a group of the formula —C(O)N(Z¹)₂ wherein each Z¹ is independently C₁-C₆ unsubstituted or substituted alkyl.

As used herein, the term “carbamyl” (also referred to as carbamoyl and aminocarbonyl) refers to a group of the formula

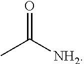

As used herein, the term “phosphonyl” refers to a group of the formula

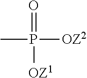

where Z¹ and Z² can independently be a hydrogen, C₁-C₈ alkyl, C₁-C₈ haloalkyl, C₂-C₈ alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above. As used herein “alkylphosphonyl” refers to a phosphonyl group substituted with one or two unsubstituted or substituted alkyl groups, which may be the same or different. As used herein, the term “haloalkylphosphonyl” refers to an alkylphosphonyl group wherein the alkyl carbon atoms are partially or entirely substituted with halogen atoms.

The term “silyl” as used herein is represented by the formula —SiZ¹Z²Z³, where Z¹, Z², and Z³ can be, independently, a hydrogen, C₁-C₈ alkyl, C₁-C₈ haloalkyl, C₂-C₈ alkenyl, C₂-C₈ alkynyl, C₆-C₁₀ aryl, C₆-C₁₀ heterowyl, C₃-C₁₂ cycloalkyl, C₃-C₁₂ cycloalkenyl, C₃-C₁₂ heterocycloalkyl, or C₃-C₁₂ heterocycloalkenyl group as described above. As used herein, C₁-C₆ trialkylsilyl refers to a group of the formula —Si(Z¹)₃ wherein each Z¹ is independently a C₁-C₆ unsubstituted or substituted alkyl group (the group contains a total of 3 to 18 carbon atoms).

As used herein, the term “sulfinyl” refers to a group of the formula

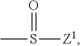

where Z¹ can be a hydrogen, C₁-C₈ alkyl, C₁-C₈ haloalkyl, C₂-C₈ alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above. The term “alkylsulfinyl” refers to a sultinyl group substituted with an unsubstituted or substituted alkyl group. As used herein, the term “haloalkylsulfinyl” refers to an alkylsulfinyl group wherein the alkyl carbon atoms are partially or entirely substituted with halogen atoms.

As used herein, the term “sulfonyl” refers to a group of the formula

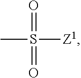

where Z¹ can be a hydrogen, C₁-C₈ alkyl, C₁-C₈ haloalkyl, alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above. The term “alkylsulfinyl” refers to a sulfinyl group substituted with an unsubstituted or substituted alkyl group. As used herein, the term “haloalkylsulfinyl” refers to an alkylsulfonyl group wherein the alkyl carbon atoms are partially or entirely substituted with halogen atoms.

The term “sulfonylamino” or “sulfonamide” as used herein is represented by the formula —S(O)₂NHZ¹, where Z¹ can be a hydrogen, C₁-C₈ alkyl, C₁-C₈ haloalkyl, C₂-C₈ alkenyl, C₂-C₈ alkvnyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above.

As used herein, the term “thio” refers to a group of the formula —SZ¹, where Z¹ can be a hydrogen, C₁-C₈ alkyl, C₁-C₈ haloalkyl, C₂-C₈ alkenyl, C₂-C₈ alkynyl, C₆-C₁₀ aryl, C₆-C₁₀ heteroaryl, C₃-C₁₂ cycloalkyl, C₃-C₁₂ cycloalkenyl, C₃-C₁₂ heterocycloalkyl, or C₃-C₁₂ heterocycloalkenyl group as described above.

The term “thiol” as used herein is represented by the formula —SH.

As used herein, the term “alkylthio” refers to a thio group substituted with an unsubstituted or substituted alkyl as defined above. Unless otherwise specified, alkylthio groups wherein the alkyl group is a C₁-C₂₀ (e.g., C₁-C₁₂, C₁-C₁₀, C₁-C₈, C₁-C₆, C₁-C₄) alkyl group are intended. Examples include methylthio, ethylthio, propylthio, 1-methylethylthio, butylthio, 1-methyl-propylthio, 2-methylpropylthio, 1,1-dimethylethylthio, pentylthio, 1-methylbutylthio, 2methylbutylthio, 3-methylbutylthio, 2,2-dio-methylpropylthio, 1-ethylpropylthio, hexylthio, 1,1-dimethyl propylthio, 1,2-dimethyl propylthio, 1-methylpentylthio, 2-methylpentylthio, 3-methyl-pentylthio, 4-methyl-pentylthio, 1,1-dimethyl butylthio, 1,2-dimethyl-butylthio, 1,3-dimethyl-butylthio, 2,2-dimethyl butylthio, 2,3-dimethyl butylthio, 3,3-dimethylbutylthio, 1-ethylbutylthio, 2-ethylbutylthio, 1,1,2-trimethyl propylthio, 1,2,2-trimethyl propylthio, 1-ethyl-1-methyl propylthio, and 1-ethyl-2-methylpropylthio.

As used herein, the term “haloalkylthio” refers to an alkylthio group as defined above wherein the carbon atoms are partially or entirely substituted with halogen atoms. Unless otherwise specified, haloalkylthio groups wherein the alkyl group is a C₁-C₂₀ (e.g., C₁-C₁₂, C₁-C₁₀, C₁-C₈, C₁-C₆, C₁-C₄) alkyl group are intended. Examples include chloromethyl thio, bromomethylthio, dichloromethylthio, trichloromethylthio, fluoromethylthio, difluoroniethylthio, trifluoromethylthio, chloroftuoromethylthio, dichlorofluoro-methylthio, chlorodifluorornethylthio, 1-chloroethylthio, 1-bromoethylthio, 1-fluoroethylthio, 2-fluoroethylthio, 2,2-difluoroethylthio, 2,2,2-trifluoroethylthio, 2-chloro-2-fluoroethylthio, 2-chloro-2-difluoroethylthio, 2,2-dichloro-2-fluoroethylthio, 2,2,2-trichloroethylthio, pentafluoroethylthio, and 1,1,1-trifluoroprop-2-ylthio.

As used herein, Me refers to a methyl group; OMe refers to a methoxy group; and i-Pr refers to an isopropyl group.

As used herein, the term “halogen” including derivative terms such as “halo” refers to fluorine, chlorine, bromine and iodine.

The term “hydroxyl” as used herein is represented by the formula —OH.

The term “nitro” as used herein is represented by the formula —NO₂.

“R¹,” “R²,” “R³,” “R⁴,” etc., where n is some integer, as used herein can, independently, possess one or more of the groups listed above, For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (a selected will determine if the first group is embedded or attached to the second group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A prodrug refers to a compound that is made more active in vivo. Certain compounds disclosed herein can also exist as prodrugs, as described in Hydrolysis in Drug and Prodrug Metabolism: Chemistiy, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they can be easier to administer than the compound, or parent drug. They can, for instance, be bioavallabie by oral administration whereas the parent drug is not. The prodrug can also have improved solubility in pharmaceutical compositions over the parent drug. A wide variety of prodrug derivatives are known in the art, such as those that rely on hydrolytic cleavage or oxidative activation of the prodrug,

Prodrugs of any of the disclosed compounds include, but are not limited to, carboxylate esters, carbonate esters, hemi-esters, phosphorus esters, nitro esters, sulfate esters, sulfoxides, amides, carbamates, azo compounds, phosphamides, glycosides, ethers, acetals, and ketals. Gligopeptide modifications and biodegradable polymer derivatives as described, for example, in Int. J. Pharm. 115, 61-67, 1995) are within the scope of the present disclosure. Methods for selecting and preparing suitable prodrugs are provided, for example, in the following: T. Higuchi and V. Stella, “Prodrugs as Novel Delivery Systems,” Vol. 14, ACS Symposium Series, 1975; H. Bundgaard, Design of Prodrugs, Elsevier, 1985; and Bioreversible Carriers in Drug Design, ed. Edward Roche, American Pharmaceutical Association and Pergamon Press, 1987.

Reference will now be made in detail to specific aspects of the disclosed materials, compounds, compositions, articles, and methods, examples of which are illustrated in the accompanying Examples and Figures.

### Compounds

Disclosed herein are arylnaphthalene lactone derivatives. Disclosed herein are compounds of Formula I:

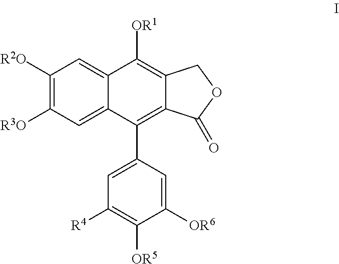

wherein

R¹ is hydrogen, halogen, nitro, cyano, formyl, substituted or unsubstituted alkyl, substituted or unsubstituted cycloalkyl, substituted or unsubstituted heterocycloalkyl, substituted or unsubstituted alkenyl, substituted or unstibstituted cycloalkenyl, substituted or unsubstituted heterocycloalkenyl, substituted or unsubstituted alkynyl, substituted or unsubstituted alkoxycarbonyl, hydroxycarbonyl, substituted or unsubstituted acyl, substituted or unsubstituted aryl, substituted or unsubstituted heteroaryl, substituted or unsubstituted amino, substituted or unsubstituted amido, substituted or unsubstituted carbamoyl, substituted or unsubstituted phosphonyl, substituted or unsubstituted substituted or unsubstituted sulfirtyl, substituted or unsubstituted sulfonyl, substituted or unsubstituted sulfonamide, or substituted or unsubstituted thio;

R² and R³ are independently hydrogen, halogen, substituted or unsubstituted substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R² and R³ taken together with the atoms to which they are attached form a substituted or unsttbstituted 5 to 7 membered heterocyclic moiety;

R⁴ is hydrogen, hydroxy, halogen, nitro, cyano, formyl, substituted or unsubstituted alkyl, substituted or unsubstituted cycloalkyl, substituted or unsubstituted heterocycloalkyl, substituted or unsubstituted alkenyl, substituted or unstibstituted cycloalkenyl, substituted or unsubstituted heterocycloalkenyl, substituted or unsubstitutcd alkynyl, substituted or unsubstituted alkoxy, substituted or unsubstituted alkoxycarbonyl, hydroxycarbonyl, substituted or unsubstituted acyl, substituted or unsubstituted aryl, substituted or unsubstituted heteroaryl, substituted or unsubstituted amino, substituted or unsubstituted amido, substituted or unsubstituted carbamoyl, substituted or unsubstituted phosphonyl, substituted or unsubstituted sityl, substituted or unsubstitutcd sulfinyl, substituted or unsubstituted sulfonyl, substituted or unsubstituted sulfonamide, or substituted or unsubstituted thio;

R⁵ and R⁶ are independently hydrogen, halogen, substituted or unstibstituted substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted suilinyl, substituted or unsubstituted sulfonyl, substituted or unsubstituted sulfonamide, substituted or unsubstituted thio, or R⁵ and R⁶ taken together with the atoms to which they are attached form a substituted or unsubstituted 5 to 7 membered heterocyclic moiety;

or a pharmaceutically acceptable salt or prodrug thereof.

In some examples of Formula I, can comprise a water solubilizing group. As used herein, a water solubilizing group is a functional group that can increase the solubilit:_{y}r of the compound in water. Examples of water solubilizing groups include, but are not limited to, phosphonyls, amino acids, succinate, poly(ethylene glycol), and the like, and combinations thereof.

In some examples of Formula R¹ is hydrogen, halogen, formyl, substituted or unsubstituted C₁-C₆ alkyl, substituted or unsubstituted C₄-C₁₀ cycloalkyl, substituted or unsubstituted C₄-C₁₀ heterocycloalkyl, substituted or unsubstituted alkoxycarbonyl, hydroxycarbonyl, or substituted or unsubstituted acyl.

In some examples of Formula I, R¹ is selected from:

### H, CH₃,

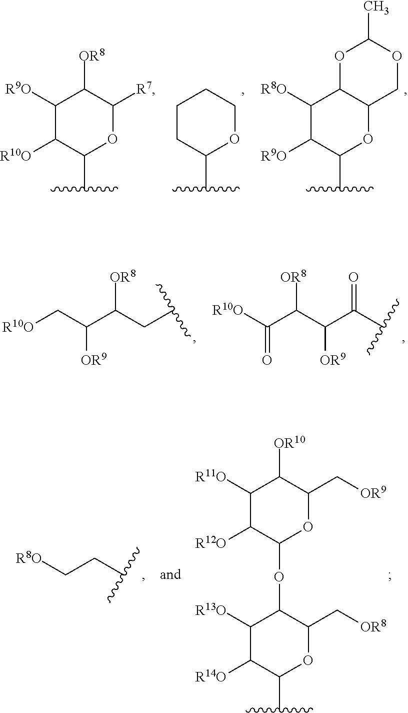

wherein, when present,

R⁷ is hydrogen, hydroxy, halogen, formyl, substituted or unsubstituted C₁-C₆ substituted or unsubstituted C₂-C₆ alkenyl, stibstituted or unsubstituted C₂-C₆ alkynyl, substituted or unsubstituted C₁-C₆ alkoxy, substituted or unsubstituted C₁-C₆ alkoxycarbonyl, hydroxyearbonyl, substituted or unsubstituted C₁-C₆ acyl, substituted or unsubstituted amino, substituted or unsubstituted amido, substituted or unsubstituted C₁-C₆ carbarnoyl, substituted or unsubstituted phosphonyl, substituted or unsubstituted silyl, substituted or unsubstituted sulfinyl, substituted or unsubstituted sulfonyl, substituted or unsubstituted sulfonamide, or substituted or unsubstituted thio; and

R⁸, R⁹, R¹⁰, R¹¹, R¹², R¹³, and R¹⁴ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆carbamoyl, substituted or unsubstituted phosphonyl, substituted or unsubstituted sulfinyl, substituted or unsubstituted sulfonyl, substituted or unsubstituted sulfonamide, or substituted or unsubstituted

In some examples of Formula I, R⁷ can comprise a water solubilizing group. In some examples of Formula I, R⁷ is hydrogen, substituted or unsubstituted C₁-C₆ alkyl, or substituted or unsubstituted C₁-C₆ acyl. In some examples of Formula I, R⁷ is hydrogen, CH₂C(O)CH₃, or CH₂OH.

In some examples of Formula I, one or more of R⁸-R ¹⁴ can comprise a water solubilizing group. In some examples of Formula I, R⁸, R⁹, R¹⁰, R¹¹, R¹², R¹³, and R¹⁴ are independently hydrogen, substituted or unsubstituted C₁-C₆ alkyl, substituted or unsubstituted C₁-C₆ acyl, or substituted or unsubstituted phosphonyl. In some examples of Formula I, R⁸, R⁹, R¹⁰, R¹¹, R¹², R¹³, and R¹⁴ are independently hydrogen, CH₃, C(O)CH₃, or PO₃H₂. In some examples of Formula I, R⁸, R⁹, R¹⁰, R¹¹, R¹², R¹³, and R¹⁴ are independently hydrogen, CH₃, or C(O)CH₃.

In some examples of Formula I, R¹ is

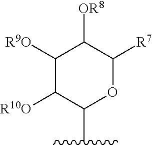

and one or more of R⁷-R¹⁰ can comprise a water sohibilizing group.

In some examples of Formula I, R¹ is

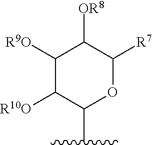

and R⁸, R⁹ and R¹⁰ are independently hydrogen, substituted or unsubstituted C₁-C₆ alkyl, substituted or unsubstituted C₁-C₆ aeyl, or substituted or unsubstituted phosphonyl.

In some examples of Formula I, R¹ is

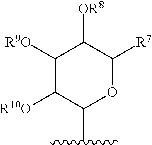

and R⁸, R⁹ and R¹⁰ are independently hydrogen, CH₃, C(O)CH₃, or PO₃H₂.

In some examples of Formula I, R¹ is

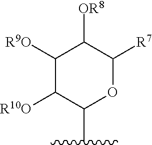

and R⁸, R⁹ and R¹⁰ are independently hydrogen, CH₃, or C(O)CH₃.

In some examples of Formula I, R¹ is

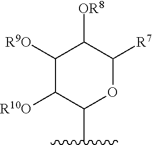

and R⁸ and R⁹ are H and R¹⁰ is CH₃.

In some examples of Formula I, R¹ is

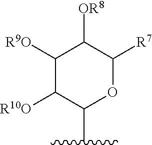

and R⁸ and R⁹ are C(O)CH₃ and R¹⁰ is H.

In some examples of Formula I, R¹ is

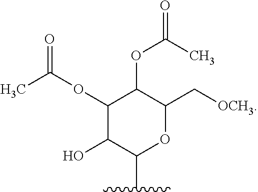 [SMILES: *C1OC(COC)C(OC(C)=O)C(OC(C)=O)C1O]

In some examples of Formula I, R² and/or R³ can comprise a water solubilizing group. In some examples of Formula I, R² and R³ are independently hydrogen, substituted or unsubstituted C₁-C₄ alkyl, or substituted or unsubstituted phosphonyl. In some examples of Formula I, R² and R³ are independently hydrogen, CH₃, or PO₃H₂.

In some examples of Formula I, R⁴ can comprise a water solubilizing group. In some examples of Formula I, R⁴ is hydrogen, hydroxy, substituted or unsubstituted C₁-C₆ alkyl, substituted or unsubstituted C₁-C₆ alkoxy, substituted or unsubstituted C₁-C₆ acyl, or substituted or unsubstituted phosphonyl. In some examples of Formula I, R⁴ is hydrogen.

In some examples of Formula I, R⁵ and/or R⁶ can comprise a water solubilizing group. In some examples of Formula I, R⁵ and R⁶ are independently hydrogen, substituted or unsubstituted C₁-C₄ alkyl, substituted or unsubstituted phosphonyl, or together with the atoms to which they are attached forma 5 membered heterocyclic group. In some examples of Formula I, R⁵ and R⁶ are independently hydrogen, CH₃, PO₃H₂, or together with the atoms to which they are attached form a 5 membered heterocyclic group. In some examples of Formula I, R⁵ and R⁶ together form a 5 membered heterocyclic group.

In some examples of Formula I, one or more of R¹-R¹⁴ can comprise a water solubilizing group. As used herein, a water solubi tizing group is a functional group that can increase the solubility of the compound in water. Examples of water solubilizing groups include, but are not limited to, phosphonyls, amino acids, succinate, poly(ethylene glycol), and the like, and combinations thereof.

In some examples of Formula I, the compounds are of Formula II:

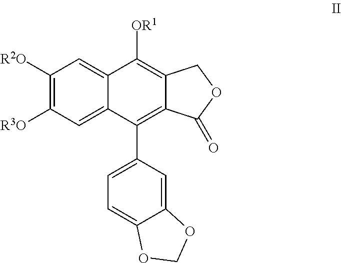

wherein

R¹ is hydrogen, halogen, nitro, cyano, formyl, substituted or unsubstituted alkyl, substituted or unsubstituted cycloalkyl, substituted or unsubstituted heterocycloalkyl, substituted or unsubstituted alkenyl, substituted or unsubstituted cycloalkenyl, substituted or unsubstituted heterocycloalkenyl, substituted or unsubstituted alkynyl, substituted or unsubstituted alkoxycarbonyl, hydroxycarbonyl, substituted or unsubstituted acyl, substituted or unsubstituted aryl, substituted or unsubstituted heteroaryl, substituted or unsubstituted amino, substituted or unsubstituted amido, substituted or unsubstituted carbamoyl, substituted or unsubstituted phosphonyl, substituted or unsubstituted substituted or unsubstituted sulfinyl, substituted or unsubstituted sulfonyl, substituted or unsubstituted sulfonamide, or substituted or unsubstituted thio;

R² and R³ are independently hydrogen, halogen, substituted or unsubstituted alkyl, substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted sulfinyl, substituted or unsubstituted suifonyl, substituted or unsubstituted sulfonamide, substituted or unsubstituted thio, or R² and R³ taken together with the atoms to which they are attached form a substituted or unsubstituted 5 to 7 membered heterocyclic moiety;

or a pharmaceutically acceptable salt or prodrug thereof.

In some examples of Formula II, R¹ can comprise a water solubilizing group. In some examples of Formula II, R¹ is hydrogen, halogen, formyl, substituted or unsubstituted C₁-C₆ alkyl, substituted or unsubstituted C₄-C₁₀ cycloalkyl, substituted or unsubstituted C₄-C₁₀ heterocycloalkyl, substituted or unsubstituted alkoxycarbonyl, hydroxycarbonyl, or substituted or unsubstituted acyl,

In some examples of Formula II, R¹ is selected from:

### H, CH₃,

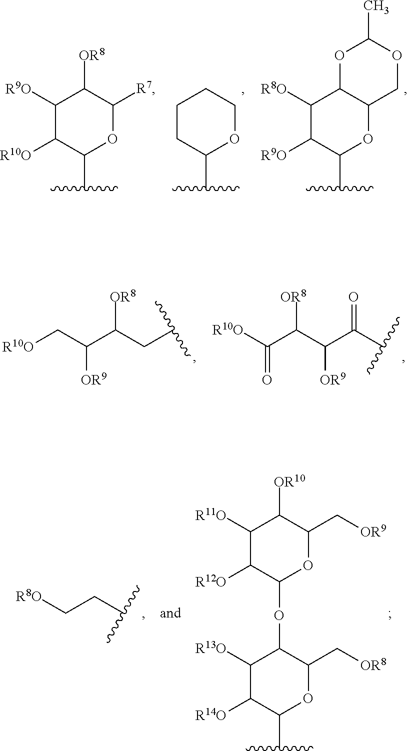

wherein, when present.

R⁷ is hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl hydroxycarbonyl, substituted or unsubstituted C₁-C₆ acyl, substituted or unsubstituted amino, substituted or unsubstituted amido, substituted or unsubstituted C₁-C₆ carbarnoyl, substituted or unsubstituted phosphonyl, substituted or unsubstituted silyl, substituted or unsubstituted sulfinyl, substituted or unsubstituted sulfonyl, substituted or unsubstituted sulfonamide, or substituted or unsubstituted thio; and

R⁸, R⁹, R¹⁰, R¹¹, R¹², R¹³, and R¹⁴ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted suifonyl, substituted or unsubstituted sulfonamide, or substituted or unsubstituted thio.

In some examples of Formula II, R⁷ can comprise a water solubilizing group. In some examples of Formula II, R⁷ is hydrogen, substituted or unsubstituted C₁-C₆ alkyl, or substituted or unsubstituted C₁-C₆ acyl. In some examples of Formula R⁷ is hydrogen, CH₂C(O)CH₃, or CH₂OH.

In some examples of Formula II, one or more of R⁸-R¹⁴ can comprise a water solubilizing group. In some examples of Formula II, R⁸, R⁹, R¹⁰, R¹¹, R¹², R¹³, and R¹⁴ are independently hydrogen, substituted or unsubstituted C₁-C₆ alkyl, substituted or unsubstituted C₁-C₆ acyl, or substituted or unsubstituted phosphonyl. In some examples of Formula II, R⁸, R⁹, R¹⁰, R¹¹, R¹², R¹³, and R¹⁴ are independently hydrogen, CH₃, C(O)CH₃, or PO₃H₂. In some examples of Formula II, R⁸, R⁹, R¹⁰, R¹¹, R¹², R¹³, and R¹⁴ are independently hydrogen, CH₃, or C(O)CH₃.

In some examples of Formula II, R¹ is

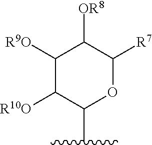

and one or more of R⁷-R¹⁰ is a water soltibilizing group.

In some examples of Formula II, R¹ is

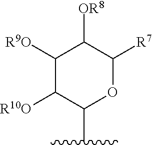

and R⁸, R⁹ and R¹⁰ are independently hydrogen, substituted or unsubstituted C₁-C₆ alkyl, substituted or unsubstituted aeyl, or substituted or unsubstituted phosphonyl.

In some examples of Formula II, R¹ is

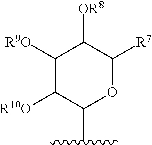

and R⁸, R⁹ and R¹⁰ are independently hydrogen, CH₃, C(O)CH₃, or PO₃H₂.

In some examples of Formula II, R¹ is

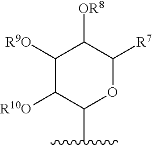

and R⁸, R⁹ and R¹⁰ are independently hydrogen, CH₃, or C(O)CH₃.

In some examples of Formula II, R¹ is

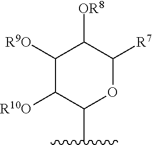

and R⁸ and R⁹ are H and R¹⁰ is CH₃.

In es some exampl of Formula II, R¹ is

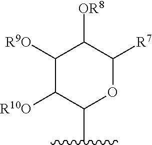

and R⁸ and R⁹ are C(O)CH₃ and R¹⁰ is H.

In some examples of Formula II, R¹ is

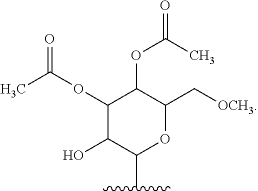 [SMILES: *C1OC(COC)C(OC(C)=O)C(OC(C)=O)C1O]

In some examples of Formula II, R² and/or R³ can comprise a water solubilizing group. In some examples of Formula II, R² and R3 are independently hydrogen, substituted or unsubstituted C₁-C₄ alkyl, or substituted or unsubstituted phosphonyl. In some examples of Formula II, R² and R³ are independently hydrogen, CH₃, or PO₃H₂. In some examples of Formula II, R² is CH₃. In some examples of Formula II, R³ is CH₃.

In some examples of Formula II, the compounds are of Formula II-A:

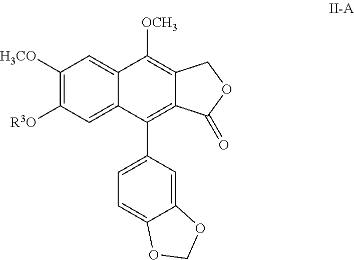

wherein

R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula II-A, R³ can comprise a water solubilizing group. In some examples of Formula II-A, R³ is hydrogen, substituted or unsubstituted C₁-C₄ alkyl, or substituted or unsubstituted phosphonyl. In some examples of Formula II-A, R³ is hydrogen, CH₃, or PO₃H₂.

In some examples of Formula II-A, the compounds are of Formula II-A-1:

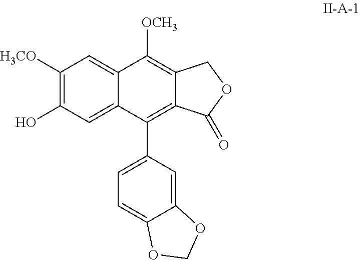 [SMILES: COc1cc2c(OC)c3c(c(-c4ccc5c(c4)OCO5)c2cc1O)C(=O)OC3]

or a pharmaceutically acceptable salt or prodrug thereof.

In some examples of Formula II, the compounds are of Formula II-B:

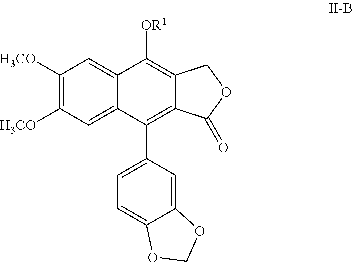

wherein

R¹ is hydrogen, halogen, formyl, substituted or unsubstituted C₁-C₆ alkyl, substituted or unsubstituted C₄-C₁₀ cycloalkyl, substituted or unsubstituted heterocycloalkyl, substituted or unsubstituted alkoxycarbonyl, hydroxycarbonyl, substituted or unsubstituted acyl;

or a pharmaceutically acceptable salt or prodrug thereof.

In some examples of Formula II-B, R¹ can comprise a water solubilizing group.

In some examples of Formula II-B, R¹ is selected from:

### H,

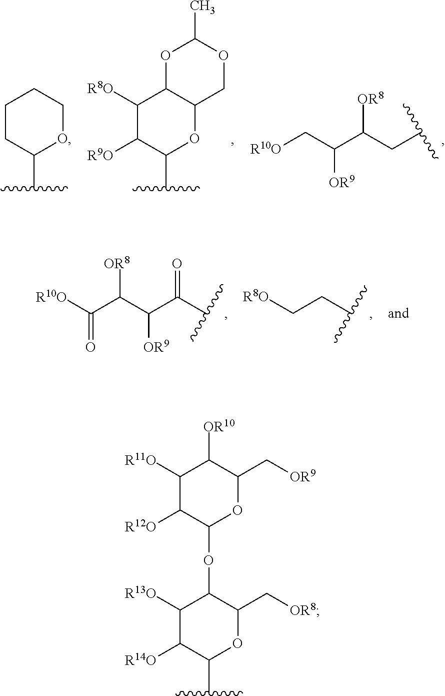

wherein, when present,

R⁸, R⁹, R¹⁰, R¹¹, R¹², R¹³, and R¹⁴ are independently hydrogen, halogen, substituted or unsubstituted C₁-C₆ alkyl, substituted or unsubstituted C₁-C₆ alkoxycarbonyl, hydroxyearbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio.

In some examples of Formula II-B, one or more of R⁸-R¹⁴ can comprise a water solubilizing group in some examples of Formula II-B, R⁸, R⁹, R¹⁰, R¹¹, R¹², R¹³ and R¹⁴ are independently hydrogen, substituted or unsubstituted C₁-C₆ alkyl, substituted or unsubstituted C₁-C₆ acyl, or substituted or unsubstituted phosphonyl. In some examples of Formula I, R⁸, R⁹, R¹⁰, R¹¹, R¹², R¹³, and R¹⁴ are independently hydrogen, CH₃, C(O)CH₃, or PO₃H₂. In some examples of Formula I, R⁸, R⁹, R¹⁰, R¹¹, R¹², R¹³, and R¹⁴ are independently hydrogen, CH₃, or C(O)CH₃.

In some examples of Formula II-B, R¹ is selected from:

### H,

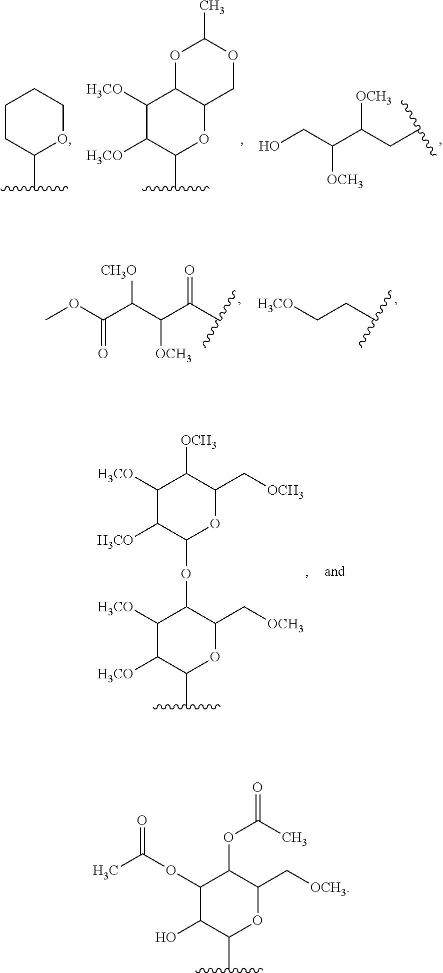

In some examples of Formula II-B, the compound is of Formula II-B-1:

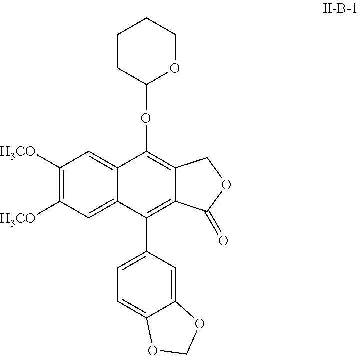 [SMILES: COc1cc2c(OC3CCCCO3)c3c(c(-c4ccc5c(c4)OCO5)c2cc1OC)C(=O)OC3]

or a pharmaceutically acceptable salt or prodrug thereof.

In some examples of Formula II-B, the compound is of Formula II-B-2:

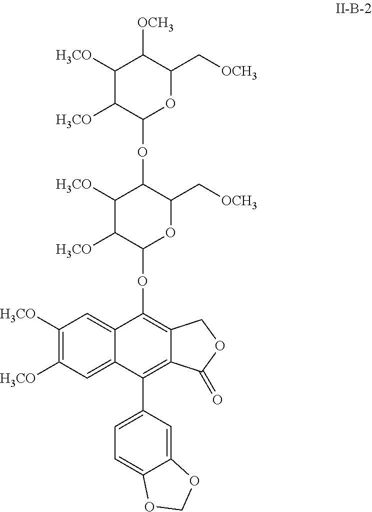 [SMILES: COCC1OC(OC2C(COC)OC(Oc3c4c(c(-c5ccc6c(c5)OCO6)c5cc(OC)c(OC)cc35)C(=O)OC4)C(OC)C2OC)C(OC)C(OC)C1OC]

or a pharmaceutically acceptable salt or prodrug thereof.

In some examples of Formula II-B, the compound is of Formula II-B-3:

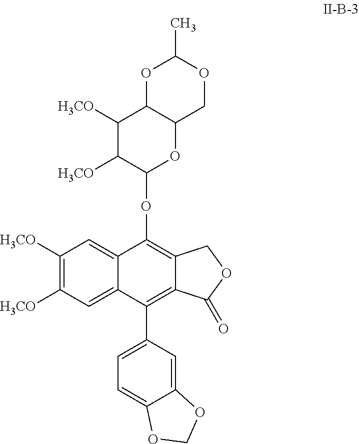 [SMILES: COc1cc2c(OC3OC4COC(C)OC4C(OC)C3OC)c3c(c(-c4ccc5c(c4)OCO5)c2cc1OC)C(=O)OC3]

or a pharmaceutically acceptable salt or prodrug thereof.

In some examples of Formula II-B, the compound is of Formula II-B-4:

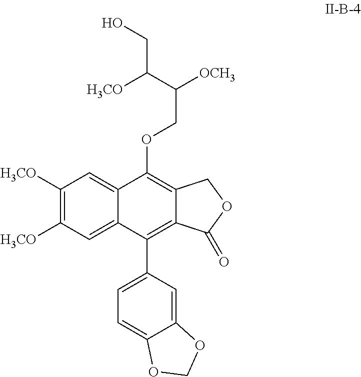 [SMILES: COc1cc2c(OCC(OC)C(CO)OC)c3c(c(-c4ccc5c(c4)OCO5)c2cc1OC)C(=O)OC3]

or a pharmaceutically acceptable salt or prodrug thereof.

In some examples of Formula II-B, the compound is of Formula II-B-5:

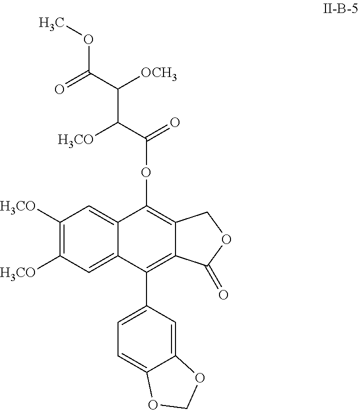 [SMILES: COC(=O)C(OC)C(OC)C(=O)Oc1c2c(c(-c3ccc4c(c3)OCO4)c3cc(OC)c(OC)cc13)C(=O)OC2]

or a pharmaceutically acceptable salt or prodrug thereof.

In some examples of Formula II-B, the compounds of Formula II-B-6:

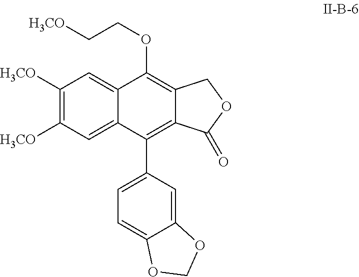 [SMILES: COCCOc1c2c(c(-c3ccc4c(c3)OCO4)c3cc(OC)c(OC)cc13)C(=O)OC2]

or a pharmaceutically acceptable salt or prodrug thereof.

In some examples of Formula II-B, the compound s of Formula II-B-7:

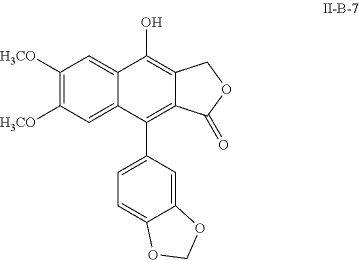 [SMILES: COc1cc2c(O)c3c(c(-c4ccc5c(c4)OCO5)c2cc1OC)C(=O)OC3]

or a pharmaceutically acceptable salt or prodrug thereof.

In some examples of Formula II-B, the compound s of Formula II-B-8:

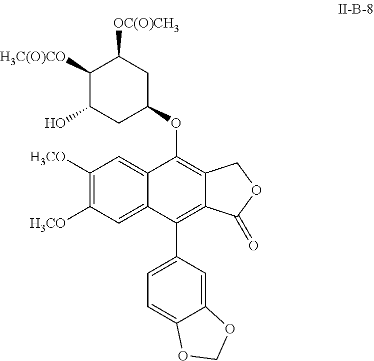

or a pharmaceutically acceptable salt or prodrug thereof.

In some examples of Formula I, the compounds are of Formula III:

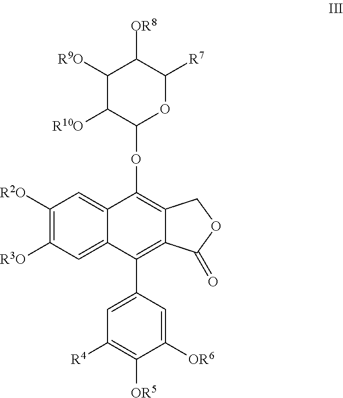

wherein

R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio; or R² and R³ taken together with the atoms to which they are attached form a substituted or unsubstituted 5 to 7 membered heterocyclic moiety;

R⁴ and R⁷ are independently hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted silyl, substituted or unsubstituted sulfinyl, substituted or unsubstituted sulfonyl, substituted or unsubstituted sulfonamide, or substituted or unsubstituted thio;

R⁵ and R⁶ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R⁵ and R⁶ taken together with the atoms to which they are attached form a substituted or unsubstituted 5 to 7 membered heterocyclic moiety;

R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula III, R² and/or R³ can comprise a water solubilizing group. In some examples of Formula III, R² and R³ are independently hydrogen, substituted or unsubstituted C₁-C₄ alkyl, or substituted or unsubstituted phosphonyl. In some examples of Formula III, R² and R³ are independently hydrogen, CH₃, or PO₃H₂.

In some examples of Formula III, R⁴ n m can comprise a water solubilizing group. In some examples of Formula III, R⁴ is hydrogen, hydroxy, substituted or unsubstituted C₁-C₆ alkyl, substituted or unsubstituted C₁-C₆ alkoxy, substituted or unstibstituted C₁-C₆ acyl, or substituted or unsubstituted phosphonyl. In some examples of Formula III, R⁴ is hydrogen.

In some examples of Formula III, R⁵ and/or R⁶ can comprise a water solubilizing group. In some examples of Formula III, R⁵ and R⁶ are independently hydrogen, substituted or unsubstituted C₁-C₄ alkyl, substituted or unsubstituted phosphonyl, or together with the atoms to which they are attached form a 5 membered heterocyclic group. In some examples of Formula III, R⁵ and R⁶ are independently hydrogen, CH₃, PO₃H₂, or together with the atoms to which they are attached forma 5 membered heterocyclic group. In some examples of Formula III, R⁵ and R⁶ together form a 5 membered heterocyclic group.

In some examples of Formula III, R⁷ can comprise a water solubilizing group. In some examples of Formula III, R⁷ is hydrogen, substituted or unsubstituted C₁-C₆ alkyl, or substituted or unsubstituted C₁-C₆ acyl. In some examples of Formula III, R⁷ is hydrogen, CH₂C(O)CH₃, or CH₂OH.

In some examples of Formula III, one or more of R⁸-R⁹ can comprise a water solubilizing group. In some examples of Formula III, R⁸, R⁹ and R¹⁰ are independently hydrogen, substituted or unsubstituted C₁-C₆ alkyl, substituted or unsubstituted C₁-C₆ acyl, or substituted or unsubstituted phosphonyl. In some examples of Formula III, R⁸, R⁹ and R¹⁰ are independently hydrogen, CH₃, C(O)CH₃, or PO₃H₂. In some examples of Formula III, R⁸, R⁹ and R¹⁰ are independently hydrogen, CH₃, or C(O)CH₃. In some examples of Formula III, R⁸ and R⁹ are H and R¹⁰ is CH₃. In some examples of Formula III, R⁸ and R⁹ are C(O)CH₃ and R¹⁰ is H.

In some examples of Formula III, the compounds are of Formula IV:

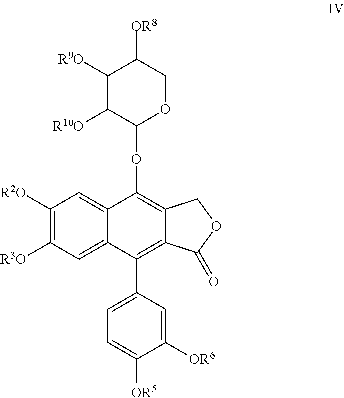

wherein

R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

R⁵ and R⁶ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R⁵ and R⁶ taken together with the atoms to which they are attached form a substituted or unsubstituted 5 to 7 membered heterocyclic moiety;

R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted atnido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula IV, R² and/or R³ can comprise a water solubilizing group. In some examples of Formula IV, R² and R³ are independently hydrogen, substituted or unsubstituted C₁-C₄ alkyl, or substituted or unsubstituted phosphonyl. In some examples of Formula IV, R² and R³ are independently hydrogen, CH₃, or PO₃H₂.

In some examples of Formula IV, R⁵ and/or R⁶ can comprise a water solubilizing group. In some examples of Formula IV, R⁵ and R⁶ are independently hydrogen, substituted or unsubstituted C₁-C₄ alkyl, substituted or unsubstituted phosphonyl, or together with the atoms to which they are attached form a 5 membered heterocyclic group. In some examples of Formula IV, R⁵ and R⁶ are independently hydrogen, CH₃, PO₃H₂, or together with the atoms to which they are attached forma 5 membered heterocyclic group.

In some examples of Formula IV, R² is CH₃. In some examples of Formula IV, R³ is CH₃. In some examples of Formula IV, R⁶ is CH₃. In some examples of Formula IV, R² and R³ are CH₃. In some examples of Formula IV, R² and R⁶ are CH₃. In some examples of Formula IV, R³ and R⁶ are CH₃.

In some examples of Formula IV, one or more of R⁸-R¹⁰ can comprise a water solubilizing group. In some examples of Formula IV, R⁸, R⁹ R¹⁰ are independently hydrogen, substituted or unsubstituted C₁-C₆ alkyl, substituted or unsubstituted C₁-C₆ acyl, or substituted or unsubstituted phosphonyl. In some examples of Formula IV, R⁸, R⁹ and R¹⁰ are independently hydrogen, CH₃, C(O)CH₃, or PO₃H₂. In some examples of Formula IV, R⁸, R⁹ and R¹⁰ are independently hydrogen, CH₃, or C(O)CH₃.

In some examples of Formula IV, the compounds are of Formula IV-A:

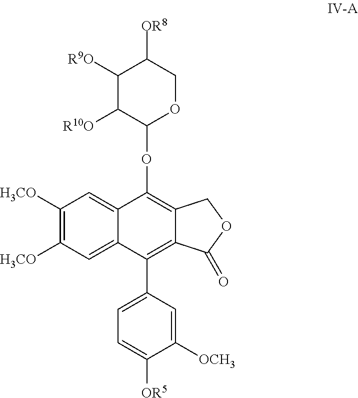

wherein

R⁵ is hydrogen, halogen, substituted or unsubstituted alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsitbstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula IV-A, R⁵ is a water solubilizing group. In some examples of Formula IV-A, R⁵ is hydrogen, substituted or unsubstituted C₁-C₄ alkyl, or substituted or unsubstituted phosphonyl. In some examples of Formula IV-A, R⁵ is hydrogen, CH₃, or PO₃H₂.

In some examples of Formula IV-A, one or more of R⁸-R¹⁰ can comprise a water solubilizing group. In some examples of Formula IV-A, R⁸, R⁹ and R¹⁰ are independently hydrogen, substituted or unsubstituted C₁-C₆ alkyl, substituted or unsubstituted C₁-C₆ acyl, or substituted or unstibstituted phosphonyl. In some examples of Formula IV-A, R⁸, R⁹ and R¹⁰ are independently hydrogen, CH₃, C(O)CH₃, or PO₃H₂. In some examples of Formula IV-A, R⁸, R⁹ and R¹⁰ are independently hydrogen, CH₃, or C(O)CH₃.

In some examples of Formula IV-A, compounds are of Formula IV-B:

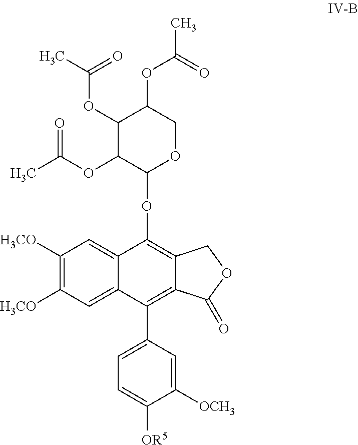

wherein

R⁵ is hydrogen, halogen, substituted or unsubstituted C₁-C₄ alkyl substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unstibstituted thio;

or a pharmaceutically acceptable salt or prodrug thereof.

In some examples of Formula IV-B, R⁵ is a water solubilizing group. In some examples of Formula IV-B, R⁵ is hydrogen, substituted or unsubstituted C₁-C₄ alkyl, or substituted or unsubstituted phosphonyl. In some examples of Formula IV-B, R⁵ is hydrogen, CH₃, or PO₃H₂.

In some examples of Formula IV-B, the compound is of Formula IV-B-1:

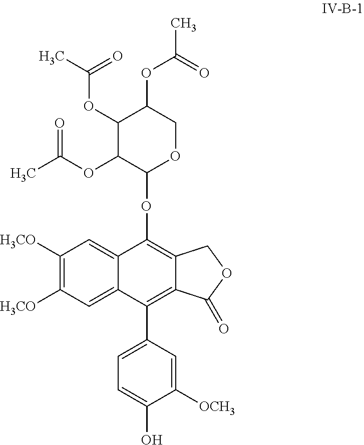 [SMILES: COc1cc(-c2c3c(c(OC4OCC(OC(C)=O)C(OC(C)=O)C4OC(C)=O)c4cc(OC)c(OC)cc24)COC3=O)ccc1O]

or a pharmaceutically acceptable salt or prodrug thereof.

In some examples of Formula IV-B, the compound is of Formula IV-B-2:

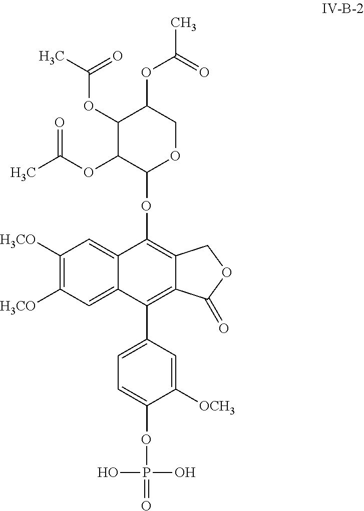 [SMILES: COc1cc2c(OC3OCC(OC(C)=O)C(OC(C)=O)C3OC(C)=O)c3c(c(-c4ccc(OP(=O)(O)O)c(OC)c4)c2cc1OC)C(=O)OC3]

or a pharmaceutically acceptable salt or prodrug thereof.

In some examples of Formula IV-A, compounds are of Formula IV-C:

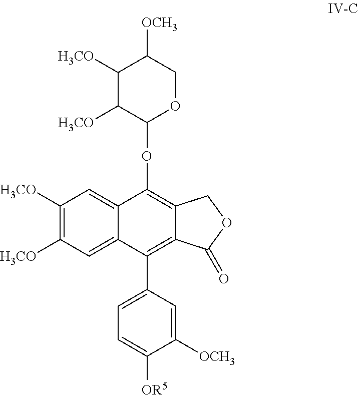

wherein

R⁵ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula IV-C, R⁵ is a water solubilizing group. In some examples of Formula IV-C, R⁵ is hydrogen, substituted or unsubstituted C₁-C₄ alkyl, or substituted or unsubstituted phosphonyl. In some examples of Formula IV-C, R⁵ is hydrogen, CH₃, or PO₃H₂.

In some examples of Formula IV-C, compounds are of Formula IV-C-1:

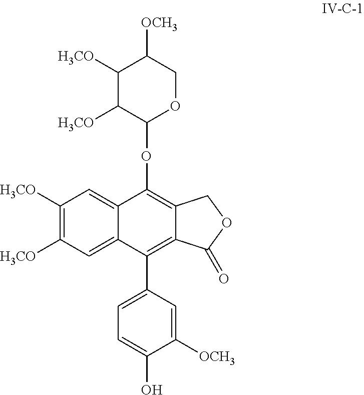 [SMILES: COc1cc(-c2c3c(c(OC4OCC(OC)C(OC)C4OC)c4cc(OC)c(OC)cc24)COC3=O)ccc1O]

or a pharmaceutically acceptable salt or prodrug thereof.

In some examples of Formula IV-C, compounds are of Formula IV-C-2:

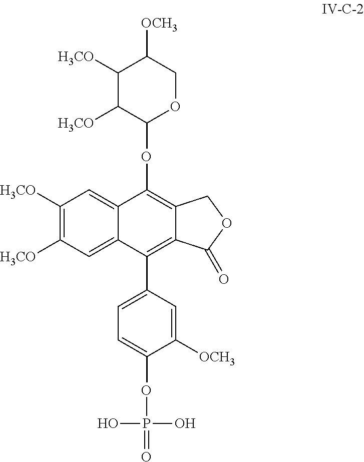 [SMILES: COc1cc2c(OC3OCC(OC)C(OC)C3OC)c3c(c(-c4ccc(OP(=O)(O)O)c(OC)c4)c2cc1OC)C(=O)OC3]

or a pharmaceutically acceptable salt or prodrug thereof.

In some examples of Formula IV, compounds are of Formula V:

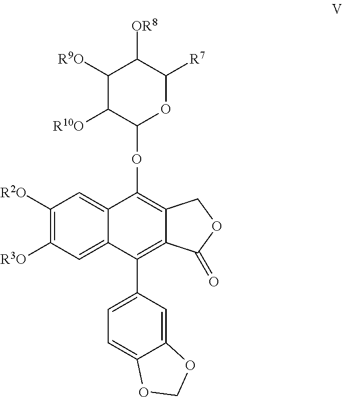

wherein

R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, is substituted or unsubstituted sulfonamide, or substituted or unsubstituted thio; or R² and R³ taken together with the atoms to which they are attached form a substituted or unsubstituted 5 to 7 membered heterocyclic moiety;

R⁷ is hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted silyl, substituted or unsubstituted sulfirtyl, substituted or unsubstituted sulfonyl, substituted or unsubstituted sulfonamide, or substituted or unsubstituted thio;

R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula V, R² and/or R³ can comprise a water solubilizing group. In some examples of Formula V, R² and R³ are independently hydrogen, substituted or unsubstituted C₁-C₄ alkyl, or substituted or unsubstituted phosphonyl. In some examples of Formula V, R² and R³ are independently hydrogen, CH₃, or PO₃H₂.

In some examples of Formula V, R⁵ can comprise a water solubilizing group. In some examples of Formula V, R⁷ is hydrogen, substituted or unsubstituted C₁-C₆ alkyl, or substituted or unsubstituted C₁-C₆ acyl. In some examples of Formula V, R⁷ is hydrogen, CH₂C(O)CH₃, or CH₂OH.

In some examples of Formula V, one or more of R⁸-R¹⁰ can comprise a water solubilizing group. In some examples of Formula V, R⁸, R⁹ and R¹⁰ are independently hydrogen, substituted or unsubstituted C₁-C₆ alkyl, substituted or unsubstituted C₁-C₆ acyl, or substituted or unsubstituted phosphonyl. In some examples of Formula V, R⁸, R⁹, and R¹⁰ are independently hydrogen, CH₃, C(O)CH₃, or PO₃H₂. In some examples of Formula V, R⁸, R⁹, and R¹⁰ are independently hydrogen, CH₃, or C(O)CH₃.

In some examples of Formula V, compounds are of Formula V-A:

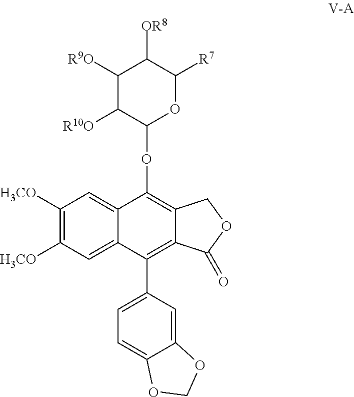

wherein

R⁷ is hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unstibstituted C₁-C₆ carbamoyl, substituted or unsubstituted phosphonyl, substituted or unsubstituted silyl, substituted or unsubstituted sulfinyl, substituted or unsubstituted suifonyl, substituted or unsubstituted sulfonamide, or substituted or unsubstituted thio;

R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unstibstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula V-A, R⁷ can comprise a water solubilizing group. In some examples of Formula V-A, R⁷ is hydrogen, substituted or unsubstituted C₁-C₆ alkyl, or substituted or unsubstituted C₁-C₆ acyl. In some examples of Formula V-A, R⁷ is hydrogen, CH₂C(O)CH₃, or CH₂OH.

In some examples of Formula V-A, one or more of R⁸-R¹⁰ can comprise a water solubilizing group. In some examples of Formula V-A, R⁸, R⁹ and R¹⁰ are independently hydrogen, substituted or unsubstituted C₁-C₆ alkyl, substituted or unsubstituted C₁-C₆ acyl, or substituted or unsubstituted phosphonyl. In some examples of Formula V-A, R⁸, R⁹ and R¹⁰ are independently hydrogen, CH₃, C(O)CH₃, or PO₃H₂. In some examples of Formula V-A, R⁸, R⁹ and R¹⁰ are independently hydrogen, CH₃, or C(O)CH₃.

In some examples of V-A, one or more of R⁷-R¹⁰ can comprise a water solubilizing group.

In some examples of Formula V-A, the compound is of Formula V-A-1:

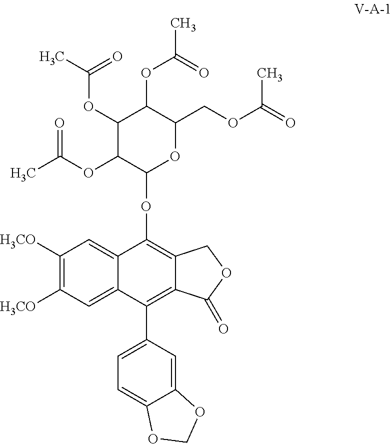 [SMILES: COc1cc2c(OC3OC(COC(C)=O)C(OC(C)=O)C(OC(C)=O)C3OC(C)=O)c3c(c(-c4ccc5c(c4)OCO5)c2cc1OC)C(=O)OC3]

or a pharmaceutically acceptable salt or prodrug thereof.

In some examples of Formula V-A, the compound is of Formula V-A-2:

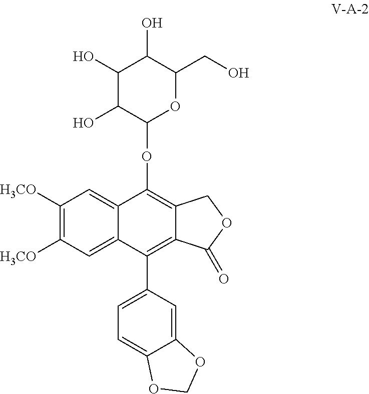 [SMILES: COc1cc2c(OC3OC(CO)C(O)C(O)C3O)c3c(c(-c4ccc5c(c4)OCO5)c2cc1OC)C(=O)OC3]

or a pharmaceutically acceptable salt or prodrug thereof.

In some examples of Formula V, the compounds are of Formula VI:

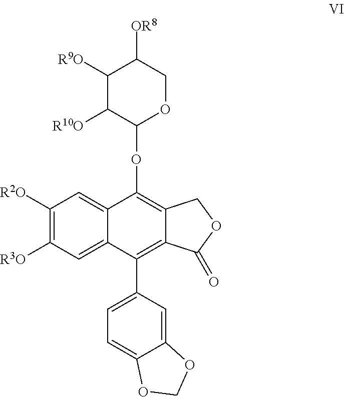

wherein

R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio; or R² and R³ taken together with the atoms to which they are attached form a substituted or unsubstituted 5 to 7 membered heterocyclic moiety;

R⁸, R⁹, and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula VI, R² and/or R³ can comprise a water solubilizing group. In some examples of Formula VI, R² and R³ are independently hydrogen, substituted or unsubstituted C₁-C₄ alkyl, or substituted or unsubstituted phosphonyl. In some examples of Formula VI, R² and R³ are independently hydrogen, CH₃, or PO₃H₂.

In some examples of Formula VI, one or more of R⁸-R¹⁰ can comprise a water solubilizing group. In some examples of Formula VI, R⁸, R⁹ and R¹⁰ are independently hydrogen, substituted or unsubstituted C₁-C₆ alkyl, substituted or unsubstituted C₁-C₆ acyl, or substituted or unsubstituted phosphonyl. In some examples of Formula VI, R⁸, R⁹ and R¹⁰ are independently hydrogen, CH₃, C(O)CH₃, or PO₃H₂.

In some examples of Formula VI, the compounds are of Formula VI-A:

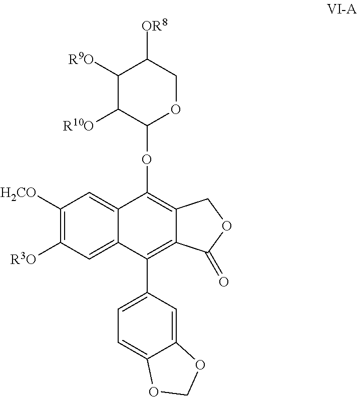

wherein

R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbarnoyl, substituted or unsubstituted phosphonyl, substituted or unsubstituted sulfinyl, substituted or unsubstituted sulfonyl, substituted or unsubstituted sulfonamide, or substituted or unsubstituted thio;

R⁸, R⁹ and R¹⁰ are independently hydrogen, halogen, substituted or unstibstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula VI-A, R³ can comprise a water solubilizing group. In some examples of Formula VI-A, R³ is hydrogen, substituted or unsubstituted C₁-C₄ alkyl, or substituted or unsubstituted phosphonyl. In some examples of Formula VI-A, R³ is hydrogen, CH₃, or PO₃H₂.

In some examples of Formula VI-A, one or more of R⁸-R¹⁰ can comprise a water solubilizing group. In some examples of Formula VI-A, R⁸, R⁹ and R¹⁰ are independently hydrogen, substituted or unsubstituted C₁-C₆ alkyl, substituted or unsubstituted C₁-C₆ acyl, or substituted or unsubstituted phosphonyl. In some examples of Formula VI-A, R⁸, R⁹ and R¹⁰ are independently hydrogen, CH₃, C(O)CH₃, or PO₃H₂. In some examples of Formula VI-A, R⁸, R⁹ and R¹⁰ are independently hydrogen, CH₃, or C(O)CH₃.

In some examples of Formula VI-A, the compounds are of Formula VI-B:

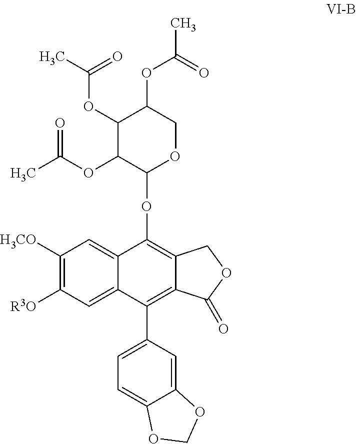

wherein

R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula VI-B, R³ is a water solubilizing group. In some examples of Formula VI-B, R³ is hydrogen, substituted or unsubstituted C₁-C₄ alkyl, or substituted or unsubstituted phosphonyl. In some examples of Formula VI-B, R³ is hydrogen, CH₃, or PO₃H₂.

In some examples of Formula VI-B, the compounds are of Formula VI-B-1:

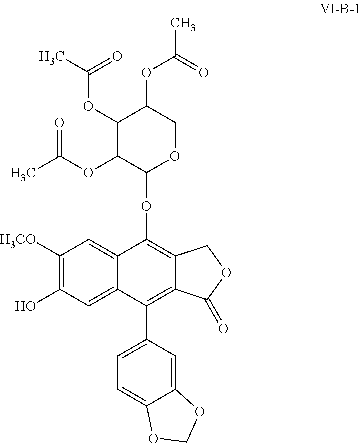 [SMILES: COc1cc2c(OC3OCC(OC(C)=O)C(OC(C)=O)C3OC(C)=O)c3c(c(-c4ccc5c(c4)OCO5)c2cc1O)C(=O)OC3]

or a pharmaceutically acceptable salt or prodrug thereof.

In some examples of Formula VI-B, the compounds are of Formula a VI-B-2:

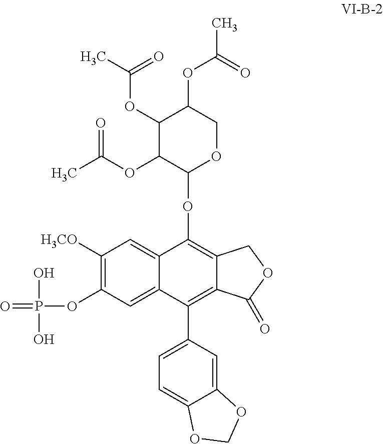 [SMILES: COc1cc2c(OC3OCC(OC(C)=O)C(OC(C)=O)C3OC(C)=O)c3c(c(-c4ccc5c(c4)OCO5)c2cc1OP(=O)(O)O)C(=O)OC3]

or a pharmaceutically acceptable salt or prodrug thereof.

In some examples of Formula VI-A, the compounds are of Formula VI-C:

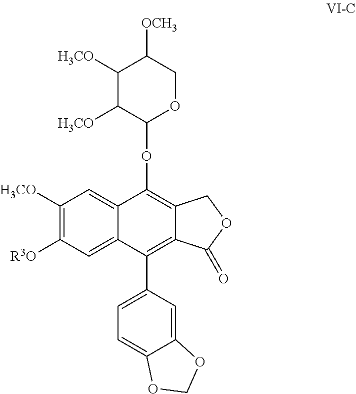

wherein

R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula VI-C, R³ is a water solubilizing group. In some examples of Formula VI-C, R³ is hydrogen, substituted or unsubstituted C₁-C₄ alkyl, or substituted or unsubstituted phosphonyl. In some examples of Formula VI-C, R³ is hydrogen, CH₃, or PO₃H₂.

In some examples of Formula VI-C, the compound is of Formula VI-C-1:

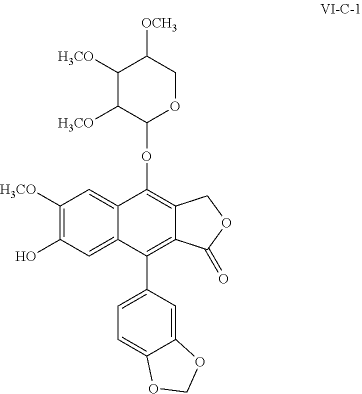 [SMILES: COc1cc2c(OC3OCC(OC)C(OC)C3OC)c3c(c(-c4ccc5c(c4)OCO5)c2cc1O)C(=O)OC3]

or a pharmaceutically acceptable salt or prodrug thereof.

In some examples of Formula VI-C, the compound is of Formula VI-C-2:

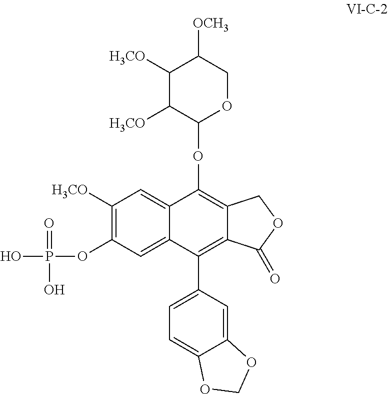 [SMILES: COc1cc2c(OC3OCC(OC)C(OC)C3OC)c3c(c(-c4ccc5c(c4)OCO5)c2cc1OP(=O)(O)O)C(=O)OC3]

or a pharmaceutically acceptable salt or prodrug thereof.

In some examples of Formula VI, the compounds are of Formula VI-D:

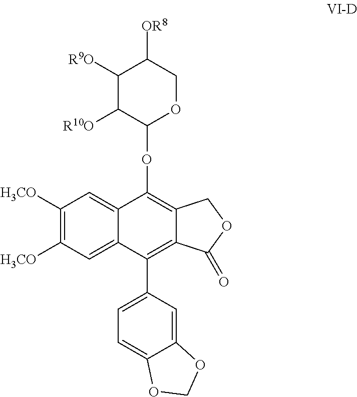

wherein

R⁸, R⁹, and R¹⁰ are independently hydrogen, halogen, substituted or unsubstituted C₁-C₄ alkyl, substituted or unsubstituted C₁-C₄ alkoxycarbonyl, hydroxycarbonyl, substituted or unsubstituted acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula VI-D, one or more of R⁸-R¹⁰ can comprise a water solubilizing group. In some examples of Formula VI-D, R⁸ and R⁹ are independently hydrogen, substituted or unsubstituted C₁-C₆ alkyl, substituted or unsubstituted C₁-C₆ acyl, or substituted or unsubstituted phosphonyl. In some examples of Formula VI-D, R⁸ and R⁹ are independently hydrogen, CH₃, C(O)CH₃, or PO₃H₂. In some examples of Formula VI-D, R⁸ and R⁹ are independently hydrogen, CH₃, or C(O)CH₃.

In some examples of Formula VI-D, the compounds are of Formula VI-E:

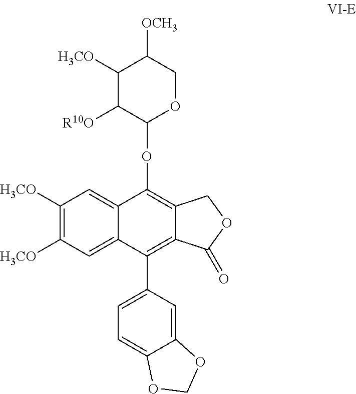

wherein

R¹⁰ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;

or a pharmaceutically acceptable salt or prodrug thereof.

In some examples of Formula VI-E, R¹⁰ is a water solubilizing group. In some examples of Formula VI-E, R¹⁰ is hydrogen, substituted or unsubstituted C₁-C₆ alkyl, substituted or unsubstituted C₁-C₆ acyl, or substituted or unsubstituted phosphonyl. In some examples of Formula VI-E, R¹⁰ is hydrogen, CH₃, C(O)CH₃, or PO₃H₂. In some examples of Formula VI-E, R¹⁰ is hydrogen, CH₃, or C(O)CH₃.

In some examples of Formula VI-E, the compound is of Formula VI-E-1:

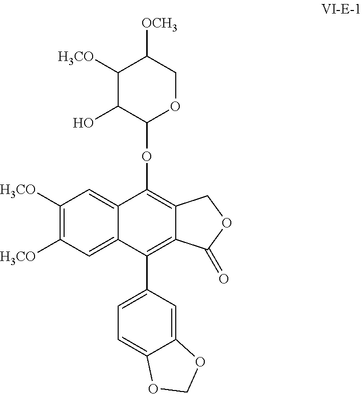 [SMILES: COc1cc2c(OC3OCC(OC)C(OC)C3O)c3c(c(-c4ccc5c(c4)OCO5)c2cc1OC)C(=O)OC3]

or a pharmaceutically acceptable salt or prodrug thereof.

In some examples of Formula VI-E, the compound is of Formula VI-E-2:

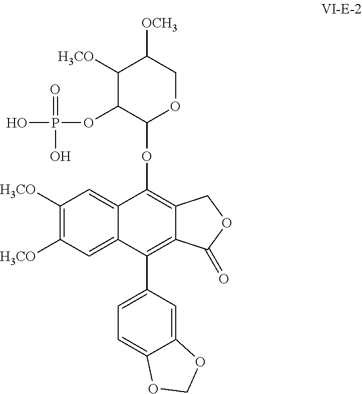 [SMILES: COc1cc2c(OC3OCC(OC)C(OC)C3OP(=O)(O)O)c3c(c(-c4ccc5c(c4)OCO5)c2cc1OC)C(=O)OC3]

or a pharmaceutically acceptable salt or prodrug thereof.

In some examples of Formula VI-D, the compounds are of Formula VI-F:

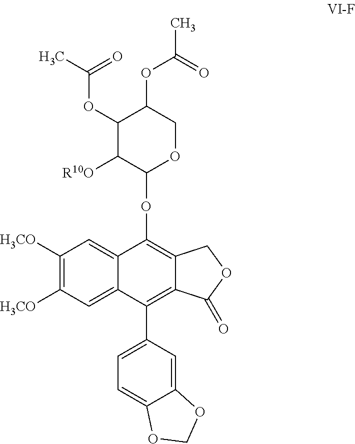

wherein

R¹⁰ is hydrogen, halogen, substituted or unsubstituted C₁-C₄ alkyl, substituted or unsubstituted alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted suilinyl, substituted or unsubstituted sulfonyl, substituted or unsubstituted sulfonamide, or substituted or unsubstituted thio;

or a pharmaceutically acceptable salt or prodrug thereof.

In some examples of Formula VI-F, R¹⁰ is a water solubilizing group. In some examples of Formula VI-F, R¹⁰ is hydrogen, substituted or unsubstituted C₁-C₆ alkyl, substituted or unsubstituted C₁-C₆ acyl, or substituted or unstibstituted phosphonyl. In some examples of Formula VI-F, R¹⁰ is hydrogen, CH₃, C(O)CH₃, or PO₃H₂. In some examples of Formula VI-F, is hydrogen, CH₃, or C(O)CH₃.

In some examples of Formula VI-F, the compound is of Formula VI-F-1:

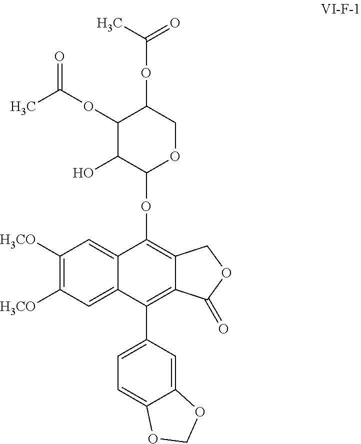 [SMILES: COc1cc2c(OC3OCC(OC(C)=O)C(OC(C)=O)C3O)c3c(c(-c4ccc5c(c4)OCO5)c2cc1OC)C(=O)OC3]

or a pharmaceutically acceptable salt or prodrug thereof.

In some examples of VI-D, one or more of R⁸-R¹⁰ can comprise a water solubilizing group in some examples of Formula VI-D, R⁸, R⁹, and R¹⁰ are independently hydrogen, substituted or unsubstituted C₁-C₆ alkyl, substituted or unsubstituted C₁-C₆ acyl, or substituted or unsubstituted phosphonyl. In some examples of Formula VI-D, R⁸, R⁹, and R¹⁰ are independently hydrogen, CH₃, C(O)CH₃, or PO₃H₂. In some examples of Formula VI-D, R⁸, R⁹, and R¹⁰ are independently hydrogen, CH₃, or C(O)CH₃.

In some examples of Formula VI-D, the compound is of Formula VI-D-1:

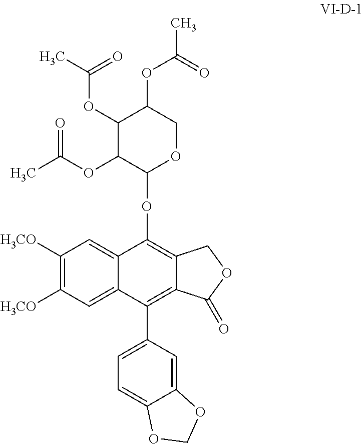 [SMILES: COc1cc2c(OC3OCC(OC(C)=O)C(OC(C)=O)C3OC(C)=O)c3c(c(-c4ccc5c(c4)OCO5)c2cc1OC)C(=O)OC3]

or a pharmaceutically acceptable salt or prodrug thereof.

In some examples of Formula VI-D, the compound is of Formula VI-D-2:

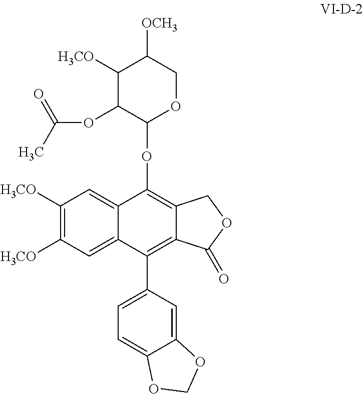 [SMILES: COc1cc2c(OC3OCC(OC)C(OC)C3OC(C)=O)c3c(c(-c4ccc5c(c4)OCO5)c2cc1OC)C(=O)OC3]

or a pharmaceutically acceptable salt or prodrug thereof.

In some examples of Formula VI-D, the compound is of Formula VI-D-3:

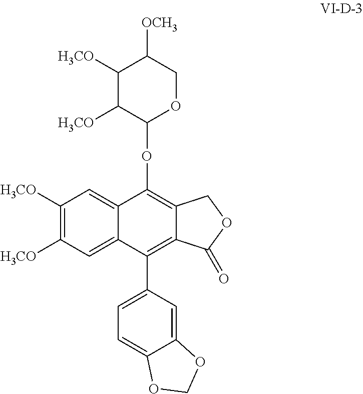 [SMILES: COc1cc2c(OC3OCC(OC)C(OC)C3OC)c3c(c(-c4ccc5c(c4)OCO5)c2cc1OC)C(=O)OC3]

or a pharmaceutically acceptable salt or prodrug thereof.

In some examples of Formula the compound is of Formula VI-D-4:

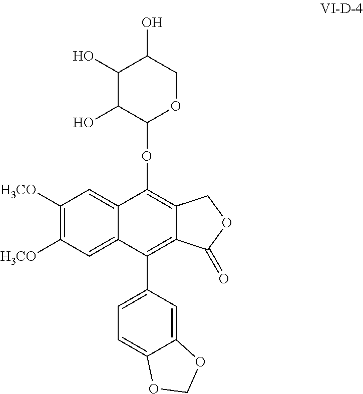 [SMILES: COc1cc2c(OC3OCC(O)C(O)C3O)c3c(c(-c4ccc5c(c4)OCO5)c2cc1OC)C(=O)OC3]

or a pharmaceutically acceptable salt or prodrug thereof.

In some examples of Formula VI-D, the compound is of Formula VI-D-5:

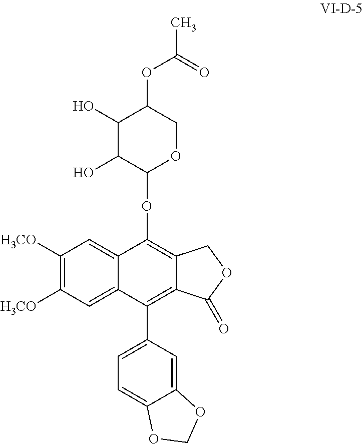 [SMILES: COc1cc2c(OC3OCC(OC(C)=O)C(O)C3O)c3c(c(-c4ccc5c(c4)OCO5)c2cc1OC)C(=O)OC3]

or a pharmaceutically acceptable salt or prodrug thereof.

In some examples of Formula VI-D, the compound is of Formula VI-D-6

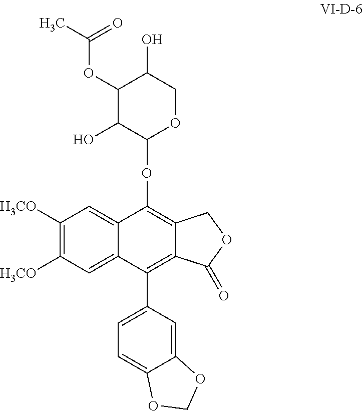 [SMILES: COc1cc2c(OC3OCC(O)C(OC(C)=O)C3O)c3c(c(-c4ccc5c(c4)OCO5)c2cc1OC)C(=O)OC3]

or a pharmaceutically acceptable salt or prodrug thereof.

### Pharmaceutical Compositions

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, prefrably in unit dosage form suitable for single administration of a precise dosage, The compositions will include a therapeutically effective amount of the compound described herein or derivatives thereof in combination with a pharmaceutically acceptable carrier and, in addition, can include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art thr use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005, Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or inmnoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene g ycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

Compositions containing the compound described herein or derivatives thereof suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions can also contain adjuvants such as preserving, wetting, emulsifying, and dispensing agents. Prevention of the action of microorganisms can be promoted by various antibacterial and antifungal agents, for example, parabens, chiorobutanot, phenol, sorbic acid, and the like. Isotonic agents, for example, sugars, sodium chloride, and the like can also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethyiceltulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as fbr example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. :In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyteneglycols, and the like.

Solid dosage forms such as tablets, dragees, capsules, pills, and granules can be prepared with coatings and shells, such as enteric coatings and others known in the art. They can contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients. The disclosed compounds can also be incorporated into polymers, examples of which include poly (D-L lactide-co-glycolide) polymer for intracranial tumors; poly[bis(pcarboxyphenoxy) propane:sebacic acid] in a 20:80 molar ratio (as used in GLIADEL); chondroitin; chitin; and chitosan.

Liquid dosage forms for oral administration of the compounds described herein or derivatives thereof include pharmaceutically acceptable emulsions, solutions, suspensions, syrups, and elixirs. In addition to the active compounds, the liquid dosage forms can contain inert diluents commonly used in the art, such as water or other solvents, soltibilizing agents, and emulsifiers, as for example, ethyl alcohol, isopropyl alcohol, ethyl carbonate, ethyl acetate, benzyl alcohol, benzyl benzoate, propyleneglycol, 1,3-butyieneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, can contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers such as cocoa butter, polyethyleneglycol or a suppository wax, which are solid at ordinary temperatures but liquid at body temperature and therefore, melt in the rectum or vaginal cavity and release the active component.

Dosage fortns for topical administration of the compounds described herein or derivatives thereof include ointments, powders, sprays, and inhalants. The compounds described herein or derivatives thereof are admixed under sterile conditions with a physiologically acceptable carrier and any preservatives, buffers, or propellants as can be required. Ophthalmic formulations, ointments, powders, and solutions are also contemplated as being within the scope of the compositions.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefitlrisk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a. suitable organic or inorganic acid and isolating the salt thus formed. Representative salts include the hydrobromide, hydrochloride, sulfate, bisulfate, nitrate, acetate, oxalate, valerate, oleate, palmitate, stearate, taurate, borate, benzoate, lactate, phosphate, tosylate, citrate, maleate, fumarate, succinate, tartrate, naphthylate mesylate, glucoheptonate, lactobionate, methane sulphonate, and lauryisulphonate salts, and the like. These can include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethytammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught herein.)

Administration of the compounds and compositions described herein or pharmaceutically acceptable salts thereof to a subject can be carried out using therapeutically effective amounts of the compounds and compositions described herein or pharmaceutically acceptable salts thereof as described herein for periods of time effective to treat a disorder.

The effective amount of the compounds and compositions described herein or pharmaceutically acceptable salts thereof as described herein can be determined by one of ordinary skill in the art and includes exemplary dosage amounts for a mammal of from about 0.5 to about 200 mg/kg of body weight of active compound per day, which can be administered in a single dose or in the fbrm of individual divided doses, such as from 1 to 4 times per day. Alternatively, the dosage amount can be from about 0.5 to about 150 mg/kg of body weight of active compound per day, about 0.5 to 100 mg/kg of body weight of active compound per day, about 0.5 to about 75 mg/kg of body weight of active compound per day, about 0.5 to about 50 mgikg of body weight of active compound per day, about 0.5 to about 25 mg/kg of body weight of active compound per day, about 1 to about 20 mg/kg of body weight of active compound per day, about 1 to about 10 mg/kg of body weight of active compound per day, about 20 mg/kg of body weight of active compound per day about 10 mg/kg of body weight of active compound per day, or about mg/kg of body weight of active compound per day. The expression effiNtive amount, when used to describe an amount of compound in a method, refers to the amount of a compound that achieves the desired pharmacological effect or other effect, for example an amount that results in enzyme

Those of skill in the art will understand that the specific dose level and frequency of dosage for any particular subject can be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

### Methods of Making the Compounds

The compounds described herein can be prepared in a variety of ways known to one skilled in the art of organic synthesis or variations thereon as appreciated by those skilled in the art. The compounds described herein can be prepared from readily available starting materials. Optimum reaction conditions can vary with the particular reactants or solvents used, but such conditions can be determined by one Mcilled in the art.

Variations on the compounds discussed herein include the addition, subtraction, or movement of the various constituents as described for each compound. Similarly, when one or more chiral centers are present in a molecule, the chirality of the molecule can be changed. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts and Greene, Protective Groups in Organic Synthesis, 4th Ed,, Wiley & Sons, 2006, which is incorporated herein by reference in its entirety.

The starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), Sigma (St. Louis, Mo.), Pfizer (New York, N.Y.), CilaxoSmithKline (Raleigh, N.C.), Merck (Whitehouse Station, N.J.), Johnson & Johnson (New Brunswick, N.J.), Aventis (Bridgewater, N. J.), AstraZeneca (Wilmington, Del.), Novartis (Basel, Switzerland), Wyeth (Madison, N. J.). Bristol-Myers-Squibb (New York, N.Y.), Roche (Basel, Switzerland), Lilly (Indianapolis, Ind.), Abbott (Abbott Park, Ill.), Schering Plough (Kenilworth, N.J.), or Boehringer Ingelheim (Ingelheim, Germany), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989). Other materials, such as the pharmaceutical carriers disclosed herein can be obtained from commercial sources.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

### Activity Assays

The activity of the compounds provided herein as anticancer and immunostimulatory agents can be measured in standard assays. The activities of the compounds as determined using the assays described herein can be reported in terms of IC₅₀. As used herein, IC₅₀ refers to an amount, concentration, or dosage of a particular test compound that achieves a 50% inhibition of a maximal response in an assay that measures such response.

In certain aspects, the disclosed compounds and compositions need not actually be synthesized, but instead can be used as targets for any molecular modeling technique to predict and characterize interactions with cancer associated enzymes. This is achieved through structural information and computer modeling. Computer modeling technology allows visualization of the three-dimensional atomic structure of a selected molecule and the rational design of new compounds that will interact with an enzyme. The three-dimensional construct of the enzyme typically depends on data from x-ray crystallographic analyses or NMR imaging of the selected molecule. The molecular dynamics require force field data (e.g., Merck Molecular Force Field). The computer graphics systems enable prediction of how a new compound will link to the enzyme and allow experimental manipulation of the structures of the compound to perfect binding specificity. Prediction of what the interactions will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA pc.nforms the construction, graphic modeling and analysis of molecular structure. QUANTA allows interactive construction, modification, visualization, and analysis of the behavior of molecules with each other. Upon identification of compounds that interact in a desired way with the enzyme in silico, actual compounds can be synthesized and assayed as disclosed herein.

### Kits

Also provided herein are kits for treating or preventing cancer in a subject. A kit can include any of the compounds or compositions described herein. A kit can further include one or more anti-cancer agents (e.g., pactitaxel). A kit can include an oral formulation of any of the compounds or compositions described herein. A kit can additionally include directions for use of the kit (e.g., instructions for treating a subject).

The examples below are intended to further illustrate certain aspects of the methods and compounds described herein, and are not intended to limit the scope of the claims.

### Methods of Use

Provided herein are methods of treating, preventing, or ameliorating cancer in a subject. Also provided are methods of stimulating the immune system of a subject. These methods include administering to a subject an effective amount of one or more of the compounds or compositions described herein, or a phatmaceutically acceptable salt or prodrug thereof. The compounds and compositions described herein or pharmaceutically acceptable salts thereof are useful for treating cancer in humans, e.g., pediatric and geriatric populations, and in animals, e.g., veterinary applications. They can also be useful as immunostimulants. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall is bladder, kidney, mouth, larynx, esophagus, stomach, testis, cervix, mesothelioma, neuroendocrine, penis, skin, spinal cord, thyroid, vagina, vulva, uterus, liver, muscle, blood cells (i)cluding lymphocytes and other immune system cells). Some examples of cancers contemplated for treatment include carcinomas, Karposi's sarcoma, melanoma, mesothelioma, soft tissue sarcoma, pancreatic cancer, colon cancer, lung cancer, leukemia (acute lymphoblastic, acute myeloid, chronic lymphocytic, chronic myeloid, and other), and lymphoma (Burkitt's, follicular, Hodgkin's, non-Hodgkin's, mantle cell, and other), and multiple myeloma.

The methods of treatment or prevention described herein can further include treatment with one or more additional agents (e.g., an anticancer agent or ionizing radiation). The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts or prodrugs thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-Fluorouracil, 6-Thioguanine, 6-Mercaptopurine, Accutane, Actinomycin-D, Adriamycin, Adrucil, Agrylin, Ada-Cort, Aldesleukin, AlemtuzumabAlitretinoin, Alkaban-AQ, Alkeran, All-trans-retinoic acid, Alpha-interferon, Altretamine, Amethopterin, Amifostine, Aminoglutethimide, Anagrelide, Anandron, Anastrozole, Arabinosylcytosine, Aranesp, ArediaArimidex, Aromasin, Arsenic trioxide, Asparaginase, ATRA, Avastin, BCG, BCNU, Bevacizumab, Bexarotene, Bicalutamide, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, Cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Datinortibicin, Daunorubicin hydrochloride, Daunorubicin liposomal, DaunoXome, Decadron, Delta-Cortef, Dettasone, Deniteukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eilence, Eloxatin, Elspar, Emcyt, EpirUbicin, Epoetin alfa, Erbitux, Erwinia L-asparaginase, Estramustine, Ethyol, Etopophos, Etoposide, Etoposide phosphate, Eutexin, Evista, Exemestane, Fareston, Faslodex, Femafa, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gieevec, Lupron, Lupron Depot, Matulane, Maxidex, Mechlorethamine, -Mechlorethamine Hydrochlorine, Medralone, Medrol, Megace, Megestrol, IMegestroi Acetate, Melphalan, Mercaptopurine, Mesna, Mesnex, Methotrexate, Mefhotrexate Sodium, Methylprednisolone, Mylocel, Letrozole,

Neosar, Neulasta, Neumega, Neupogen, Nitandron, Nilutatnide, Nitrogen Mustard, Novaidex, Novantrone, Octreotide, Octreotide acetate, Oncospar, Oncovin, Ontak, Onxal, Oprevelkin, Orapred, Orasone, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interfron alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumoinab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, interferon Alfa-2b (PEG conjugate), Interleukin 2, Interteukin-11, Intron A (Interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitornycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-Asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of killing a tumor cell in a subject. The method includes contacting the tumor cell with an effective amount of a compound or composition as described herein, and optionally includes the step of irradiating the tumor cell with an effective amount of ionizing radiation. Additionally, methods of radiotherapy of tumors are provided herein. The methods include contacting the tumor cell with an effective amount of a compound or composition as described herein, and irradiating the tumor with an effective amount of ionizing radiation. As used herein, the term ionizing radiation refers to radiation comprising particles or photons that have sufficient energy or can produce sufficient energy via nuclear interactions to produce ionization. An example of ionizing radiation is X-radiation. An effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms of an infection. Prophylactic administration can be used, fbr example, in the chemopreventative treatment of subjects presenting precancerous lesions, those diagnosed with early stage malignancies, and for subgroups with susceptibilities (e.g., family, racial, and/or occupational) to particular cancers. Therapeutic treatment involves administering to a subject a therapeutically effective amount of the compounds and compositions or pharmaceutically acceptable salts thereof as described herein after cancer is diagnosed.

In some examples, the compounds disclosed herein are not topoisomerase II inhibitors. In some examples, the compounds disclosed herein can activate caspase-3.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to nutribers (e.g., amounts, temperature, etc.) but some errors and deviations should be accounted for. Unless indicated otherwise, parts are parts by weight, temperature is ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

The melting point was measured using a Fisher Scientific apparatus and is uncorrected, Specific rotation values were obtained on a Perkin-Elmer model 343 polarimeter. UV spectra were recorded on a Hitachi U2910 LTV spectrophotometer. ECD measurements were performed using a JASCO J-810 spectropolarimeter, IR spectra were recorded on a Nicolet 6700 FT-IR spectrometer. ¹H and ¹³C, DEPT, HSQC, HMBC, NOESY, and COSY NMR spectra were recorded at room temperature on Bruker Avance DRX-400, DRX-600, or DRX-800 MHz NMR spectrometers. ESIMS and HRESIMS were measured on a LCT-TOE or a Q-TOF mass spectrometer in the positive-ion mode. Column chromatography was conducted using silica gel (65×250 or 230×400 mesh, Sorbent Technologies, Atlanta, Ga.). Analytical thin-layer chromatography (TLC) was performed on precoated silica gel 60 F254 plates (Sorbent Technologies, Atlanta, Ga.), Sephadex LH-20 was purchased from Amersham Biosciences, Uppsala, Sweden. For visualization of TLC plates, sulfuric acid reagent was used. Fluorescence was tested using a Spectroline (model ENF-260C) UV light source. All procedures were carried out using anhydrous solvents purchased from commercial sources and employed without further purification. Reagents for chemical synthesis were purchased from Sigma except where indicated, and reactions were monitored by TLC using precoated silica gel plates. Crystallographic data were collected through the Service Crystallography at Advanced Light Source (SCrALS) program at the Small-Crystal Crystallography Beamline 11.3.1 at the Advanced Light Source (ALS), Lawrence Berkeley National Laboratory, with Bruker APEXII CCD detector (Bruker Analytical X-ray Instruments, Inc., Madison, Wis.).

### Example 1

Six aryl naphthalene lignan lactones (1-6) were isolated from different plant parts of Phyllanthus poilanei collected in Vietnam, with two further analogues (7 and 8) being prepared from phyllanthustnin C (4) Phyllanthus is a large plant genus containing over 600 species (Lin M T et al. J. Nat. Prod. 1995, 58, 244-249; Tuchinda P et al. Planta Med. 2006, 72, 60-62; Wu S J and Wu T S. Chem. Pharm. Bull. 2006, 54, 1223-1225; Tuchinda P et al. J. Nat. Prod 2008, 71, 655-663; Wang C Y et al. Phytoehem. Anal. 2011, 22, 352-360). As part of a search for anticancer agents from plants and other organisms (Kinghorn A D et al. Pure Appl. Chem. 2009, 81, 1051-1063), an initial crude chloroform-soluble extract of Phyllanthus poilanei Beale collected in Vietnam was found to exhibit cytotoxicity toward the HT-29 human colon cancer cell line.

Plant Material. Initial collections of separate samples of the combined leaves, twigs, flowers, and fruits (acquisition number A06024) and the stems (acquisition number A06025) of Phyllanthus poilanei were collected from a shrub at the road transect from Suoi Cat village to Hon Ba peak (12° 07.873′ N; 106° 01.532′ E), Dinh Khanh District, Khanh Hoa Province, Vietnam, in November, 2004. A voucher herbarium specimen (DDS 13619) representing this collection was deposited at the John G. Searle Herbarium of the Field Museum of Natural Histoiy, Chicago, Ill., under the accession number FM-2256257.

Second collections of separate samples of the combined leaves, twigs, flowers, and fruits (acquisition number A06473) and the stems (acquisition number A06474) of P. poilanei were obtained from a liana-like shrub at the forest occurring at the south end of Kego Lake, across from Mui Tru Ranger Station (18° 06.530′ N; 106° 00.891′ E), Kego Nature Reserve, Cam Xuyen District, Hatinh Province, Vietnam, in December, 2008. A voucher herbarium specimen (DDS 14308) representing this collection was deposited at the John G. Searle Herbarium of the Field Museum of Natural History, Chicago, Ill., under the accession number FM-2287526.

A larger sample of the combined leaves, twigs, and stems (acquisition number AA06024) of P. poilanei was collected from a liana in the Hon Ba mountain region, 2.5 km from Soi Cat on a peak along roadside forest (12° 06.745′ N; 108° 58.80′ E), Dinh Khanh District, Khan Hoa Province, Vietnam, in August, 2011. A voucher herbarium specimen (DDS 14886) representing this collection was deposited at the John G. Searle Herbarium of the Field Museum of Natural History, Chicago, Ill., under the accession number FM-2300873.

Extraction and Isolation. The milled air-dried leaves, twigs, flowers, and fruits of P. poilanel (sample A06024, 2000 g) were extracted with MeOH (7 L×6) at room temperature. The solvent was evaporated in vacua, and the dried MeOH extract (170 g, 8.5%) was resuspended in 10% H₂O in MeOH (1000 mL) and partitioned with n-hexane (700 mL×2 and 500 mL) to yield a n-hexane-sollible residue (D1, 22.4 g, 1.1%). The aqueous MeOH layer was then partitioned with CHCl₃ (800, 700, and 600 mL) to afford a chloroform-soluble extract (D2, 3.0 g, 0.15%), which was washed with a 1% aqueous solution of NaCl, to partially remove tannins The chloroform-soluble extract exhibited cytotoxicity toward the HT-29 cell line (IC₅₀<5.0 μg/mL). Both the n-hexane- and aqueous-soluble extracts were inactive in the bioassay system used. The chloroform-soluble extract (2.8 g) was subjected to silica gel column chromatography (2.5×45 cm) and eluted with a gradient of n-hexane-acetone. Eluates were pooled by TLC analysis to give thirteen combined fractions (D2F1-D2F13). Of these, D2F4-D2F6 (IC₅₀<2 μg/mL) were combined and further chromatographed over a silica gel column (2.5×20 cm), eluted with a gradient of n-hexane-acetone to yield seven pooled subtractions (D2F4F1-D2F4F7). D2F11 and D2F12 (IC₅₀<5 μg/mL) were combined and further chromatographed over a silica gel column (2.5×20 cm), eluted with a gradient of n-hexane-acetone, to yield five combined subfractions (D2F11F1-D2F11F5), Subfraction D2F4F2 was chromatographed over silica gel, with a gradient of n-hexane-acetone, and then purified by separation over a Sephadex LH-20 column, eluted with CH₂Cl₂-MeOH (1:1), affording phyllanthusmin D (1, 20 rag). The combined suhfractions D2F4F3-D2F4F5 were separated by silica gel chromatography, eluted with n-hexane-acetone (3:1), and then purified by passage over a Sephadex LH-20 column, eluted with a mixture of CH₂Cl₂-MeOH (1:1), to afford phyllanthusmin A (6, 2.0 mg), phyilanthusmin B (3, 1.0 mg), and phyllanthusmin E (2, 1.5 mg). Fractions D2F11F2-D2F11F4 were combined and chrornatographed over silica gel, eluted by n-hexane-acetone (2:1), and then purified by separation over a Sephadex LH-20 column, using CH₂Cl₂-MeOH (1:1) for elution, affording phyllanthusmin C (4, 7.0 mg).

The milled air-dried stems of P. poilanei (sample A06025, 580 g) were extracted with MeOH (3 L×4 and then 2 L×2) at room temperature. The solvent was evaporated in vacuo, and the dried MeOH extract (47.4 8.2%) was resuspended in 10% H₂O in MeOH (500 mL) and partitioned with n-hexane (500, 300, 200 mL), to yield a n-hexane-soitibie residue (D1, 1.4 g, 0,24%), The aqueous MeOH layer was then partitioned with CHCl₃ (500, 300, and 300 mL) to afford a chloroform-soluble extract (D2, 2.0 g, 0.34%), which was followed by washing with a 1% aqueous solution of NaCl, to partially remove tannins. The chloroform-soluble extract exhibited cytotoxicity toward the HT-29 cell line (IC₅₀<5.0 μg/mL). Both the n-hexane- and aqueous-soluble extracts were inactive in the bioassay system used. The chloroform-soluble extract (1.8 g) was subjected to silica gel column chromatography (2.5×45 cm) and eluted with a gradient of n-hexane-acetone. Fractions were pooled by TLC analysis to give thirteen combined fractions (D2F1-D2F13). Of these, D2F4-D2F6 (IC₅₀<2 μg/mL) were combined and further chromatographed over a silica gel column, eluted with a gradient of n-hexane-acetone and then purified by separation over a Sephadex LH-20 column, eluted with CH₂Cl₂-MeOH (1:1), affording phyllanthusmin D (2, 7.0 mg).

The milled air-dried combined leaves, twigs, flowers, and fruits of P. poilanei (sample A06473, 851 g) were extracted with MeOH (3 L×4, 2 L×2) at room temperature. The solvent was evaporated in vacuo, and the dried MeOH extract (96 g, 11.3%) was resuspended in 10% H₂O in MeOH (500 mL) and partitioned with n-hexane (500, 300, 200 mL), to yield a n-hexane-soluble residue (D1, 10,2 g, 1.2%). The aqueous MeOH layer was then partitioned with CHCl₃ (500, 300, and 300 mL) to afford a chloroform-soluble extract (D2, 3.0 g, 0.35%), which was followed by washing with a 1% aqueous solution of NaCl, to partially remove tannins. The chloroform-soluble extract exhibited cytotoxicity toward the HT-29 cell line (IC₅₀<10.0 μg/mL). Both the n-hexane- and aqueous-soluble extracts were inactive in the bioassay system used. The chloroform-soluble extract (2.8 g) was subjected to silica gel column chromatography (2.5×45 cm) and eluted with a gradient of n-hexane-acetone. Fractions were pooled by TLC analysis to give eleven combined fractions (D2F1-D2F11). Of these, D2F8 and D2F9 (IC50 <5.0₁,tg/mL) were combined and further chromatographed over a silica gel column (2.5×20 cm), eluted with a gradient of n-hexane-acetone and then purified by separation over a Sephadex LH-20 column, eluted with CH₂Cl₂-MeOH (1:1), affording phyllanthusmins C (4, 2.0 mg) and D (1, 3.0 mg).

The milled air-dried stems of P. poilanei (sample A06474, 517 g) were extracted with MeOH (2 L×6) at room temperature. The solvent was evaporated in vacuo, and the dried MeOH extract (75 g, 14.5%) was resuspended in 10% H₂O in MeOH (600 mL) and partitioned with n-hexane (500, 400, and then 300 mL), to yield a n-hexane-soluble residue (D1, 1.0 g, 0,2%), The aqueous MeOH layer was then partitioned with CHCl₃ (500, 300, and 300 mL) to afford a chloroform-soluble extract (D2, 2.0 g, 0.38%), which was followed by washing with a 1% aqueous solution of NaCl, to partially remove tannins. The chloroform-soluble extract exhibited cytotoxicity towards the HT-29 cell line (IC₅₀<10.0 μg/mL). Both the n-hexane- and aqueous-soluble extracts were inactive in the bioassay system used. The chloroform-soluble extract (1.8 g) was subjected to silica gel column chromatography (2.5×45 cm) and eluted with a gradient of n-hexane-acetone. Fractions were pooled by TLC analysis to give eleven combined fractions (D2F1-D2F11). Of these, D2F10 (IC₅₀<5.0 μg/mL) was chromatographed over a silica gel column, and eluted with a gradient of n-hexane-acetone and then purified by separation over a Sephadex LH-20 column, eluted with CH₂Cl₂-MeOH (1:1), affording phyllanthusmin D (1, 2.0 mg).

In an attempt to accumulate a larger quantity of the isolate phyllanthusmin D (1) for in vivo biological evaluation, a larger recollection of the combined leaves, twigs, and sterns of P. poilanei was made. The milled air-dried combined leaves, twigs, and stems of this sample (AA06024, 3200 g) were extracted with MeOH (7 L×6) at room temperature. The solvent was evaporated in vacuo, and the dried MeOH extract (278.0 g, 8.7%) was resuspended in 10% H₂O in MeOH (1000 mL) and partitioned with n-hexane (800 mL×3 and 500 mL×3), to yield a n-hexane-soluble residue (D1 , 27.0 g, 0.84%). The aqueous MeOH layer was then partitioned with CHCl₃ (800 mL×3 and 500 mL×3) to afford a chloroform-soluble extract (D2, 8.5 g, 0.27%), which was followed by washing with a 1% aqueous solution of NaCl, to partially remove tannins. The aqueous MeOH layer was further partitioned with EtOAc (800 mL×3 and 500 mL×3) to afford an EtOAc-soluble extract (D3, 10.0 g, 0.31%), which was also washed with a 1% aqueous solution of NaCl. The chloroform-soluble extract exhibited cytotoxicity towards the HT-29 cell line (IC₅₀<5.0 μg/mL). However, all of the n-hexane-, EtOAc-, and aqueous-soluble extracts were inactive in the bioassay system used. The chloroform-soluble extract (8.0 g) was subjected to silica gel column chromatography (4.5×45 cm) and eluted with a gradient of n-hexane-acetone. Fractions were pooled by TLC analysis to give eleven combined fractions (D2F1-D2F11). Of these, D2F4-D2F6 (IC₅₀<5 μg/mL) were combined and further chrornatographed over a silica gel column (2.5×20 cm), eluted with a gradient of n-hexane-acetone to yield phyllanthusmins B (3, 1.0 mg), D (1, 10.5 mg), and E (2, 1.0 mg) Fraction D2F8 was separated by silica gel chromatography, eluted with n-hexane-acetone (2:1), and then purified by passage over a Sephadex LH-20 column, eluted with a mixture of CH₂Cl₂-MeOH (1:1), to afford phyilanthusmin C (4, 9.5 mg). To isolate the polar analogues of 4, the EtOAc-soluble extract (9.0 g) was subjected to silica gel column chromatography (4.5×45 cm) and eluted with a gradient of CH₂Cl₂-MeOH. Fractions were pooled by TLC analysis to give five combined fractions (D3F1-D3F5). Of these, D3F1 and D3F2 were combined and further chromatographed over a silica gel column (2.5×20 cm), eluted with a gradient of CH₂Cl₂-MeOH, then purified by passage over a Sephadex LH-20 column, eluted with a mixture of CH₂Cl₂-MeOH (1:1), to afford cieistanthin B (5, 1.5 mg).

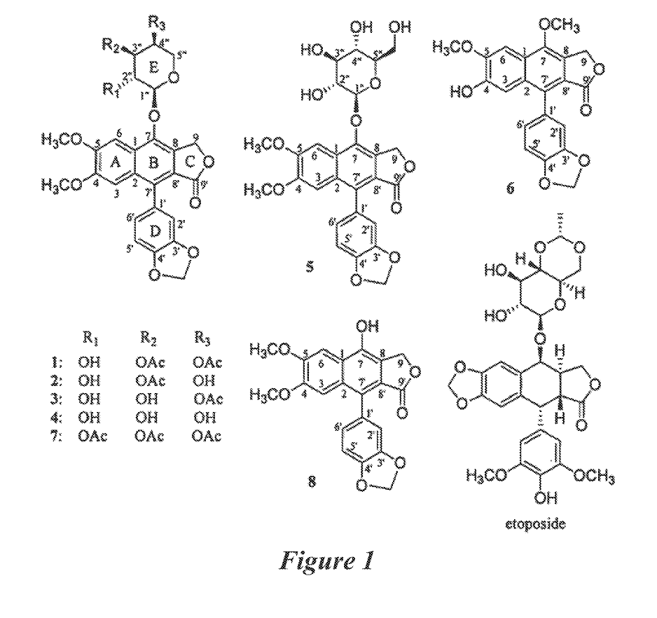

The structures of compounds 1-8 and etoposide are illustrated in (FIG. 1).

The structures of the compounds 1 and 2 were determined by interpretation of their spectroscopic data and by chemical methods, and the structure of phylianthusmin D (1) was confirmed by single-crystal X-ray diffraction analysis. Several of these arylnaphthalene tignan lactones were cytotoxic toward HT-29 human colon cancer cells, with compounds 1 and 7-O-((2,3,4-tri-O-acetyl)-α-L-arabinopyanosyl) diphyltin (7) found to be the most potent, exhibiting IC₅₀ values of 170 and 110 nM, respectively. Compound 1 showed activity when tested in an in vivo hollow fiber assay using HT-29 cells implanted in immunodeficient NCr nu/nu mice. Mechanistic studies showed that this compound mediated its cytotoxic effects by inducing tumor cell apoptosis through activation of caspase-3, but it did not inhibit DNA topoisomerase IIα activity.

Phyllanthusmin D (1): Colorless fine needles (n-hexane/acetone), showing a blue color under UV light at 365 nm; mp 210-211° C.; [α]²⁰_{D} −3.3 (c 0.09, CHCl₃); tiV (MeOH) λₘₐₓ (log ε) 260 (4.54) nm; ECD (MeOH, nm) λₘₐₓ (Δε) 292 (-3.65); IR (dried film) vₘₐₓ 3446, 1747, 1619, 1507, 1481, 767 cm⁻¹; positive-ion ITRESIMS nn/z 619.1444, calcd for C₃₀H₂₈O₁₃Na, 619.1428.

Phyllanthusmin E (2): Amorphous colorless powder showing a blue color under UV light at 365 nm; [α]²⁰_{D} −4.4 (c 0.09, CHCl₃); UV (MeOH) λₘₐₓ (log ε) 260 (4.58) nm; ECD (MeOH, n,) λₘₐₓ (Δε) 296 (−4.15); IR (dried film) vₘₐₓ 3419, 1738, 1622, 1506, 1481, 770 cm⁻¹; positive-ion HRESIMS m/z 577.1319, calcd for C₂₈H₂₆O₁₂Na, 577.1322.

TABLE 1
¹H NMR and ¹³C NMR Spectroscopic Data of Compounds 1 and 2^{a}.

| | Compound 1 | | Compound 2 | |
|---|---|---|---|---|
| position | δ_{C},^{b} type | δ_{H},^{c} (J in Hz) | δ_{C},^{d} type | δ_{H},^{e} (J in Hz) |
| 1 | 127.1 C | | 127.2 C | |
| 2 | 131.0 C | | 131.0 C | |
| 3 | 106.4 CH | 7.09 d (2.0^{f}) | 106.4 CH | 7.10 s |
| 4 | 150.3 C | | 150.4 C | |
| 5 | 152.2 C | | 152.2 C | |
| 6 | 100.8 CH | 7.94 s | 100.9 CH | 7.95 s |
| 7 | 144.2 C | | 144.3 C | |
| 8 | 131.4^{g} C | | 131.4 C | |
| 9 | 67.6 CH₂ | 5.47 d (15.2) | 67.6 CH₂ | 5.46 ddd (9.6, |
| | | 5.56 d (15.2) | | 2.4, 1.2^{f}) |
| | | | | 5.57 ddd (11.4, |
| | | | | 3.6, 1.8^{f}) |
| 1′ | 128.4 C | | 128.4 C | |
| 2′ | 110.8 CH | 6.83 overlapped | 110.9 CH | 6.84 d (0.6^{f}) |
| 3′ | 147.7 C | | 147.7 C | |
| 4′ | 147.7 C | | 147.7 C | |
| 5′ | 108.4 CH | 6.97 d (8.0) | 108.4 CH | 6.97 dd (5.4, 1.2^{f}) |
| 6′ | 123.7 CH | 6.81 overlapped | 123.8 CH | 6.82 dd (6.0, 1.2^{f}) |
| 7′ | 136.9 C | | 136.9 C | |
| 8′ | 119.4 C | | 119.4 C | |
| 9′ | 170.0 C | | 169.9 C | |
| 1″ | 105.4 C | 4.86 d (7.6) | 105.7 CH | 4.84 d (6.0) |
| 2″ | 70.0 CH | 4.31 t (8.8) | 70.3 CH | 4.33 t (6.6) |
| 3″ | 73.3 CH | 4.99 dd | 75.9 CH | 4.92 br d (7.8) |
| | | (10.0, 3.6) | | |
| 4″ | 68.1 CH | 5.30 br s | 67.2 CH | 4.12 m |
| 5″ | 64.9 CH₂ | 3.60 d (13.2) | 66.7 CH₂ | 3.56 d (12.3) |
| | | 4.06 overlapped | | 4.09 d (11.4) |
| OMe-4 | 56.0 CH₃ | 3.81 s | 56.0 CH₃ | 3.81 s |
| OMe-5 | 56.5 CH₃ | 4.03 s | 56.5 CH₃ | 4.03 s |
| OCH₂O- | 101.4 CH₂ | 6.05 s | 101.4 CH₂ | 6.05 s |
| 3′,4′ | | 6.10 s | | 6.10 s |
| OAc-3″ | 170.8 C | | 171.2 C | |
| | 21.1 CH₃ | 2.14 s | 21.3 CH₃ | 2.25 s |
| OAc-4″ | 170.4 C | | | |
| | 21.0 CH₃ | 2.23 s | | |

^{a}Assignments of chemical shifts are based on the analysis of 1D- and 2D-NMR spectra. The overlapped signals were assigned from ¹H-¹H COSY, HSQC, and HMBC spectra without designating multiplicity. CH₃, CH₂, CH, and C multiplicities were determined by DEPT 90, DEPT 135, and HSQC experiments.
^{b}Data (δ) measured at 100.6 MHz and referenced to residual CDCl₃ at δ 77.16.
^{c}Data (δ) measured at 400.1 MHz and referenced to residual CDCl₃ at δ 7.26.
^{d}Data (δ) measured at 150.9 MHz and referenced to residual CDCl₃ at δ 77.16.
^{e}Data (δ) measured at 600.2 MHz and referenced to residual CDCl₃ at δ 7.26.
^{f}The unusual value may result from the restricted rotation of the D ring.
^{g}Present in pairs at room temperature (131.43/131.42).

Phyllanthusmin B (3): Amorphous colorless powder showing a blue color under UV light at 365 nm; [α]²⁰_{D} −6.0 (c 0.05, CHCl₃); UV (MeOH) λₘₐₓ (log ε) 260 (4.62) nm; ECD (MeOH, nm) λₘₐₓ (Δε) 297 (−4.12); IR (dried film) vₘₐₓ 3364, 1723, 1615, 1505, 1480, 765 cm⁻¹; positive-ion HRESIMS m/z 577,1317, calcd for C₂₈H₂₆O₁₂Na, 577.1322.

Phyllanthusmin C (4): Amorphous colorless powder showing a blue color under UV light at 365 nm; [α]²⁰_{D} −8.0 (c 0.06, CHCl₃); UV (MeOH) λₘₐₓ (log ε) 260 (4.35) nm; CD (MeOH, nm) λₘₐₓ (Δε) 292 (−3.19); IR (dried film) vₘₐₓ 3373,1734, 1619, 1507, 1480, 767 cm⁻¹; positive-ion HRESIMS m/z 535.1237, calcd fbr C₂₆H₂₄O₁₁Na, 535.1216.

Cleistanthin B (5): Amorphous colorless powder showing a blue color under UV light at 365 nm; [α]²⁰_{D} −53.3 (c 0.06, MeOH); UV (MeOH) λₘₐₓ (log ε) 260 (4.73) nm, CD (MeOH, nm) λₘₐₓ (Δε) 301 (−4.94); IR (dried film) vₘₐₓ 3390, 1739, 1713, 1622, 1506, 1481, 770 cm⁻¹; positive-ion HRESIMS iniz 565.1321, calcd for C₂₁H₂₆O₁₂Na, 565.1322.

TABLE 2
¹H NMR Spectroscopic Data of Compounds 3-5^{a}.

| position | 3^{b} | 4^{c} | 5^{d} |
|---|---|---|---|
| 3 | 7.00 s | 6.98 br s | 7.11 s |
| 6 | 8.17 s | 8.18 br s | 8.28 s |
| 9 | 5.40 d (8.4^{e}) | 5.47 d (15.2) | 5.45 dd (15.0, 3.0^{e}) |
| | 5.51 d (7.8^{e}) | 5.55 d (15.2) | 5.77 dd (15.0, 3.0^{e}) |
| 2′ | 6.94 s | 6.93 br s | 6.90 dd (15.6^{e}, 1.2) |
| 5′ | 7.06 d (12.0^{e}) | 7.05 d (8.0) | 6.99 dd (7.8, 1.8) |
| 6′ | 6.81 d (12.0^{e}) | 6.81 dd (8.0, 1.6) | 6.85 m |
| 1″ | 4.84 t (10.2) | 4.81 t (6.8) | 4.95 d (7.8) |
| 2″ | 3.88 br d (19.2^{e}) | 3.86 m | 3.71 m |
| 3″ | 3.80 m | 3.52 m | 3.56 m |
| 4″ | 4.99 br s | 3.71 br s | 3.50 m |
| 5″ₐₓ | 3.68 overlapped | 3.46 d (11.74) | 3.42 m |
| 5″_{eq} | 3.96 overlapped | 3.79 m | |
| 6″ | | | 3.85 m |
| | | | 3.97 br d (10.2) |
| MeO-4 | 3.68 overlapped | 3.67 s | 3.74 s |
| MeO-5 | 3.96 overlapped | 3.95 s | 4.01 s |
| OCH₂O-3′,4′ | 6.14 s | 6.13 br s | 6.09 s |
| | 6.14 s | | 6.10 s |
| AcO-4″ | 2.12 s | | |

^{a}Chemical shifts were assigned based on the analysis of 1D- and 2D-NMR spectra. The overlapped signals were assigned from ¹H-¹H COSY, HSQC, and HMBC spectra without designating multiplicity (s = singlet, br s = broad singlet, d = doublet, br d = broad doublet, dd = double doublet, dt = double triplet, m = multiple). Proton coupling constant J (in parentheses) values are presented in Hz and were omitted if the signals overlapped as multiplets.
^{b}Data (δ) recorded at 600.2 MHz in DMSO-d₆ and referenced to residual DMSO-d₆ at δ 2.50.
^{c}Data (δ) recorded at 400.1 MHz in DMSO-d₆ and referenced to residual DMSO-d₆ at δ 2.50.
^{d}Data (δ) measured at 400.1 MHz in acetone-d₆ and referenced to residual acetone-d₆ at δ 2.05.
^{e}The unusual value may result from the rotation conformation of the D ring.

TABLE 3
¹³C NMR Spectroscopic Data of Compounds 3-5^{a}.

| position | 3^{b} | 4^{c} | 5^{d} |
|---|---|---|---|
| 1 | 126.6 C | 126.6 C | 128.2 C |
| 2 | 129.8 C | 129.7 C | 131.3 C |
| 3 | 105.5 CH | 105.4 CH | 106.6 CH |
| 4 | 150.0 C | 150.0 C | 151.4 C |
| 5 | 151.5 C | 151.4 C | 153.0 C |
| 6 | 101.7 CH | 101.9 CH | 102.7 CH |
| 7 | 144.7 C | 144.6 C | 146.15/146.14^{f} C |
| 8 | 129.4 C | 128.98/128.81^{f} C | 131.59/131.56^{f} C |
| 9 | 67.0 CH₂ | 67.1 CH₂ | 68.1 CH₂ |
| 1′ | 128.2 C | 128.3 C | 129.8 C |
| 2′ | 110.86/110.76^{f} CH | 110.91/110.83^{f} CH | 111.76/111.69^{f} |
| | | | CH |
| 3′ | 146.9 C | 146.9 C | 148.3 C |
| 4′ | 146.9 C | 146.9 C | 148.2 C |
| 5′ | 108.0 CH | 108.0 CH | 108.7 CH |
| 6′ | 123.59/123.55^{f} CH | 123.6 CH | 124.5 CH |
| 7′ | 134.9 C | 134.55/134.50^{f} C | 136.4 C |
| 8′ | 118.7 C | 118.7 C | 120.1 C |
| 9′ | 169.0 C | 169.1 C | 169.9 C |
| 1″ | 105.14/105.06^{f} CH | 104.90/104.78^{f} CH | 106.3 CH |
| 2″ | 71.2 CH | 70.8 CH | 75.2 CH |
| 3″ | 70.4 CH | 72.3 CH | 78.1 CH |
| 4″ | 70.7 CH | 67.32/67.27^{f} CH | 71.4 CH |
| 5″ | 63.7 CH₂ | 65.79/65.73^{f} CH₂ | 78.2 CH |
| 6″ | | | 62.8 CH₂ |
| MeO-4 | 55.2 CH₃ | 55.2CH₃ | 55.7 CH₃ |
| MeO-5 | 55.8 CH₃ | 55.9 CH₃ | 56.4 CH₃ |
| OCH₂O- | 101.1 CH₂ | 101.1 CH₂ | 102.1 CH₂ |
| 3′,4′ | | | |
| AcO-4″ | 170.1 C | | |
| | 21.1 CH₃ | | |

^{a}Assignments of chemical shifts are based on the analysis of 1D- and 2D-NMR spectra. CH₃, CH₂, CH, and C multiplicities were determined by DEPT 90, DEPT 135, and HSQC experiments.
^{b}Data (δ) measured at 150.9 MHz in DMSO-d₆ and referenced to residual DMSO-d₆ at δ 39.52.
^{c}Data (δ) measured at 100.6 MHz in DMSO-d₆ and referenced to residual DMSO-d₆ at δ 39.52.
^{d}Data (δ) measured at 100.6 MHz in acetone-d₆ and referenced to residual acetone-d₆ at δ 29.84.
^{f}Exist in pairs.

Phyllanthusmin A (6): Amorphous colorless powder showing a blue color under UV light at 365 nm; UV (MeOH) λₘₐₓ (log ε) 262 (4.61) nm; IR (dried film) vₘₐₓ 3447, 1766, 1716, 1597, 1508, 1480, 752 cm⁻¹; positive-ion HRESIMS m/z 40:3.0794, calcd for C₂₁H₁₆O₇Na, 403.0794.

7-O-((2,3,4tri-O-acetyl)-α-L-arabinopyranosyl) diphyliin (7): Amorphous colorless powder showing a blue color under UV light at 365 nm; [α]²⁰_{D} −12.0 (c 0.05, CHCl₃); UV (MeOH) λₘₐₓ (log ε) 260 (4.45) nm; CD (MeOH, nm) λₘₐₓ (Δε) 295 (−3.87); IR (dried film) vₘₐₓ 1749, 1619, 1506, 1480, 770 cm⁻¹; positive-ion HRESIMS m/z 661.1553, calcd for C₃₂H₃₀O₁₄Na, 661.1533.

Diphyllin (8): Amorphous colorless powder showing a blue color under UV light at 365 nm: UV (MeOH) λₘₐₓ (log ε) 267 (4.59) run; IR (dried film) vₘₐₓ 1705, 1615, 1506, 1489, 774 cm⁻¹; positive-ion HRESIMS m/z 403.0797, caled for C₂₁H₁₆O₇Na, 403.0794.

TABLE 4
¹H NMR Spectroscopic Data of Compounds 6-8^{a}.

| position | 6^{b} | 7^{b} | 8^{c} |
|---|---|---|---|
| 3 | 7.21 s | 7.07 s | 7.09 s |
| 6 | 7.56 s | 7.54 s | 7.70 s |
| 9 | 5.55 br s | 5.50 d (15.6) | 5.37 s |
| | | 5.44 dd (14.8, 1.6) | |
| 2′ | 6.78 overlapped | 6.82 overlapped | 6.85 d (1.2) |
| 5′ | 6.95 d (7.6) | 6.97 d (8.0) | 6.97 d (8.0) |
| 6′ | 6.78 overlapped | 6.82 overlapped | 6.82 dd (8.0, 1.6) |
| 1″ | | 5.10 d (7.2) | |
| 2″ | | 5.72 dd (9.6, 7.2) | |
| 3″ | | 5.19 dd (9.6, 3.6) | |
| 4″ | | 5.38 br s | |
| 5″ₐₓ | | 3.73 br d (12.8) | |
| 5″_{eq} | | 4.21 dd (13.2, 2.8) | |
| MeO-4 | | 3.81 s | 3.73 s |
| MeO-5 | 4.10 s | 4.09 s | 4.00 s |
| MeO-7 | 4.13 s | | |
| OCH₂O-3′,4′ | 6.05 s | 6.05 s | 6.08 s |
| | 6.06 s | 6.10 s | 6.09 s |
| AcO-2″ | | ^{d}2.08 s | |
| AcO-3″ | | ^{d}2.12 s | |
| AcO-4″ | | ^{d}2.22 s | |
| HO-4 | 5.96 s | | |

^{a}Chemical shifts were assigned based on the analysis of 1D- and 2D-NMR spectra. The overlapped signals were assigned from ¹H-¹H COSY, HSQC, and HMBC spectra without designating multiplicity (s = singlet, br s = broad singlet, d = doublet, br d = broad doublet, dd = double doublet, m = multiplet). Proton coupling constant J (in parentheses) values are presented in Hz and were omitted if the signals overlapped as multiplets.
^{b}Data (δ) recorded at 400.1 MHz in CDCl₃ and referenced to residual CDCl₃ at δ 7.26.
^{c}Data (δ) recorded at 400.1 MHz in acetone-d₆ and referenced to residual acetone-d₆ at δ 2.05.
^{d}Interchangeable signals.

TABLE 5
¹³C NMR Spectroscopic Data of Compounds 6-8^{a}.

| position | 6^{b} | 7^{b} | 8^{c} |
|---|---|---|---|
| 1 | 125.8 C | 126.3 C | 124.5 C |
| 2 | 131.4 C | 130.9 C | 131.1 C |
| 3 | 109.9 CH | 106.4 CH | 106.8 CH |
| 4 | 146.8 C | 150.5 C | 151.3 C |
| 5 | 149.4 C | 152.1 C | 152.1 C |
| 6 | 100.3 CH | 100.6 CH | 101.3 CH |
| 7 | 148.0 C | 144.3 C | 145.7 C |
| 8 | 123.9 C | 127.4 C | 122.8 C |
| 9 | 66.8 CH₂ | 67.0 CH₂ | 67.0 CH₂ |
| 1′ | 128.5 C | 128.3 C | 130.2 C |
| 2′ | 111.0 CH | 110.8 CH | 112.0 CH |
| 3′ | 147.5 C | 147.7 C | 148.3 C |
| 4′ | 147.5 C | 147.7 C | 148.0 C |
| 5′ | 108.3 CH | 108.4 CH | 108.6 CH |
| 6′ | 123.8 CH | 123.69/123.67 | 124.8 CH |
| | | CH^{e} | |
| 7′ | 134.8 C | 136.4 C | 131.6 C |
| 8′ | 119.6 C | 119.4 C | 120.0 C |
| 9′ | 169.7 C | 169.6 C | 170.3 C |
| 1″ | | 101.6 CH | |
| 2″ | | 69.5 CH | |
| 3″ | | 70.3 CH | |
| 4″ | | 67.39/67.01 | |
| | | CH^{e} | |
| 5″ | | 64.1 CH₂ | |
| MeO-4 | | 56.0 CH₃ | 55.7 CH₃ |
| MeO-5 | 56.4 CH₃ | 56.4 CH₃ | 56.1 CH₃ |
| MeO-7 | 59.8 CH₃ | | |
| OCH₂O-3′,4′ | 101.3 CH₂ | 101.4 CH₂ | 102.1 CH₂ |
| AcO-2″ | | ^{f}170.4 C | |
| | | ^{g}20.9 CH₃ | |
| AcO-3″ | | ^{f}170.3 C | |
| | | ^{g}21.1 CH₃ | |
| AcO-4″ | | ^{f}169.6 C | |
| | | ^{g}21.1 CH₃ | |

^{a}Assignments of chemical shifts are based on the analysis of 1D- and 2D-NMR spectra. CH₃, CH₂, CH, and C multiplicities were determined by DEPT 90, DEPT 135, and HSQC experiments.
^{b}Data (δ) measured at 100.6 MHz in CDCl₃ and referenced to residual CDCl₃ at δ 77.16.
^{c}Data (δ) measured at 100.6 MHz in acetone-d₆ and referenced to residual acetone-d₆ at δ 29.84.
^{e}Exist in pairs.
^{f}Interchangeable signals.
^{g}Interchangeable signals.

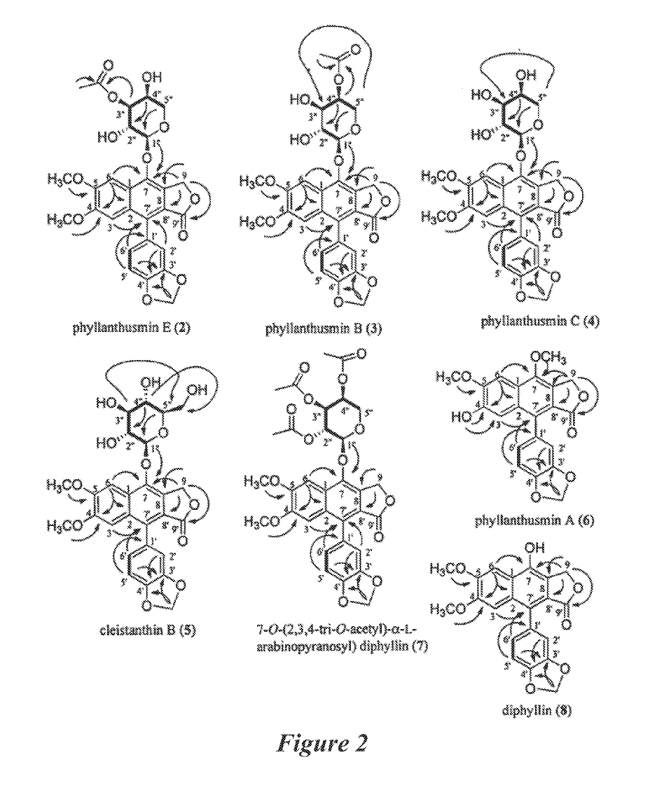

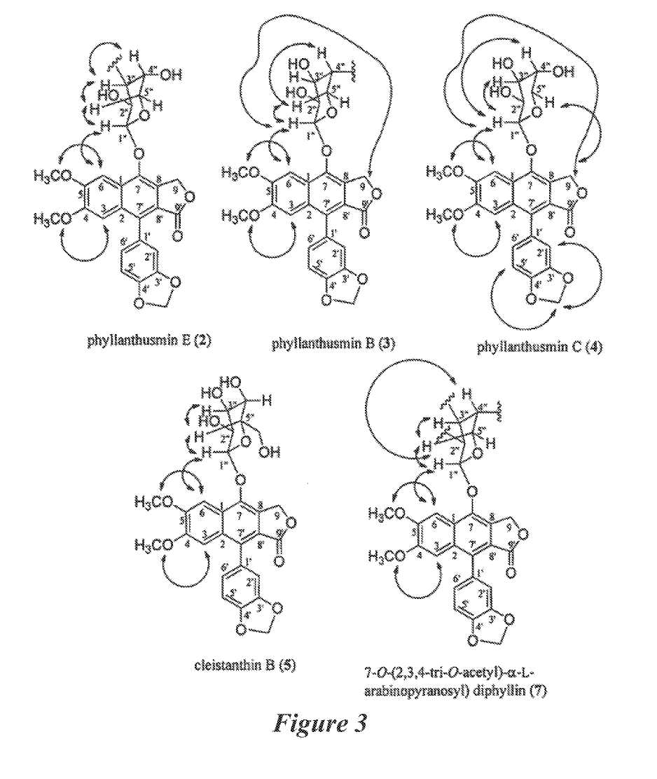

The COSY and key HMBC correlations of compounds 2-8 are shown in FIG. 2. Selected NOESY correlations of compounds 2-5 and 7 are shown in FIG. 3.

X-ray Crystal Structure Analysis of Phyllanthusmin D (1). Intensity data for a small colorless needle (mp 210-211° C.; molecular formula C₃₀H₂₈O₁₃, MW=596.52, hexagonal, space group P6₁22, a=21.4292(6) Å, c=21.4162(6) Å, V=8517.0(4) Å³, Z=12, density (calculated)=1.396 mg/m³, size 0.01×0.01×0.20 mm³) from 1. were collected at 150K on a D8 goniostat equipped with a Bruker APEXII CCD detector at Beamline 11.3.1 using synchrotron radiation tuned to λ=1.2399 Å at the Advanced Light Source at Lawrence Berkeley National Laboratory. For data collection, frames were measured for duration of 1 sec for low angle data and 4 sec for high angle data at 0.3° intervals of co with a maximum 20 value of around 91°. The data frames were collected using the program APEX2 and processed using the program SAINT within APEX2 (APEX2 v2010.3.0 and SAINT v7.60A data collection and data processing programs, respectively). The data were corrected for absorption and beam corrections based on the multi-scan technique as implemented in SADABS (Bruker Analytical X-ray Instruments, Inc., Madison, Wis.; SADABS v2008/1 semi-empirical absorption and beam correction program; G. M. Sheldrick, University of Gottingen, Germany).

The structure was solved by direct methods in SIR-2004 (Burla M C et al. J Appl. Cryst. 2005, 38, 381-388). Full-matrix least-squares refinements based on F² were performed in SHELXL-97 (Sheldrick G M. Acta Cryst. 2008, A64, 112-122), as incorporated in the WinGX package (Farrugia L J. J Appl. Cryst. 1999, 32, 837-838). The benzodioxole group of this molecule is disordered over two sites. During refinement it was necessary to apply distance restraints for this group along with restraints on the anisotropic displacement parameters (SIMU and DELU). For each methyl group, the hydrogen atoms were added at calculated positions using a riding model with U(H)=1.5*Ueq (bonded carbon atom). The torsion angle, which defines the orientation of the methyl group about the C—C or O—C bond, was refined. The hydroxy group hydrogen atom bonded to O (8) was refined isotropically and is involved in an intermolecular hydrogen bond with atom O (2). The rest of the hydrogen atoms were included in the model at calculated positions using a riding model with U(H)=1.2*Ueq (bonded atom). The final refinement cycle was based on 4507 intensities, 191 restraints and 478 variables and resulted in agreement factors of R1(F)=0.050 and wR2(F₂)=0.089. For the subset of data with I>2*sigma(I), the R1(F) value is 0.038 for 3850 reflections. The final difference electron density map contains maximum and minimum peak heights of 0.12 and −0.16 e/Å³. Neutral atom scattering factors were used and include terms for anomalous dispersion. The CIF file of the X-ray data of 1 has been deposited in the Cambridge Crystallographic Data Centre (deposition no.: CCDC 981532).

Acetylation of Phyllanthusmins C (4) and D (1) to 7-O-((2,3,4-Tri-O-acetyl)-α-L-arabinopyranosyl) diphyllin (7). To a dried 25 rnL flask equipped with water condenser and magnetic stirrer, containing 3.0 mg of phyllanthusmin D (I), 5 μL of acetic anhydride and 1 mL pyTidine were added. After the mixture was stirred at 40° C. for 1 h, it was cooled to room temperature. Then, 5 mL of CHCl₃ were transferred into the flask, and the solution was extracted with distilled 1-120. The organic layer was washed with distilled H₂O, and then evaporated at reduced pressure. The residue was purified by silica gel column chromatography, using n-hexane-acetone (5:11:1), to afford 7-O-((2,3,4-tri-O-acetyl)-α-L-arabinopyranosyl) diphyllin {7(1.0 mg, [α]²⁰_{D} −12.0 (c 0.05 CHCl₃)}. Using the same protocol, 5.0 mg of phyllanthusmin C (4) were reacted with 10 ₗaL of acetic anhydride and 2 mL pyridine at 60° C. for 1 h and yielded 1.0 mg of 7 {[α]²⁰_{D} −12.0 (c 0.05, CHCl3)}. These values are very close to that of {[α]²⁰_{D} −13 (c 0.3, CHCl3)} reported for synthetic 7 (Zhao Y et al. Arch. Pharm. Chem. Life Sci. 2012, 345, 622-628).

Acid Hydrolysis of Phyllanthusmin C (4) to Diphyllin (8). To a dried 25 mL flask equipped with water condenser and magnetic stirrer containing phyllanthusmin C (4, 5.0 mg dissolved in 1 mL of MeOH), 5 mL of 37% hydrochloric acid (HCl) were transferred into the flask. After the mixture was stirred at 70° C. for 30 min, the mixture was cooled to room temperature and diluted by 0.1 N NaOH to pH 7.0. Then, 5 mL of CHCl₃ were transferred into the flask, and the solution was extracted with distilled H₂O. The organic layer was washed with distilled H₂O, and then evaporated under reduced pressure. The residue was separated by silica gel column chromatography, using n-hexane and acetone (3:1), to afford diphyllin (8, 1.5 mg).

Cytotoxicity against HT-29 Cells. The cytotoxicity of the test compounds was screened against HT-29 cells by a previously reported procedure (Ren Y et al. J. Nat. Prod 2011, 74, 1117-1125). Paclitaxel and etoposide were used as positive controls.

Cytotoxicity against CCD-112CoN Cells. Following a previous procedure (Still P C et al. J. Nat. Prod. 2013, 76, 243-249) and the method for screening cytotoxicity towards HT-29 cells mentioned above, the cytotoxicity of the samples was screened against CCD-112CoN normal human colon cells.

In Vivo Hollow Fiber Assay. The hollow fiber assay is an excellent method for evaluating the potential of natural products for activity in vivo. The human colon cancer cell line HT-29 was used to evaluate 1 using procedures previously described (Mi Q et al. J. Nat. Prod 2009, 72,573-580; Pearce C J et al. Methods Mol. Biol, 2012, 944, 267-277). Eight- to nine-week-old immunodeficient NCr nu/nu mice were purchased from The Jackson Laboratory (Bar Harbor, Me., USA) and housed in microisolation cages at room temperature and a relative humidity of 50-60% under 12:12 h light-dark cycle. All animal work was approved by University of Illinois at Chicago Animal Care and Use Committee, and the mice were treated in accordance with the institutional guidelines for animal care. Phyllanthusmin D (1) was dissolved initially in DMSO and subsequently diluted with CREMOPHOR™. The mixture was diluted with distilled water to 13% DMSO and 25% CREMOPHOR™. The mice were injected ip once daily for four days with 1 or the positive control (paclitaxel). Each mouse was weighed daily during the study. Animals showed no signs of toxicity even at the highest concentration of 1, and all the remaining mice were sacrificed on day 7. The fibers were retrieved and viable cell mass was evaluated by a modified MTT (3-(4,5-dimethylthiazol-2-yl)-2,5-diphenyltetrazolium bromide) assay. The percentage of the net growth for the cells in each treatment group was calculated by subtracting the day 0 absorbance from the day 7 absorbance and dividing this difference by the net growth in the vehicle control (minus value between the day 7 and the day). Data were compared by the Student's t test, and a p value less than 0.05 was considered statistically significant.

Topoisomerase II Assay. Topo II-DNA covalent complexes induced by topo II poisons such as etoposide may be trapped by rapidly denaturing the complexed enzyme with sodium dodecyl sulfate (SDS), digesting away the enzyme, and releasing the cleaved DNA as linear DNA. The formation of linear DNA was detected by separating the SDS-treated reaction products using ethidium bromide gel electrophoresis by a modification of a previously described procedure (Hasinoff B B et al. Mol. Pharmacol. 2005, 67, 937-947). In this system, topo II-mediated catalytic conversion of supercoiled pBR322 DNA to the “relaxed” form of plasmic' DNA can also be observed. A 20 μL cleavage assay reaction mixture contained 250 ng topo IIα protein, 160 ng pBR322 plasmid DNA (NEB, Ipswich, Mass.), 1.0 mM ATP in assay buffer (10 mM Tris-HCl (pH 7.5), 50 mM KCl, 50 mM NaCl, 0.1 mM EDTA, 5 mM MgCl₂, 2.5% glycerol), and 100 μM of test compounds or DMSO solvent, as indicated. Assay buffer (17 μL) and test compound/DMSO (1 μL) were mixed and allowed to sit at room temperature for 30 min after which 2 μL of topo IIα was added to initiate the reaction. Tubes were incubated at 37° C. for 15 min, and then quenched with 1% (v/v) SDS/10 mM disodium EDTA/200 mM NaCl. The mixture was treated subsequently with 0.77 mg/mL proteinase K (Sigma) at 55° C. for 60 min to digest the protein, and DNA bands were separated by electrophoresis (18 h at 2 V/cm) on an agarose gel (1.3% w/v) containing 0.7 μg/mL ethidium bromide in TAE buffer pH 8.0 (40 mM Tris base, 0.114% (v/v) glacial acetic acid, 2 mM EDTA). The DNA in the gel was imaged by its fluorescence on a Chemi-Doc XRS+ imager (Bio-Rad, Hercules, Calif.). Linear DNA was quantified by accounting for the relationship between fluorescence and relative band intensity for open circular (QC), linear (LNR), supercoiled (SC), and RLX (relaxed) configurations of DNA (Projan S J et al. Plasmid 1983, 9, 182-190), then calculating % of LNR from the total DNA content in each lane. Results are shown for etoposide, phyllanthusmins C (4) and D (1), and 7-O-((2,3,4-tri-O-acetyl)-α-L-arabinopyranosyl) diphyllin (7) in replicate experiments performed on separate days.

Annexin V Staining Method. As described in previous studies (Ren Y et al. ACS Med. Chem. Lett. 2012, 3, 631-636), HT-29 cells were treated with the vehicle control, etoposide (1 or 5 μM), or 1 (1 or 5 μM) for 72 h. The cells were washed with Annexin V binding buffer, centrifuged at 300×g for 10 min, and suspended (1×10⁶) in 100 μL, of 1× Annexin V binding buffer. Then, 10 μL of Annexin fluorochrome was added to the suspension. After the suspension was mixed and incubated in a dark room at room temperature for 15 min, the cells were centrifuged, and the cell pellet was resuspended (1×10⁶) in 100 μL of 1× Annexin V binding buffer. Next, 10 μL of Anti-Biotin-APC were added, and the cells incubated at 4-8° C. in a dark room for 10 min were centrifuged, The cell pellet was resuspended (1×10⁶) in 500 μL of 1× Annexin V Binding Buffer. After 5 μg/mL of 7-AAD solution was added in the suspension, flow cytometry was conducted immediately.

Western Blot Analysis. After a 2.4 h treatment, HT-29 cells were harvested, washed once with ice-cold PBS, and lysed (10⁸ cells/mL lysis buffer) in hypertonic buffer {1% NP-40, 10 mM HEPES (pH 7.5), 0.5 M NaCl, 10% glycerol supplemented with protease and phosphatase inhibitors (1 mM phenylmethylsulfonylfluoride (PMSF), 1 mM Na₃VO₄, 50 mM NaF, 10 mM β-glycerol phosphate, 1 mM EDTA), and protease inhibitor cocktail tablet (Roche Applied Science, Indianapolis, Ind., USA). Cell lysates, adding 4 or 2× laemmli buffer (Bio-Rad) by supplementing with 2.5% β-mercaptoethonat to give 1× SDS sample buffer, were boiled for 5 min and subjected to Western blot analysis, as described previously (Yu J et al, Immunity 2006, 24, 575-590). To determine protein concentrations, samples were first solubilized in NP-40 lysis buffer and protein levels were assessed using a BCA protein assay kit (Bio-Rad), standardized with BSA. Protein samples were resolved on 4-15% SDS-PAGE (Bio-Rad) and immunoblot analysis was performed using Abs against the indicated signaling molecules. The antibodies used were: rabbit monoclonal Cleaved Caspase-3(Asp175) (Cell Signaling Technology, Beverly, Mass.) and goat polyclonal β-actin (Santa Cruz Biotechnology, Santa Cruz, Calif.).

### Results and Discussion

When the cytotoxic chloroform partitions were subjected to chromatographic separation guided by inhibitory activity against the HT-29 cell line, several arylnaphthalene lignan lactones (phyllanthusmins A (6), B (3), C (4), D (1) and E (2) and cleistanthin B (5)) (Wu S J and Wu T S. Chem, Pharm, Bull. 2006, 54, 1223-1225; Al-Abed Y et al. J. Nat. Prod 1990, 53, 1152-1161) were purified.

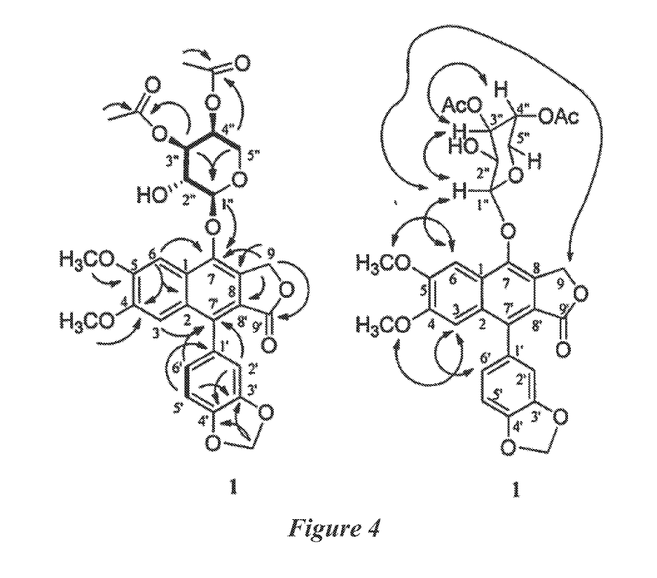

Compound 1 was isolated in the form of colorless fine needles (mp 210-211° C.). A sodiated molecular ion peak at nilz 619.1444 (calcd 619.1428) observed in the HRESIMS corresponded to a molecular formula of C₃₀H₂₈O₁₃. The UV (λₘₐₓ 260 nm) and IR (vₘₐₓ 3446 (hydroxy), 1747 (γ-lactone), 1619, 1507, and 1481 (aromatic) cm⁻¹) spectra showed the absorption characteristics of an arylnaplithalene lignan lactone (Rezanka T et al. Phytochemistry 2009, 70, 1049-1054), The ¹H NMR spectrum of 1 (Table 1) (Gottlieb H E et al. J. Org. Chem. 1997, 62, 7512-7515) exhibited signals for two substituted aromatic rings at δ_{H} 6.81, 6.83, 6.97, 7,09, and 7.94, a lactone methylene group at OH 5.47 and 5.56, a methylenedioxy group at δ_{H} 6.05 and 6.10, two methoxy groups at δ_{H} 3.81 and 4.03, two acetyl groups at δ_{H} 2.14 and 2.23, and proton signals for a sugar moiety in the range δ_{H} 3.60-4.99 (Tuchinda P et al. Planta Med. 2006, 72,60-62). Analogous signals consistent with the presence of these functionalities appeared in the ¹³C NMR spectrum of 1 (Table 1) (Abdullaev N D et al. Khim. Prir. Soedin. 1987, 76-90). The lactone ring was proposed to occur at the C-8 and C-8′ positions, as supported by the HMBC correlations between H-9/C-8, C-8′, and C-9′ (FIG. 4), The methylenedioxy group could be located at the C-3′ and C-4′ positions, as indicated by the HMBC correlations between these methylene protons and C-3′ and C-4′. Two methoxy groups were assigned at the C-4 and C-5 positions from the HMBC correlations between these methoxy groups and C-4 and C-5. The sugar unit was assigned to the C-7 position, as supported by the HMBC correlation between H-1″ and C-7. Two acetyl groups were placed at the C-3″ and C-4″ positions of the sugar residue, as indicated by the HMBC correlations between the H-3″ and H-4″ signals and the acetyl carbonyl groups. The resonances corresponding to the signals at δ 3.52 for H-3″ and at δ 3.71 for H-4″ appeared in the ¹H NMR spectrum of 4 (Table 2) were shifted downfield to the signals at δ 4.99 (H-3″) and d 5.30 (H-4″) in the ¹H NMR spectrum of 1, due to the electron-withdrawing effects that resulted from the acetyl carbonyl groups linked at the C-3″ and C-4″ positions, This was also supported by the molecular weight of 596 Da of 1, or 42 atomic mass units more than that of 3, representing the presence of a diacetylglycose residue in 1 rather than a monoacetylglycose unit in 3. Thus, compound 1 was proposed as an acetyl analogue of the compounds phyllanthusmin B (3) and phyllanthusmin C (4), with both being characterized from Phyllanthus oligospermus in a previous study (Wu S J and Wu T S. Chem. Pharm. Bull. 2006, 54, 1221-1225).

Comparison of the NMR data of compound 1 with those of phyllanthusmins B (3) and C (4) (Table 1, Table 2 and Table 3) showed that these compounds displayed closely similar NMR signals for the diphyllin aglycone unit but different resonances for their sacch.aride portions. An L-arabinose residue of 1 could be proposed based on several lines of evidence. First, both the NOESY correlations and the optical rotation value of 1 were consistent with those of compound, 3, as reported in the literature (Wu S J and Wu T S. Chem. Pharm. Bull. 2006, 54, 1223-1225) and isolated in the present study. Second, the NOESY correlations and optical rotation value of 1 were consistent with those determined for 4 (phyllanthusmin C) in this investigation. The latter compound, when isolated from P. poilanei, showed a closely comparable optical rotation value {[a]²⁰_{D}−8.0 (c 0.06, CHCl₃)} to that obtained when synthesized from diphyllin and L-arabinose [−7.5 (c 1.04, CHCl₃)] (Shi D K et al. Eur. J. Med. Chem. 2012, 47, 424-431). Finally, both 1 and 4 were acetylated to form the same compound, 7-O-((2,3,4-tri-O-acetyl)-α-L-arabinopyranosyl) diphyllin (7), which exhibited the same optical rotation value of [α]²⁰_{D} −12.0 (c 0.05, CHCl₃) for both products, and was almost identical with that of a synthetic version of compound 7, [α]²⁰_{D} −13 (c 0.3, CHC13), as reported in the literature (Zhao Y et al. Arch. Pharm. (hem. 14,le 2012, 345, 622-628).

The doublet due to H-1″ at δ_{H} 4.86 with a coupling constant of 7.60 Hz indicated the presence in 1 of an anomeric proton in an axial orientation (Wu S J and Wu T S. Chem. Pharm. Bull. 2006, 54, 1223-1225; Fischer M H et al. Carbohydr. Res. 2004, 339, 2009-2017). The NOESY correlations between H-1″ and H-3″ and H-3″ and H-4″ suggested that H-1″, H-3″, and AcO-4″ are all axial (FIG. 4). Thus, the structure of this compound (phyllanthusmin D) was assigned as 7-O-(3,4-di-O-acetyl)-α-L-arabinopyranosyl-4,5-ditnethoxy-3′,4′-methylenedioxy-2,7′-cycioligna-7,7′-dieno-9,9′-lactone, or 7-O-[(3,4-di-O-acetyl)-α-L-arabinopyranosyl] diphyllin.

The structure of 1 was confirmed by analysis of its single-crystal X-ray diffraction data. This compound existed in two conformational forms because of a hindered rotation in ring D, which resulted in the ¹³C NMR signal of C-8 being split into two signals at δ 131.42 and 131.43 (Table 1). The same hindered rotation would likely be observed in 3-5 and other arylnaphthalene lignan lactones, as indicated by the split signals that appeared in their NMR spectra (Table 3) (Tuchinda P et al. Planta Med. 2006, 72, 60-62; Tuchinda P et al. J. Nat. Prod 2008, 71, 655-663). All these compounds exist as atropisomers, most of which interconvert slowly at room temperature, as described in a previous dynamic NMR study, which showed that the hindered rotation allowed arylnaphthalene lignans to exist as two diastereomers long enough to be observed in the NMR spectra, but the rotation barrier was too small for the individual diastereomers to be isolated at room temperature (Charlton J L et al. J. Org. Chem. 1996, 61, 3452-3457).

Compound 2 was isolated in the form of an amorphous colorless powder. The similar UV and IR spectra with those of 1 indicated that 2 is also an arylnaphthalene lignan. The molecular formula of C₂₈H₂₆O₁₂ deduced from a sodiated molecular ion peak at m/z 577.1319 (calcd 577.1322) observed in the HRESIMS and the similar NMR data with those of 1 indicated that this compound is a diphyllin monoacetylarabinoside and a close analogue of phyllanthusmin B (3) (Wu S J and Wu T S. Chem. Pharm. Bull. 2006, 54, 1223-1225). Comparison of the ¹H and ¹³C NMR data with those of 3 showed that the signals for H-3″ and C-3″ of 2 were shifted downfield, but the signals for its H-4″ and C-4″ were shifted upfield (Table 1, Table 2, and Table 3). This indicated that the acetyl group is attached to the C-3″ position in 2 rather than to the C-4″ position in 3, as supported by the HMBC correlation between the H-3″ and the acetyl carbonyl group signal. A doublet at δ_{H} 4.84 showing a coupling constant of 7.34 Hz displayed in the ¹H NMR spectrum of 2 supported the presence of the anomeric proton in an axial orientation (Wu S J and Wu T S. Chem. Pharm. Bull. 2006, 54, 1223-1225; Fischer M H et al. Carhohydr. Res. 2004, 339, 2009-2017). The NOESY correlations between H-1″ and H-5″ₐₓ and H-3″ and H-4″ and H-5″ₐₓ suggested that H-1″, H-3″, and OH-4″ are all axial (FIG. 3).

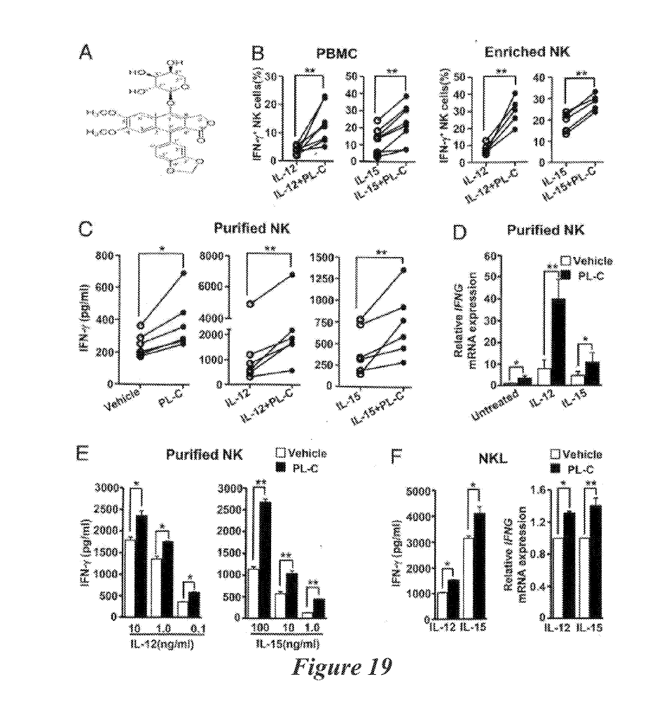

Although 2 was not hydrolyzed to yield the sugar unit or to acetylated into 7-O-((2,3,4-tri-O-acetyl)-α-L-arabinopyranosyt) diphyllin (7), the consistent NOESY correlations (FIG. 19) and optical rotation values measured {[α]²⁰_{D}−3.3 (c 0.09, CHCl3) for 1, [α]²⁰_{D} −4.4 (c 0.09, CHCl3) for 2, [α]²_{D} −6.0 (c 0.05, CHCl3) for 3, and [α]²⁰_{D} −8.0 (c 0.06, CHCl3) for 4} suggests that compound 2 can have the same absolute configuration as that of 1, 3, and 4. Therefore, the structure of 2 (phyllanthusmin E) could be proposed as 7-O-(3-O-acetyl)-α-L-arabinopyranosyl-4,5-dimethoxy-3′,4′-methylenedioxy-2,7′-cycloligna-7,7′-dieno-9,9′-lactone, or 7-O-[(3-O-acetyl)-α-L-arabinopyranosyl] diphyllin.

The other arylnaphthalene lignan lactones isolated from P. poilanei were identified by analysis of their spectroscopic data and comparison with literature values (Wu S J and Wu T S. Chem. Pharm. Bull. 2006, 54, 1223-1225; Al-Abed Y et al, J. Nat. Prod 1990, 53, 1152-1161; Abdullaev N D et al. Khim Prir. Soedin. 1987, 76-90), and full assignments of their ¹H and ¹³C NMR spectroscopic data are listed in Tables 2-5. To partially discern the effects of both the arabinose unit and the acetyl group in the mediation of cytotoxicity of the diphyllin lignans obtained in the present study, two additional analogues were prepared. 7-O-[(2,3,4-Tri-O-acetyl)-α-L-arabinopyranosyl] diphyllin (7) was produced by acetylation of 1 and 4 using a standard method (Ren Y et al. J. Nat. Prod 2011, 74, 1117-1125), and was identified by its molecular formula of C₃₂H₃₀O₁₄, as determined by HREIMS and comparison of its spectroscopic data with those reported for a synthesized form of this compound (Zhao Y et al. Arch. Pharm. Chem. Life Sci. 2012, 345, 622-628). The aglycone, diphyllin (8), was generated by hydrolysis of phyllanthusmin C (4) and determined by comparison of its spectroscopic data with reference values (Abdullaev N D et al. Khim. Prir. Soedin. 1987, 76-90; Okigawa M et al. Tetrahedron 1910, 26, 4301-4305).

All arylnaphthalene lignans isolated from P. poilanet in the present study and their semi-synthetic analogues were evaluated for their cytotoxicity against HT-29 human colon cancer cells, using paclitaxel as the positive control (Table 6) (Ren Y et al. J. Nat. Prod 2011, 74, 1117-1125). Compounds 1-4, 7 and 8 were found to be cytotoxic, of which 1 and 7 were the most potently active, with IC₅₀ values of 170 and 110 nM, respectively.

Inspection of the lignan structures and their cytotoxicity showed that compounds containing more acetyl groups can exhibit higher potencies, so the presence of one or more acetyl groups linked to the arabinose residue can improve the resultant cytotoxicity. Compounds 2 and 3 exhibited the same activity, indicating that the acetyl group linked to the C-2″ position contributed to this activity equally when linked to C-3″ position. Phyllanthusmin C (4) showed a higher cytotoxicity than diphyllin (8) and cleistanthin B (5), implying that the α-L-arabinose unit at the C-7 position played a role in mediation of this effect and was more active than a β-D-glucose unit in mediating compound cytotoxicity toward HT-29 cells. Diphyllin (8) was active, but phyllanthusmin A (6) was inactive, showing that the methoxy groups at the C-4 and C-5 positions and the hydroxy group linked at the C-7 position all can play a role in the cytotoxic activity of diphyllin.

TABLE 6
Cytotoxicity toward HT-29 and CCD-112CoN Cells of compounds 1-8^{a}.

| compound | HT-29^{b} | CCD-112CoN^{c} |
|---|---|---|
| 1 | 0.17 | >100 |
| 2 | 1.8 | NT |
| 3 | 1.8 | NT |
| 4 | 3.2 | >100 |
| 5 | >10^{d} | NT |
| 6 | >10 | NT |
| 7 | 0.11 | NT |
| 8 | 7.6 | NT |
| paclitaxel^{e} | 0.001 | 23.0 |
| etoposide^{e} | >10 | NT |

^{a}IC₅₀ values were calculated using nonlinear regression analysis with measurements performed in triplicate and representative of two independent experiments in which the values general agreed within 10%.
^{b}Represented as IC₅₀ values (μM) toward the HT-29 cells.
^{c}Represented as IC₅₀ values (μM) toward the CCD-112 CoN cells.
^{d}Showing borderline cytotoxicity with an IC₅₀ value of 12.0 μM.
NT = compound not tested.
^{e}Positive control.

Two cytotoxic isolates, 1 and 4, were tested for their cytotoxicity toward the CCD-112CoN human normal colon cells using a previous protocol (Still P C et al. J. Nat. Prod. 2013, 76, 243-249). Both compounds were found to be non-cytotoxic toward this cell line (Table 6), indicating some selectivity of these compounds for HT-29 human colon cancer cells.

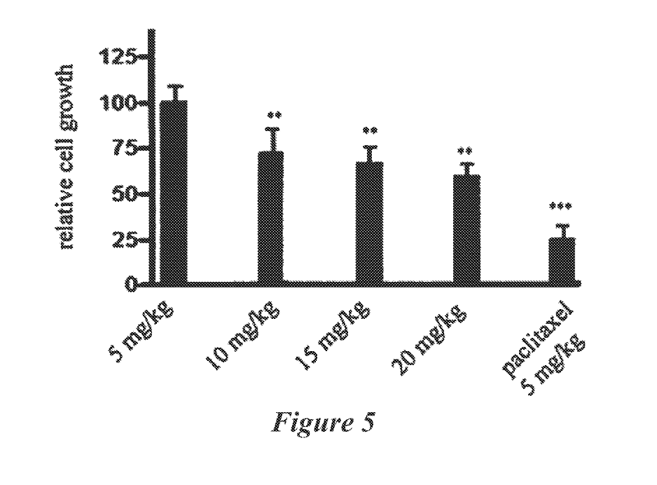

The cytotoxic compound, phyllanthusmin D (1, IC₅₀, 170 nM), isolated from P. poilanei in the present study, was tested further in an in vivo hollow fiber assay for its potential antitumor efficacy (Mi Q et al. J. Nat. Prod 2009, 72,573-580; Pearce C J et al. Methods Mol. Biol. 2012, 944, 267-277). Immunodeficient NCr nu/nu mice implanted with human colon cancer HT-29 cells placed in hollow fibers were treated once daily by 1 at doses of 5.0, 10.0, 15.0, or 20.0 mg/kg, or the vehicle control, or paclitaxel (5 mg/kg), by intraperitoneal (ip) injection tier four days. The relative FIT-29 cell growth values from all mice were calculated. The results showed that the values from the treatment of 1 at 10.0, 15,0, or 20.0 mg/kg (ip) were all statistically significantly different with those at a dose of 5 mg/kg (ip), and they showed a dose dependent tendency (FIG. 5). No gross toxicity was observed in the mice treated at the doses employed.

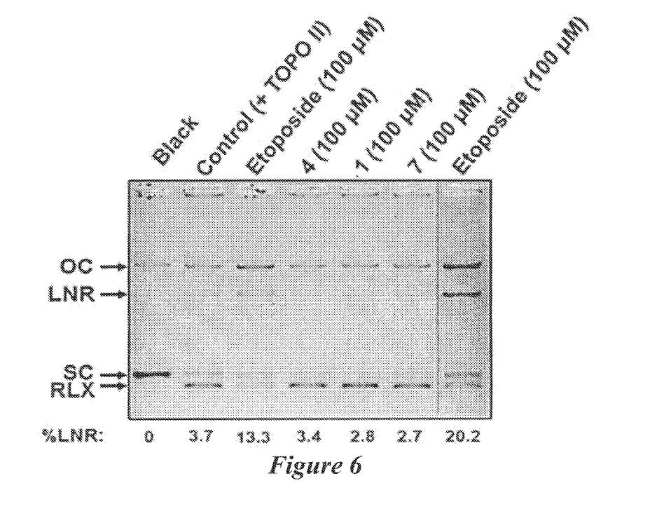

The enzyme DNA topoisomerase (topo II) is an established molecular target of etoposide, on which this compound acts to form DNA double-strand breaks via stabilization of the intermediate topo II-DNA covalent complex to initiate the cell death pathway (Meresse P et al. Curr. Med Chem. 2004, 11, 2443-2466). Several diphyllin arabinosides, including phyllanthusmins C (4) and D (1) and 7-O-((2,3,4-tri-O-acetyl)-α-L-arabinopyranosyl) diphyllin (7), together with etoposide, were tested for their ability to inhibit DNA topo IIα (FIG. 6), using a method reported previously (Hasinoff B B et al. Mol. Pharmacol. 2005, 67, 937-947; Projan S J et al. Plasmid 1983, 9, 182-190). Consistent with a previous report (Meresse P et al. Curr. Med Chem. 2004, 11, 2443-2466), etoposide showed topo Ha inhibitory activity (FIG. 6). However, arylnaphthalene lignan lactones investigated herein neither inhibited topo Ha-mediated DNA strand passage/catalytic activity (conversion of supercoiled DNA (SC) to relaxed DNA (RLX)) nor induced topo Ha-mediated DNA cleavage (linearized double-strand DNA (LNR)) compared to the control, indicating that these arylnaphthalene hpans are not topo Ha inhibitors, Previous reports demonstrated that several diphyllin glycosides inhibited topo 11, but other close analogues of these compounds did not (Zhao Y et al, Arch. Pharm. Chem. Life Sci, 2012, 345, 622-628; Shi DK et al. Eur. J. Med. Chem. 2012, 47, 424-431). This indicates that the glycosidic moiety of these lignans plays a role in topo II inhibition, and some specific diphyllin glycosides might exert their cytotoxicity through a mechanism of action different from that of etoposide (Zhao Y et al. Arch. Pharm. Chem. Life Sci. 2012, 345, 622-628; Shi D K et al. Eur. J. Med. Chem. 2012, 47, 424-431), as supported by additional chemical and biological studies for these types of compounds (Susplugas S et al. J. Nat. Prod. 2005, 68, 734-738; Kang K et al. Neoplasia 2011, 13, 1043-1057).

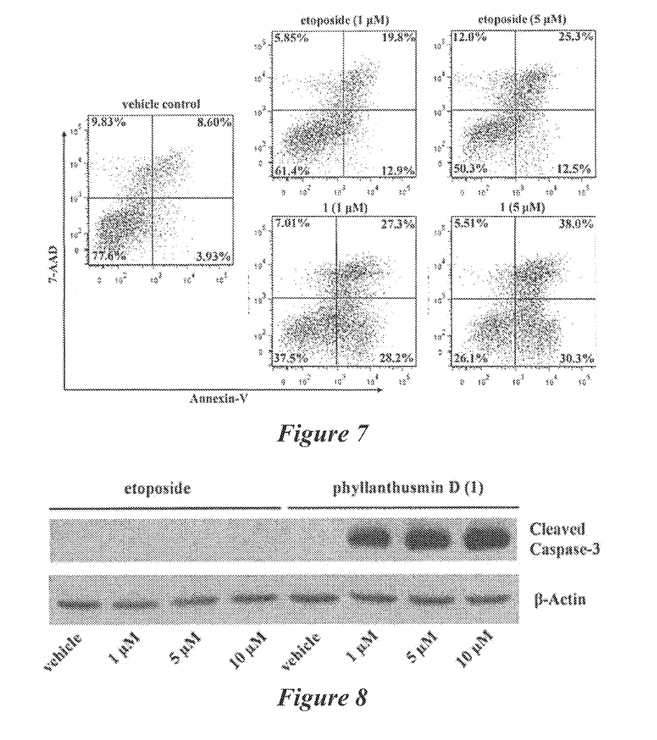

Apoptosis, or programmed cell death, occurs during normal cellular differentiation and the development of multicellular organisms (Joseph B et al. Oncogene 2001, 20, 2877-2888; Woo M et al, Genes Dev. 1998, 12, 806-819). To remain malignant, cancer cells must evade apoptosis to avoid elimination, and many anticancer agents induce cancer cell apoptosis (Woo M et al. Genes Dev. 1998, 12, 806-819). A previous study showed that an eight-day treatment of etoposide induced HT-29 human colon cancer cell apoptosis, but shorter term treatment with this compound did not show this activity (Schonn I et al. Apoptosis 2010, 15, 162-172). After HT-29 cells were treated with compound 1 or etoposide at different concentrations, annexin V flow cytometry was performed following a previous protocol (Ren Y et al. ACS Med. Chem. Lett. 2012, 3, 631-636). Treatment of HT-29 cells with 1 μM or 5 μM phyllanthusmin D (1) for 72 h resulted in 28.2% or 30.3 HT-29 cells undergoing early apoptosis, respectively, while the analogous values for the vehicle control or 1 μM or 5 μM etoposide treatments were 3.9%, 12.9%, and 12.5%, respectively (FIG. 7). Also, 1 induced 27.3% (at 1 μM) and 38.0% (at 5 μM) of HT-29 cell apoptosis at the late-stage, while the vehicle control or 1 μM or 5 μM etoposide treatments induced 8.60% or 19.8%, or 25.3% of HT-29 cell apoptosis at this stage, respectively (FIG. 7). These results indicated that compound I showed a higher potency than etoposide in inducing HT-29 cell apoptosis.

Caspase-3, a key effector of programmed cell death and a well-known anticancer drug target, is only activated during cell apoptosis and contributes fundamentally to this process (Woo M et al. Genes Dev, 1998, 12, 806-819; Li P et al. Cell 2004, S116, S57-S59). Following a previous procedure (Yu J et al. Immunity 2006, 24, 575-590), both 1 and etoposide were tested for their caspase-3 activation in HT-29 cells (FIG. 8). After 24 h incubation, phyllanthusmin D (1) induced a concentration dependent activation of caspase-3. In contrast, under the same experimental conditions, etoposide did not induce caspase-3 activation, which is consistent with the known resistance of HT-29 cells to etoposide (Hwang J T et al. Ann. N. Y. Acad Sci. 2007, 1095, 441-448). These results again indicate a fundamental difference in the mechanism(s) of action of these agents.

### Example 2

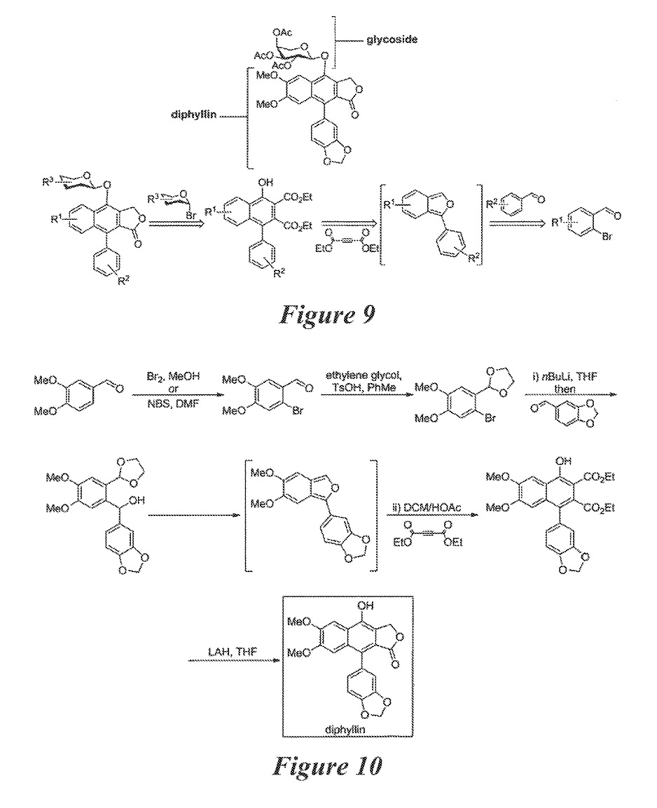

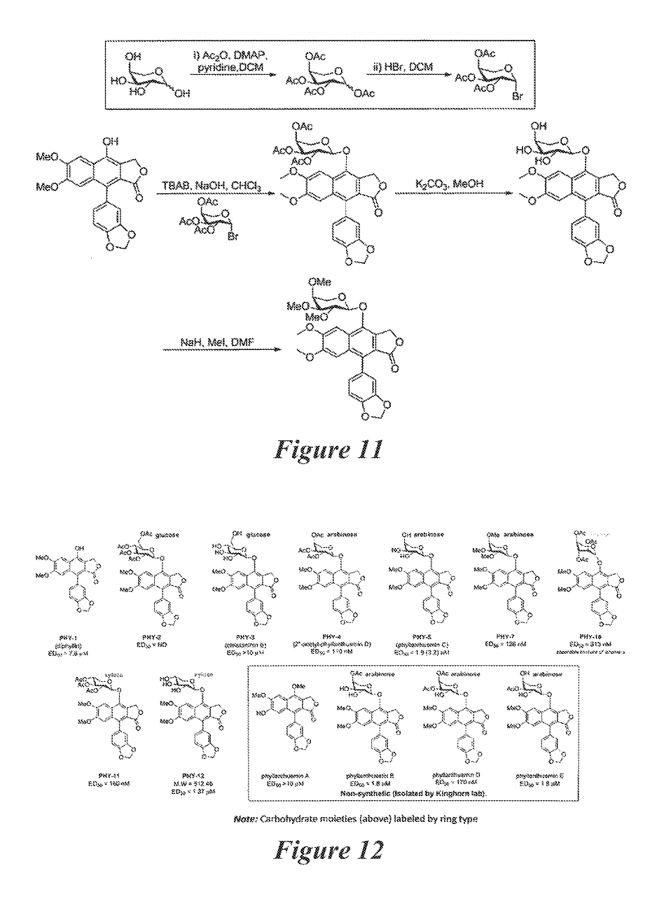

A convergent synthesis of phyllanthusmins through late-stage glycosylation of the diphyllin core was examined (FIG. 9). The diphyllin core was synthesized over three steps (Charlton et al. J. Org. Chem. 1996, 61, 3452-3457) (FIG. 10). This synthesis allowed for rapid individual variation of the napthyl and biaryl ring systems. The diphyllin core underwent phase transfer catalyzed glycosylation via acetylated glycosyl bromide donors (Yu et al. J. Carbohydr. Chem. 2008, 27, 113-119) (FIG. 11). This provided rapid access to phyllanthusmins and other diphyllin glucosides.

The synthesized ₁^{.})11yllanthusm in analogues shown in (FIG. 12) were evaluated in vitro utilizing HT-29 cells.

Phyllanthusmin D and 2″-acetyl-phyilanthusm in I) (natural samples) displayed similar toxicity towards HT-29 cells (ED5o=170 nm and 110 nm, respectively). Phyllanthustnin D was active in an in vivo hollow fiber assay, with no signs of gross toxicity (FIG. 5). Based on this result, 500 mg of 2″-acetyl-phyllanthusmin D, the more synthetically viable target, was prepared for in vivo studies. However, this material was found to be insoluble in aqueous solutions.

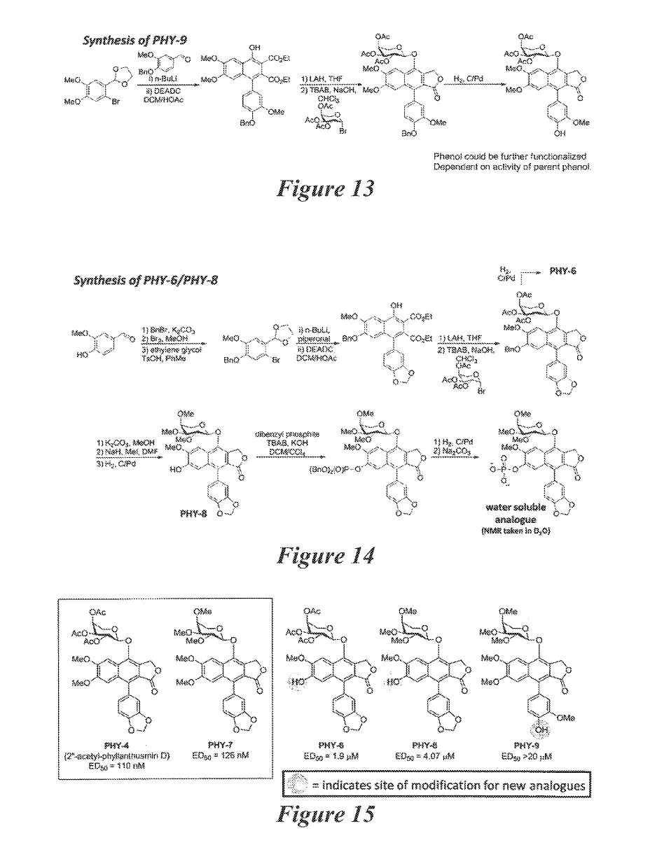

Methods to improve the water solubility of the 2″-acetyl-phyllanthusmin D by incorporating polar substituents into the free alcohol substituents (e.g., phenols) were considered. Accordingly, the diphyllin core containing a protected phenol was synthesized (FIG. 13). The phenol can allow for appending various functional groups to combat the poor water solubility, Cleavage of this group can then reveal the “active” substrate (e.g., a prodrug approach).

Another diphyilin core containing an alternate protected phenol was also synthesized (naphthalene system) (FIG. 14). This compound can allow fOr appending various functional groups to combat poor water solubility, as illustrated by the phosphate derivative in FIG. 14.

The antiproliferative activity of the free phenols was tested in the FIT-29 cell based assay. Losses in activity were observed for all the phyllanthusmin analogues with a free phenol (FIG. 15). The best results were obtained for PHY-6 and PHY-8, which were 10-20 less potent than PHY-4 and PHY-7. As a result, other sites were investigated for introducing water solubilizing functionality.

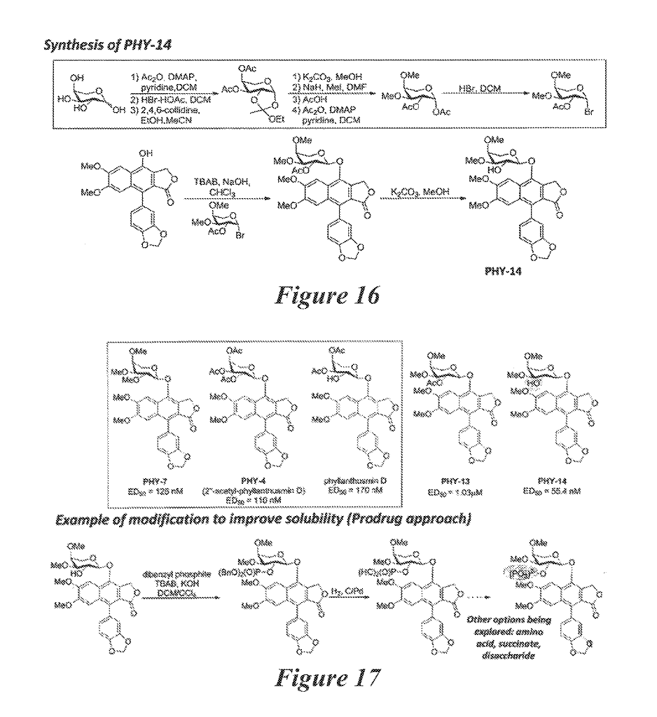

A differentially functionalized arabinose was synthesized (Son et al. Org. Lett. 2007, 9, 3897-3900) (FIG. 16). This can allow for introduction of water solubilizing groups with an intact diphyllin core (FIG. 17).

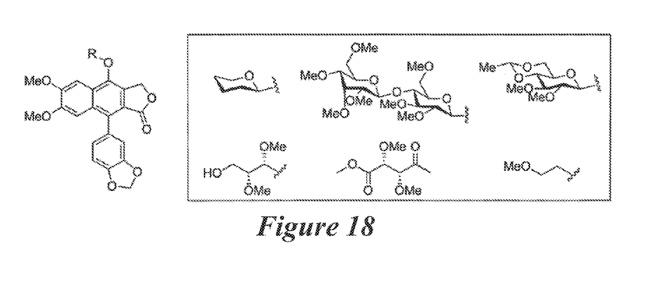

A complete synthesis of a series of analogues focusing on manipulation of the glycoside was discussed herein (FIG. 18). Various solubilizing groups were appended to the synthesized phyllanthusmin D to fine tune the water solubility. Additionally, the mechanism responsible for the biological activity of the phyllanthusmin analogues was investigated.

### Example 3

Etoposide is a semi-synthetic aryltetralin lignin glycoside modeled on the natural product podophynotoxin. It can target DNA topoisomerase II (topo II) and has been used for decades to treat a variety of malignancies. However, side effects have been reported for etoposide, including the development of secondary leukemias linked to topo II inhibitory activity (Ezoe S. Int. J. Environ. Res. Public Health. 2012, 9, 2444-2453). As part of a search for anticancer agents from higher plants and other organisms (Kinghorn A D et al. Pure Appl. Chem. 2009, 81, 1051-1063), several arylnaphthatene lignans (1-8, FIG. 1), close analogues of podophyllotoxin, were Obtained from Phyllanthis poilanei collected in Vietnam. The cytotoxic compound phyllanthusmin D (1, IC₅₀=170 nM against HT-29 cells), showed activity when tested in an in vivo hollow fiber assay without any gross toxicity observed in the mice. Mechanistic studies showed that this compound mediated its cytotoxicity by induction of tumor cell apoptosis through activation of caspase-3 with no inhibitory activity against topo IIα.

All aryinaphthalene lignans obtained from P. poilanei were evaluated for their cytotoxicity against the HT-29 human colon cancer cells, and some of them were tested toward the CCD-112CoN human normal colon cells (Ren Y et al. J. Nat. Prod, 2011, 74, 1117-1125). Some compounds were found to by selectively cytotoxic towards HT-29 cells (Table 6).

Phyllanthusmin D (1, IC₅₀=170 nM toward HT-29 cells) was further tested in an in vivo hollow fiber assay (Mi Q et al. J. Nat. Prod. 2009, 72, 573-580) and was found to be active (FIG. 5).

Compounds 1, 4, and 7 were tested in a topo IIα assay (Hasinoff BB et al. Mol. Pharmacol, 2005, 67, 937-947), Compared to etoposide, all these compounds were not topo IIα inhibitors.

Phyllanthusmin D (1) was tested irr an apoptosis assay (Yu J et al. Immunity. 2006, 24, 575-590). It was found that 1 can induce HT-29 cell apoptosis (FIG. 7).

Phyllanthusmin D (1) was tested via Western Blotting, and it was found to activate caspase-3 (FIG. 8).

Several arylnaphthalene lignans were identified from Phyllanthis poilanei, of which six compounds showed cytotoxicity toward HT-29 human colon cancer cells. Phyilanthusmin D (1) was found to show antitumor efficacy in vivo. Phyilanthusmin D can mediate its cytotoxicity toward HT-29 cells in vitro and in vivo through apoptosis induction involved in caspase-3 activation, rather than topo Ha inhibition.

### Example 4

NK cells are a component of immunity that can destroy cancer cells, cancer-initiating cells, and clear viral infections. However, few reports describe a product that can stimulate NK cell IFN-γ production and unravel a mechanism of action. In this study, through screening, it has been found that phyllanthusmin C (PL-C, 4) alone enhanced IFN-γ production by human NK cells, PL-C also synergized with IL-12, even at the low cytokine concentration of 0.1 ng/mL, and stimulated IFN-γ production in both human CD56^{bright} and CD56^{dim} NK cell subsets. Mechanistically, TLRI and/or TLR6 mediated PL-C's activation of the NF-κB p65 subunit that in turn bound to the proximal promoter of IFNG and subsequently resulted in increased IFN-γ production in NK cells. However, IL-12 and IL-1.5Rs and their related STAT signaling pathways were not responsible for the enhanced IFN-γ secretion by PL-C. PL-C induced little or no T cell IFN-γ production or NK cell cytotoxicity. Collectively, a product has been identified that can enhance human NK cell IFN-γ production.

Natural killer cells (NK cells) are a component of innate immunity, and represent the first line of defense against tumor cells and viral infections (Smyth M J et al. Nat. Rev. Cancer 2002, 2, 850-861). NK cells are large granular lymphocytes with both cytotoxicity and cytokine-producing effector functions, representing a source of IFN-γ in humans (Vivier E et al, Nat. Immunol. 2008, 9, 503-510). IFN-γ has a role in the activation of both innate and adaptive immunity. IFN-γ not only displays antiviral activity (Novelli F and Casanova J L. Cytokine Growth Factor Rev. 2004, 15, 367-377; Lee S H et al. Trends Immunol, 2007, 28, 252-259; Lanier L L. Nat. Rev. Immunol. 2008, 8, 259-268) but IFN-γ can also regulate various cells of the immune system and can perform a role in tumor immunosurveillance (Ikeda H et al. Cytokine Growth Factor Rev. 2002, 13, 95-109) through enhancing tumor immunogenicity and Ag presentation (Kane A and Yang I. Neurosurg. Clin. N. Am, 2010, 21, 77-86) as well as inducing tumor cell apoptosis (Tu S P et al. Cancer Res. 2011, 71, 4247-4259; Hacker S et al. Oncogene 2009, 28, 3097-3110). NK cell-derived IFN-γ can also activate macrophages, promote the adaptive Th1 immune response (Martin-Fontecha A et al. Nat. Immunol. 2004, .5, 1260-1 ₂₆₅)_{,} and regulate CD8⁻ T cell priming (Kos F J and Engleman E G. J. Immunol. 1995, 155, 578-584) and dendritic cell migration during influenza A infection (Kos F J and Engleman E G. J. Immunol, 1995, 155, 578-584; Ge M Q et al. J. Immunol. 2012 189, 2099-2109). In addition, IFN-γ can recruit CD27⁺ mature NK cells to lymph nodes during infection or inflammation (Watt S V et al. J. Immunol. 2008, 181, 5323-5330). Deficiency in NK cell-mediated IFN-γ production can be associated with an increased incidence of both malignancy and infection (Colucci F et al. Nat. Rev. Immunol. 2003, 3, 413-425).

Exogenous recombinant IFN-γ has been used in various cancer immunotherapy trials; however, outcomes have been disappointing because of its toxicity (Dunn G P et al. Nat. Rev. Immunol. 2006,6,836-848). Enhancing endogenous IFN-γ production by stimulation with cytokines such as IL-2, IL-12, IL-15, IL-18, and IL-21, administered either individually or synergistically, has also been tried in preclinical and clinical studies (Wagner K et al. Clin. Cancer Res. 2008, 14, 4951-4960; Jahn T et al. PLoS One 2012, 7, e44482; Strengell M et al. J. Immunol. 2003, 170, 5464-5469; Son Y I et al. Cancer Res. 2001, 61,884-888; Di Carlo E et al. J. Immunol, 2004, 172, 1540-1547). However, these approaches also had limitations (Baer M R et al. J. Clin. Oncol. 2008, 26, 4934-4939), such as induction of regulatory T cells by IL-2 (Gowdy A et al. MAbs 2010, 2, 35-41; Shah M H et al. Clin, Cancer Res, 2006, 12, 3993-3996), impairment of cytokine signaling via STAT-4 as a result of autologous hematopoietic stem cell transplantation or chemotherapy (Robertson M J et al. Blood 2005, 106, 963-970; Chang H C et al. Blood 2009, 113, 5887-5890; Lupov I P et al. Blood 2011, 118, 6097-6106), and the systemic toxicity associated with the exogenous delivery of these cytokines that can, in some instances, activate a multitude of immune effector cells (Salem M L et al. J. Interferon cytokine Res. 2006, 26, 593-608; Amos S M et al. Blood 2011, 118, 499-509).

There are multiple signaling pathways that can affect IFN-γ gene expression and its protein secretion. These include positive signaling pathways, such as the MAPK signaling pathway, the JAK-STAT signaling pathway, the T-BET signaling pathway, and the NT-κB signaling pathway, as well as negative regulation via the TGF-β signaling pathway (Schoenborn J R and Wilson C B. Adv. Immunol. 2007, 96, 41-101). Activation of the MAPK pathway can involve induction of ERK and p38 kinase, in part through the activation of Fos and jun transcription factors (Schoenborn J R and Wilson C B. Adv. Immunol. 2007, 96, 41-101). Binding of IL-12 to its receptor can activate the JAKs-tyrosine kinase 2 and JAK2, which can lead to phosphorylation and activation of STAT-4, as well as other STATs (Watford W T et al. Immunol. Rev 2004, 202, 139-156). In human NK cells, IL-15 can activate the binding of STAT1, STAT3STAT4, and STAT5 to the regulatory sites of the IFNG gene (Strengell M et al. J. Immunol. 2003, 170, 5464-5469). The activation of numerous transcription factors, including NF-κB, can affect the activation of IFNG transcription, Many of the synergistic stimuli that can enhance IL-12-mediated IFN-γ production by NK cells share the ability to activate the transcription factor NY-κB (Karman Y et al. Blood 2011, 117, 2855-2863). NF-κB is also a downstream mediator of TLR signaling, which can become activated in immune cells during injury and infections (Iwasaki A and Medzhitov R. Nat. Immunol. 2004, 5, 987-995; Napetschnig J and Wu H. Anna. Rev. Biophys, 2013, 42, 443-468; Hayden M S and Ghosh S, Genes .Dev. 2004, 18, 21952224).

Small-molecule natural product derivatives have been a productive source for the development of drugs. By 1990, >50% of all new drugs were either natural products or their analogs (Li J W and Vederas J C. Science 2009, 325, 161-165; Pan L et al. Phytochem. Lett. 2010, 3, 1-8), including those which act through immune modulation (Harvey A L. Drug Discov. Today 2008, 13, 894-901). This proportion has decreased in recent years, perhaps because the proportion of synthetic small molecules has increased, while performing the isolation of natural products from crude extracts is time-consuming and tabor-intensive; however, natural products and their analogs still account for >40% of newly developed drugs (Newman D J and Cragg G M. J. Nat. Prod. 2012, 75, 311-335; Pan L et al. Front. Biosci. (Schol. Ed) 2012, 4, 142-156), The popularity of developing drugs from natural products and their analogs is at least in part due to their relatively low side effects. Natural products provide enormous structural diversity, which also facilitates new drug discovery (Bindseil K U et al. Drug Discov. Today 2001, 6, 840-847).

Natural products were screened for their ability to enhance NK cell production of IFN-γ. It was found that phyllanthusmin C (PL-C, 4), a small-molecule lignan glycoside from plants in the genus Phyllanthus, can induce NK cell IFN-γ production in the presence or absence of monokines such as IL-12 and 1L-15. The induced NK cell activity resulted from enhanced TLR-NF-KB signaling. Interestingly, PL-C negligibly activated T cell IFN-γ production and also did not activate NK cell cytotoxicity. This selectivity of PL-C in immune activation can make it more suitable for development of a new clinically useful immune modulator.

Isolation of PBMCs and NK cells. Human PBMCs and NK cells were freshly isolated from leukopaks (American Red Cross, Columbus, Ohio) as described previously (He S et al, Blood 2013, 121, 4663-4671). PBMCs were isolated by Ficoll-Paque Plus (GE Healthcare Bio-Sciences, Pittsburgh, Pa.) density gradient centrifugation. NK cells (CD56⁺CD3⁻) were enriched with RosetteSep NK cell enrichment mixture (StemCell Technologies, Vancouver, BC, Canada). The purity of enriched NK cells was ≧80% assessed by flow cytometric analysis after staining with CD56-allophycocyanin and CD3-FITC Abs (BD Bio-sciences, San Jose, Calif.). These enriched NK cells were further purified with CD56 magnetic beads and LS columns (Miltenyi Biotec, Auburn, Calif.). The purity of magnetic bead-purified NK cells was ≧99.5%, as determined by the aforementioned flow cytometric analysis. CD56^{bright} and CD56^{dim} NK cell subsets were sorted by a FACS Aria II cell sorter (BD Biosciences) based on CDS6 cell surface density after staining with CD56-allophycocyanin and CD3-FITC Abs. The purity of CD56^{bright} and CD56^{dim} subsets was ≧99.0%. All human work was approved by The Ohio State University Institutional Review Board.

Cell culture and treatment. Primary NK cells, the NKL cell line and PBMCs were cultured or maintained in RPMI 1640 medium (Invitrogen, Carlsbad, Calif.), supplemented with 50 μg/mL penicillin, 50 μg/mL streptomycin, and 10% FBS (Invitrogen) at 37° C. in 5% CO₂. The NKL cell line is IL-2-dependent, and therefore, 150 IU/mL recombinant human IL-2 (Hoffman-LaRoche, Pendergrass. Ga.) was included in the culture, but cells were starved for IL-2 for 24 h prior to stimulation. For stimulation, cells were suspended at a density of 2.5×10⁶ cells/mL and seeded into a 6-well culture plate and rested for 1-2 h, followed by addition of stimuli. Cells were treated with PL-C in the presence or absence of IL-12 (10 ng/mL) or IL-15 (100 ng/mL) (R&D Systems, Minneapolis, Minn.) for 18 h or the indicated time. Cells were harvested for flow cytometric analysis or for RNA extraction to synthesize eDNA for real-time RT-PCR or for protein extraction to perform immunoblotting. Cell-free supernatants were collected to determine IFN-γ secretion by ELISA with commercially available mAb pairs (Thermo Fisher Scientific, Rockford, Ill.), according to the manufacturer's protocol as described previously (Yu J et al. Immunity 2006, 24, 575-590). To test whether PL-C also enhanced production when IL-12 or IL-15 were given at lower concentrations, purified primary NK cells were treated with 1, 0.1 ng/mL IL-12 or 10, 1 ng/mL IL-15 with or without 10 μM PL-C for 24 h. Supernatants were then harvested for IFN-γ ELISA. To study NF-κB involvement in PL-C-mediated enhancement of NK cell IFN-γ production, 10 μM NF-κB inhibitor N-tosyl-L-phenylalanine chloromethyl ketone (TPCK) was used to treat both purified primary NK cells or NKI, cells together with PL-C in the presence of IL-12, compared with no TPCK treatment. For TLR blocking assays, the purified NK cells were pretreated with 10 μg/mL anti-TLR1 (InvivoGen), anti-TLR3 (Hycult Biotech), anti-TLR6 (InvivoGen), or 10 μg/mL anti-TLR1 plus 10 μg/mL anti-TLR6 for 1 h prior to PL-C and/or IL-12 stimulation. Cells treated with the same concentration of nonspecific anti-IgG were used as control. The blocking Abs were also kept in the culture during the stimulation. For studying the effect of PL-C combined with ILR agonist, cells were treated with or without various concentration of Pam₃CSK₄ (TLR1/2 agonist) or FSL-1 (TLR6/2 agonist) for 18 h.

PL-C was isolated in chromatographically and spectroscopically pure form from the aboveground parts of plant Phyllanthus poilanei (Ren et al. J. Nat. Prod. 2014, 77, 1494-1504)

Intracellular flow cytometry. Intracellular flow cytometry was performed as described previously (Yu et al. Immunity 2006, 24, 575-590; Yu J et al. Blood 2010, 115, 274-281). Briefly, 1 μl/mL GolgiPlug (BD Biosciences) was added 5 h before cell harvest. After surface staining with CD3-FITC and CD56-allophycocyanin human Ahs (BD Biosciences), the cells were then washed and resuspended in Cytofix/Cytoperm solution (BD Biosciences) at 4° C. for 20 min. Fixed and permeabilized cells were stained with anti-IFN-γ-PE Ab (BD Bio-sciences). Labeled cells were used fora flow cytometric analysis. NK cells were gated on CD56⁺CD3⁻ cells, and CD4⁺ or CD8⁺ T cells were gated on CD56⁻ CD3⁺CD4⁺ or CD56⁻CD3+CD8⁺ cells, respectively. Data were acquired using an LSRII (BD Biosciences) flow cytometer and analyzed using FlowJo software (Tree Star, Ashland, Oreg.).

Real-time RT-PCR. Real-time RT-PCR was performed as described previously (Yu J et al. Immunity 2006, 24, 575-590; Yu J et al. Blood 2010, 115, 274-281). Briefly, total RNA from purified primary NK cells or NKL cells was isolated with a RNeasy kit (Qiagen, Valencia, Calif.). cDNA was synthesized from 1 to 3 total RNA with random hexamers (Invitrogen). Real-time RT-PCRs were performed as a multiplex reaction with the primer/probe set specific for IFNG GZMA (granzyme A), GZMB (granzyme B), PRE1 (perforin), Fasl (Fas ligand), and an internal control 18S rRNA (Applied Biosystems, Foster City, Calif.). mRNA expression of IL-12Rβ1 (IL-12Rβ1), IL-12Rβ2 (IL-12Rβ2), IL-15Rα (IL-15Rα), IL-15Rβ (IL-15Rβ), and HPRTI was detected by SYBR Green Master Mix (Applied Biosystems). The primers used are shown in Table 7. Expression levels were normalized to an 18S or HPRTI internal control and analyzed by the ΔΔCt method.

TABLE 7
Primers for real-time RT-PCR.

Target Gene

| | Primers | | |
|---|---|---|---|
| IFNG | For | 5′-GAAAAGCTGACTAATTATTCGGTAACTG-3′ | SEQ ID No. 1 |
| | Rer | 5′-GTTCAGCCATCACTTGGATGAG-3′ | SEQ ID No. 2 |
| GZMA | For | 5′-TCCTATAGATTTCTGGCATCCTCTC-3′ | SEQ ID No. 3 |
| | Rer | 5′-TTCCTCCAATAATTTTTTCACAGACA-3′ | SEQ ID No. 4 |
| GZMB | For | 5′-TCCTAAGAACTTCTCCAACGACATC-3′ | SEQ ID No. 5 |
| | Rer | 5′-GCACAGCTCTGGTCCGCT-3′ | SEQ ID No. 6 |
| Perforin | For | 5′-CAGCACTGACACGGTGGAGT-3′ | SEQ ID No. 7 |
| | Rer | 5′-GTCAGGGTGCAGCGGG-3′ | SEQ ID No. 8 |
| FasL | For | 5′-AAAGTGGCCCATTTAACAGGC-3′ | SEQ ID No. 9 |
| | Rer | 5′-AAAGCAGGACAATTCCATAGGTG-3′ | SEQ ID No. 10 |
| 18S | | 18S rRNA, PE Applied Biosystems, Foster City, CA | |
| HPRTI | For | 5′-CTTCCTCCTCCTGAGGAGTC-3′ | SEQ ID No. 11 |
| | Rer | 5′-CCTGACCAAGGAAAGCAAACG-3′ | SEQ ID No. 12 |
| IL12Rβ1 | For | 5′-ATGATGATACTGAGTCCTGCC-3′ | SEQ ID No. 13 |
| | Rer | 5′-GGAGCTGTAGTCGGTAAGTG-3′ | SEQ ID No. 14 |
| IL12Rβ2 | For | 5′-CTAAGCACAAAGCACCACTG-3′ | SEQ ID No. 15 |
| | Rer | 5′-CCGTTCCTTCCAGTATATCCT-3′ | SEQ ID No. 16 |
| IL15Rα | For | 5′-TCAAATGCATTAGAGACCCT-3′ | SEQ ID No. 17 |
| | Rer | 5′-TGCTTATCTCTGTGGTTCCT-3′ | SEQ ID No. 18 |
| IL15Rβ | For | 5′-GCAACATAAGCTGGGAAATCTC-3′ | SEQ ID No. 19 |
| | Rer | 5′-CGCACCTGAAACTCATACTG-3′ | SEQ ID No. 20 |

| | Probes | |
|---|---|---|
| IFNG | 5′-FAM-CTTGAATGTCCAACGCAAAGCAATACATGA-TAMRA-3′ | SEQ ID No. 21 |
| GZMA | 5′-FAM-CAGTTGTCGTTTCTCTCCTGCTAATTCCTGAAG-TAMRA-3′ | SEQ ID No. 22 |
| GZMB | 5′-FAMTGCTACTGCAGCTGGAGAGAAAGGCC-TAMRA-3′ | SEQ ID No. 23 |
| Perforin | 5′-FAMCCGCTTCTACAGTTTCCATGTGGTACACACTC-TAMRA-3′ | SEQ ID No. 24 |
| FasL | 5′-FAM-TCCAACTCAAGGTCCATGCCTCTGG-TAMRA-3′ | SEQ ID No. 25 |

Cytotoxicity assay. Cytotoxicity assay was performed as described previously (Yu J et al. Immunity 2006, 24, 575-590; Yu J et al. Blood 2010, 115, 274-281). Briefly, multiple myeloma cell line ARH-77 target cells were labeled with ⁵¹Cr and cocultured with purified primary NK cells, which were pretreated with or without 10 μM PL-C (phyllanthusmin C, 4) for 8 h in the presence of IL-12 (10 ng/mL) or IL-15 (100 ng/mL) prior to the coculture, at various E/T ratios in a 96-well V-bottom plate at 37° C. for 4 h. At the end of coculture, 100 μl supernatants were harvested and transferred into scintillation vials with a 3-mL liquid scintillation mixture (Fisher Scientific). The release of ⁵¹Cr was counted on a TopCount counter (Canberra Packard, Meriden, Conn.). Target cells incubated in 1% SDS or complete medium were used to determine maximal or spontaneous ⁵¹Cr release. The standard formula of 100×(cpm experimental release−cpm spontaneous release)/(cpm maximal release−cpm spontaneous release) was used to calculate the percentage of specific lysis.

Immunoblotting. Immunoblotting was performed as described previously (Yu J et al. Immunity 2006, 24, 575-590; Yu J et al. Blood 2010, 115, 274-281). The equal number of cells from each sample was directly lysed in 2× Laemmli buffer (Bio-Rad, Hercules, Calif.) supplemented with 2.5% 2-ME, boiled for 5 min, and subjected to immunoblotting analysis as described previously (Yu J et al. Immunity 2006, 24, 575-590). Abs against p65, p-p65, p-STAT3, p-STAT4, p-STATS, STAT3, STAT4, STAT5 (Cell Signaling Technology, Danvers, Mass.), and T-BET (Santa Cruz Biotechnology, Santa Cruz, Calif.) were used fbr immunoblotting. Immunoblotting with Abs against (3-actin (Santa Cruz Biotechnology) served as an internal control.

EMSA. Nuclear extracts were isolated using a nuclear extract kit, according to the manufacturer's instruction (Active Motif, Carlsbad, Calif.). EMSA was performed as described previously (Bachmeyer C et al. Nucleic Acids Res, 1999, 27, 643-648). Briefly, a ³²P-labeled double-stranded oligonucleotide, 5′-GGGAGGTACAAAAAAATTTCCAGTCCTTGA-3′ (SEQ ID No. 26), containing an NF-κB binding site C3-3P (−278 to −268) of the IFNG promoter (Sica A et al. J. Biol. Chem. 1997, 272, 30412-30420), was incubated with nuclear extracts (2 μg) for 20 min before resolving on a 6% DNA retardation gel (Invitrogen). After electrophoresis, the gel was transferred onto filter paper, dried, and exposed to x-ray films. In Ab gel supershift assays, p65 Abs (Rockland Immunochemicals, Gilbertsville, Pa.) were added to the DNA-protein binding reactions after incubation at room temperature for 10 min, followed by an additional incubation for 20 min before gel loading.

Chromatin immunoprecipitation. Chromatin immunoprecipitation (ChIP) assay was carried out with a ChIP assay kit (Upstate Biotechnology, Lake Placid, N.Y.), according to the manufacturer's protocol. An equal amount (10 ng) of rabbit monoclonal anti-p65 Abs or normal rabbit IgG Abs (Cell Signaling Technology) was used to precipitate the cross-linked DNA/protein complexes. The sequences of primers spanning the diMrent NF-κB sites on the IFAIG promoter have been described previously (Kannan Y et al. Blood 2011, 117, 2855-2863). DNA precipitated by the anti-p65 or the normal IgG Abs was quantified by real-time PCR, and values were normalized to input DNA.

TLR activation assessment. Human embryonic kidney 293T (HEK293T cells were cotransfected with TLR1 or 6 expression plasmids (0.5 μg for each) for 24 h along with 1GL-3κB-Luc (1 μg), which contains three tandem repeats of κB site (Guttridge D C et al. Mol. Cell. Biol. 1999, 19, 5785-5799), and pRL-TK renitla-luciferase control plasmid (5 ng; Promega). The cells were then treated with various concentrations of PL-C for additional 24 h after replacing old medium with fresh medium. Firefly and renilla luciferase activities were detected by using Dual-Luciferase Reporter Assay System (Promega , and the ratio of firefly/renilla luciferase activities was used to determine the relative activity of NF-κB.

TLR1 short hairpin RNA knockdown in NKL cells. A TLR1 short hairpin RNA (shRNA) plasmid was constructed by inserting RNA interference sequence (5′-GTCTCATCCACGTTCCTAAT-3′ (SEQ ID No. 27)) into GFP expressing pSUPER-retrovirus vector. Viruses were prepared by transfecting the shRNA plasmid and packaging plasmids into phoenix cells. Infection was performed as follows: NKL cells were cultured in virus-containing medium and centrifuged at 1800 rpm at 32° C. for 45 min and then incubated for 2-4 h at 32° C. This infection cycle was repeated twice. GFP-positive cells were sorted on a FACSAria II cell sorter (BD Biosciences). Knockdown of TLR1 in the sorted NKL cells was confirmed by real-time RT-PCR.

Statistical analysis. An unpaired Student t test was used to compare two independent conditions such as PL-C versus control) fbr continuous endpoints, Paired t test was used to compare two conditions with repeated measures from the same donor. A one-way ANOVA model was used for multiple comparisons. A two-way ANOVA model was used to evaluate the synergistic effect between IL-12 or IL-15. and PL-C. The p values were adjusted for multiple comparisons using Bonferroni method. All tests are two-sided. A p value 0.05 was considered statistically significant.

### Results

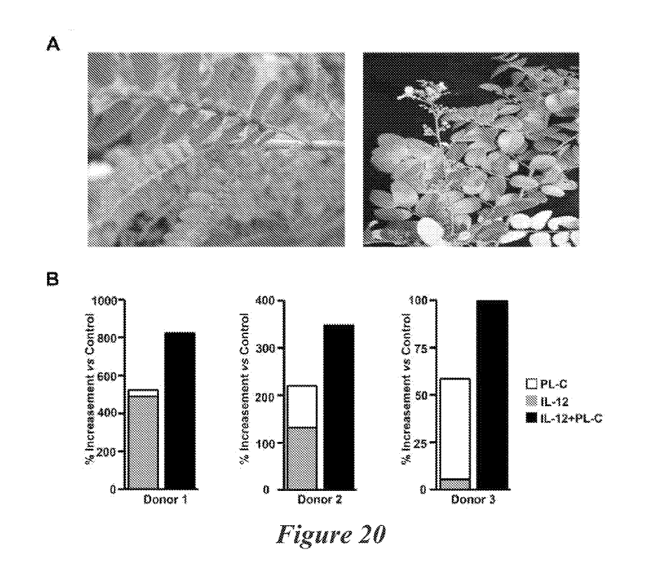

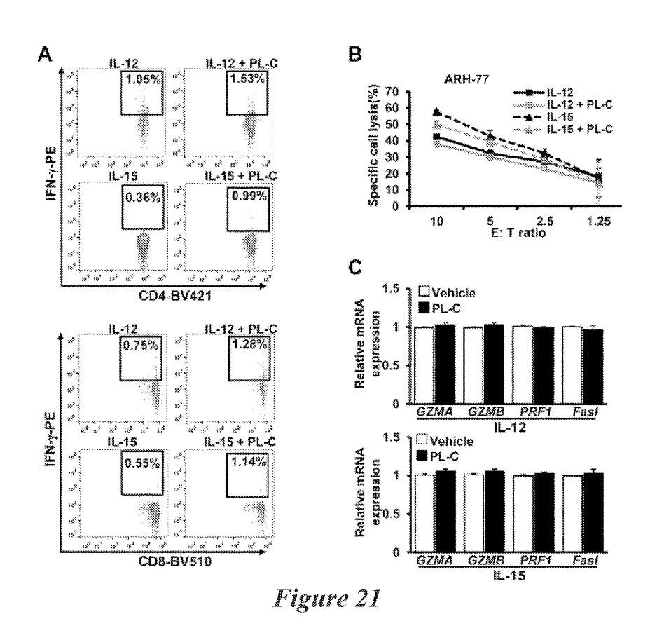

Over 50 candidate compounds isolated from edible or nonedibie plants (e.g., curcumin, β-glucan, etc) were screened for their capacity to enhance human NK cell activity. A diphyllin lig,nan glycoside, PL-C (phyllanthusmin C; FIG. 19A), which can be isolated from both edible (e.g., Phyllanthus retiatiatas) and nonedible (e.g., Phyllanthus poilanei) plants of the Phyllanthus genus collected from parts of Asia (FIG. 20A) (Ren et al. J. Nat. Prod. 2014, 77, 1494-1504; Ma J X et at J. Asian Nat. Prod. Res. 2012, 14, 1073-1077; Jansen P C M, Plant Resources of Tropical Africa Program. 2005, Dyes and tannins. PROTA Foundation, Wageningen, Netherlands), was able to enhance IFN-γ production by NK cells. When total PBMCs from healthy donors were cultured with PL-C in the presence of the cytokines IL-12 or IL-15 (stimulators of IFN-γ NK cells, each constitutively expressed in vivo) (Stevceva et al. Lett. Drug Des. Diseov. 2006, 3, 586-592), intra-cellular staining for IFN-γ protein assessed via flow cytometric analysis indicated that NK cell IFN-γ production was increased (FIG. 19B, left panel). PL-C also enhanced IFN-γ production in enriched INK cells in the presence of IL-12 or IL-15 (FIG. 19B, right panel). To determine whether PL-C directly or indirectly acts on NK cells to enhance their IFN-γ production, NK cells were purified (purity 99.5%) from total PBMCs via FACS and the level of IFN-γ secretion from the purified NK cells was measured using ELISA. PL-C induced NK cell secretion of IFN-γ even in the absence IL-12 or IL-15 (FIG. 37C, ieft panel). PL-C also enhanced NK cell IFN-γ secretion in the presence of IL-12 or IL-15 stimulation (FIG. 19C, middle and right panels). Statistical analysis indicated a synergistic effect of IL-12 and PL-C IFN-γ expression (FIG. 20B). The data also showed that PL-C induced IFN-γ gene (IFNG) expression at the transcriptional level regardless of whether it was added alone or in the presence of IL-12 or IL-15 (FIG. 19D). PL-C also promoted IFN-γ production in purified primary NK cells when tested at a much lower concentrations of IL-12 (1 and 0.1 ng/mL) or IL-15 (10 and 1 ng/mL) (FIG. 19E). Increased IFN-γ secretion and IFNG mRNA transcription were found in the IL-γ-dependent NK cell line, NKL (FIG. 19F). When PBMCs were used, the majority of IFN-γ-producing cells were found to be NK cells, whereas there were few, if any, CD4⁺ or CD8⁺ T cells responding to PL-C stimulation in combination with IL-12 or IL-15 (FIG. 21A). PL-C showed limited effect on NK cytotoxicity against the K562 cell line or multiple myeloma cell ARH-77 (FIG. 21B) and MM.1S, regardless of whether cells were incubated in media alone, with IL-12, or with IL-15, Consistent with this, expression of cytotoxicity-associated genes such as granzyme A, granzyme B, perforin, and Fas ligand were also unaffected by PL-C when costimulated with IL-12. or IL-15 (FIG. 21C).

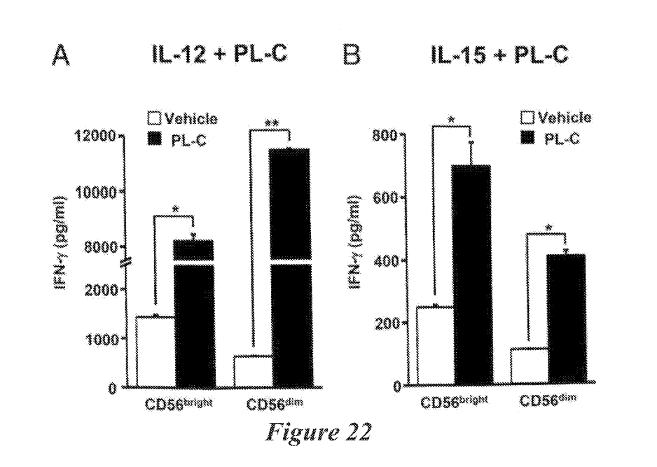

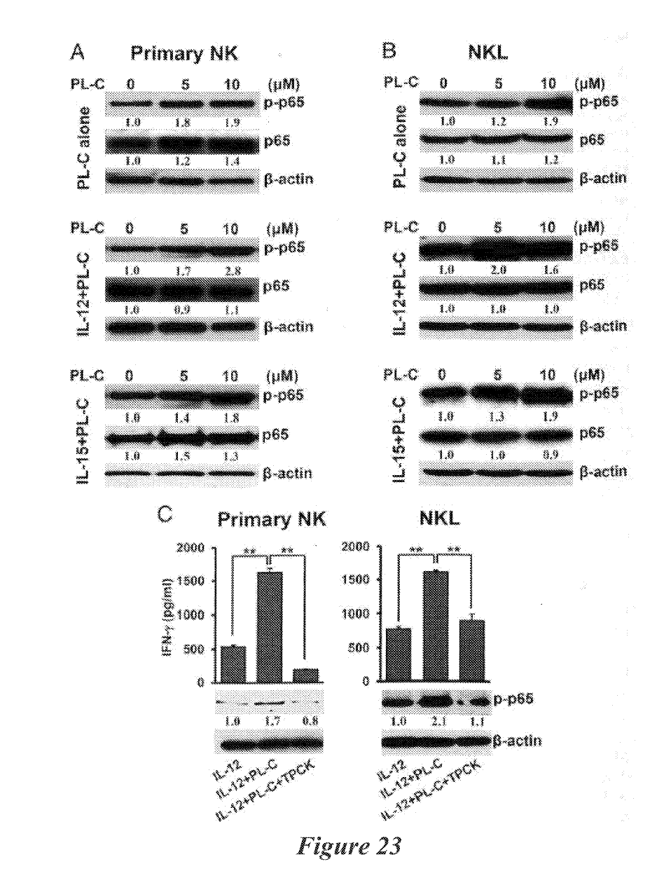

Based on the relative density of CD56 surface expression, mature human NK cells can be phenotypically divided into CD56^{bright} and CD56^{dim} subsets. Human peripheral blood NK cells are composed of ˜10% CD56^{bright} NK cells and 90% CD56^{dim} NK cells (Caligiuri M A Blood 2008, 112, 461-469). Cytokine-activated CD56^{bright} NK cells can proliferate and secrete abundant IFN-γ but display minimal cytotoxic activity at rest; in contrast, CD56^{dim} NK cells have little proliferative capacity and produce negligible amounts of cytokine-induced IFN-γ but are highly cytotoxic at rest (Caligiuri M A Blood 2008, 112, 461-469). During costimulation with IL-12 or IL-15, IFN-γ secretion from both CD56^{bright} and CD56^{dim} NK cells was enhanced by PL-C when compared with parallel cultures treated with a vehicle control (FIGS. 22A and B). In some donors, the CD56^{dim} NK cells produce more IFN-γ than CD56^{bright} NK cells when costimulated with PL-C and IL-12, as previously reported when NK cells recognize tumor cells (Zhang X and Yu J. Blood 2010, 115, 2119-2120; Fauriat C et al. Blood 2010, 115, 2167-2176).

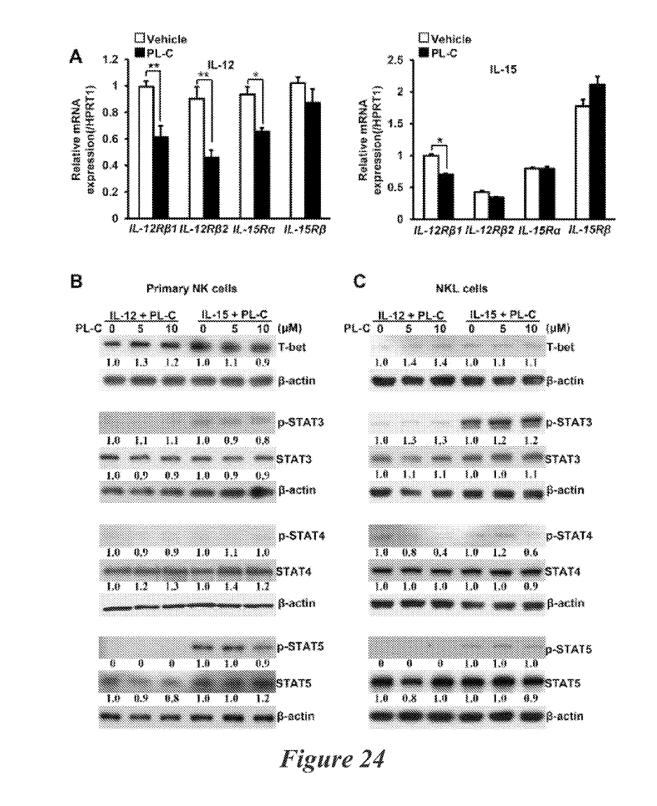

Cytokine-induced IFN-γ production can occur through the JAK-STATs, T-BET, MAPK, or NF-κB signaling pathways (Schoenborn J R and Wilson C B. Adv. Immunol. 2007, 96, 41-101). Transcription factors in these signaling pathways can be associated with corresponding binding sites in the regulatory elements of the IFNG gene, subsequently enhancing IFND mRNA synthesis (Schoenborn J R and Wilson C B. Adv. Immunol. 2007, 96, 41-101.). Which of these signaling pathways participate in the PL-C-rnediated. IFN-γ induction in NK cells was therefore determined. NF-κB p65 phosphorylation increased upon stimulation of primary NK cells and the NKL cell line with PL-C alone, whereas the level of total p65 was less or negligibly changed (FIGS. 23A and B, upper panels). An increase of p65 phosphorylation but not total p65 was also observed when primary NK cells or NKL cells were treated with PL-C in the presence of IL-12 or IL-15 (FIGS. 41A and B, middle and bottom panels). No significant change in the level ofp65 transcript was observed in primary NK cells and NKL, cells. Next, whether PL-C affects IL-12R or .IL-15R and their downstream STAT signaling pathways was assessed. When cotreated with IL-12, PL-C downregulated mRNA expression of IL-12Rβ1, IL-12Rβ2, and IL-15Rα. However, when cotreated with IL-15, PL-C had no obvious effect on all IL-12R. or IL-15R, except for a moderate downregulation of IL-12Rβ1 (FIG. 24A). No upregulation of total or phosphorylated STAT3, STAT4, and STAT5 in either purified primary NK or NKL cells was observed. There was no significant change of T-BET in either purified primary NK cells or NKL cells being treated with PL-C (FIGS. 24B and C). To further explore NF-κB involvement in PL-C-mediated enhancement of NK cell IFN-γ production, the NF-κB inhibitor TPCK was used, because it has been shown to directly modify thiol groups on Cys-179 of inhibitory K-B kinase (IKKβ) and Cys-38 of p65/ReIA, thereby inhibiting NF-κB activation (Ha K H et al. Biochemistry 2009, 48, 7271-7278). In purified primary NK cells and the NM, cell line, TPCK indeed inhibited PL-C-induced p65 phosphorylation, which was correlated with an inhibition of PL-C-induced IFN-γ secretion (FIG. 23C).

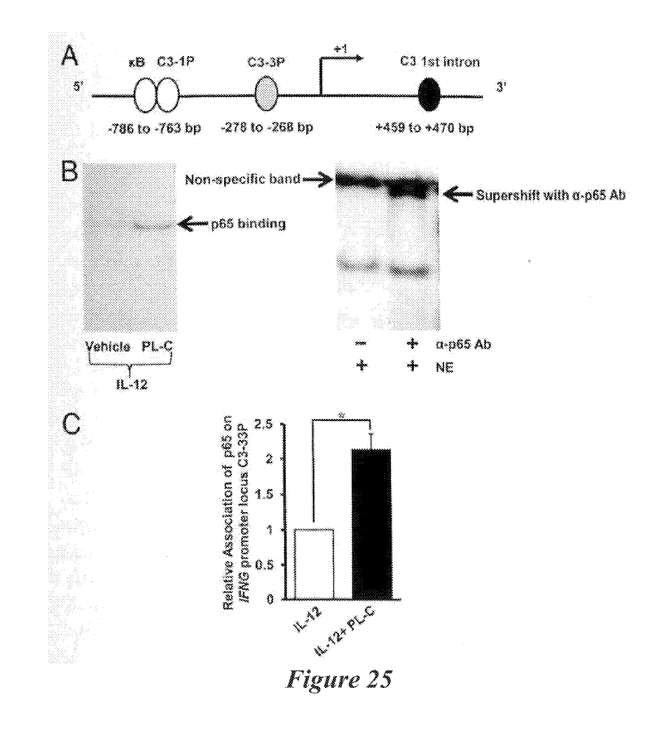

As PL-C (phyllanthusmin C, 4) can induce NF-κB activity and enhance IFN-γ production in NK cells, next whether PL-C would facilitate the binding of NF-κB to the promoter of the IFNG gene in these cells was investigated. Four different NF-κKB binding sites at the IFNG locus (κB, C3-1P, C3-3P, and C3 first intron) have been reported previously (FIG. 25A) (Sica A et al. J. Biol. Chem. 1997, 272, 30412-30420). EMSA using a ³²P-labeled oligonucleotide containing the C3-3P NF-κB binding site of the IFNG promoter indicated that nuclear extracts prepared from purified primary NK cells treated with PL-C and IL-12 formed more DNA-protein complexes than those treated with IL-12 alone (FIG. 25B, left panel). The presence of p65 in the DNA-protein complexes was demonstrated by Ab gel supershift assay using anti-p65 Abs (FIG. 25B, right panel). To find physiologically relevant evidence that PL-C augmented binding of p65 to the IFNG promoter, a ChIP assay was undertaken. Using primary NK cells purified from healthy donors, it was found that treatment with PL-C in the presence of IL-12 enhanced p65 binding to the C3-3P binding site on the IENG promoter when compared with IL12-treated primary NK cells (FIG. 25C). ChIP assays among different donors showed no consistent results that PL-C induced binding of p65 to the previously characterized κbB, C3-1P and C3 first intron NF-κB binding sites on the IFNG promoter.

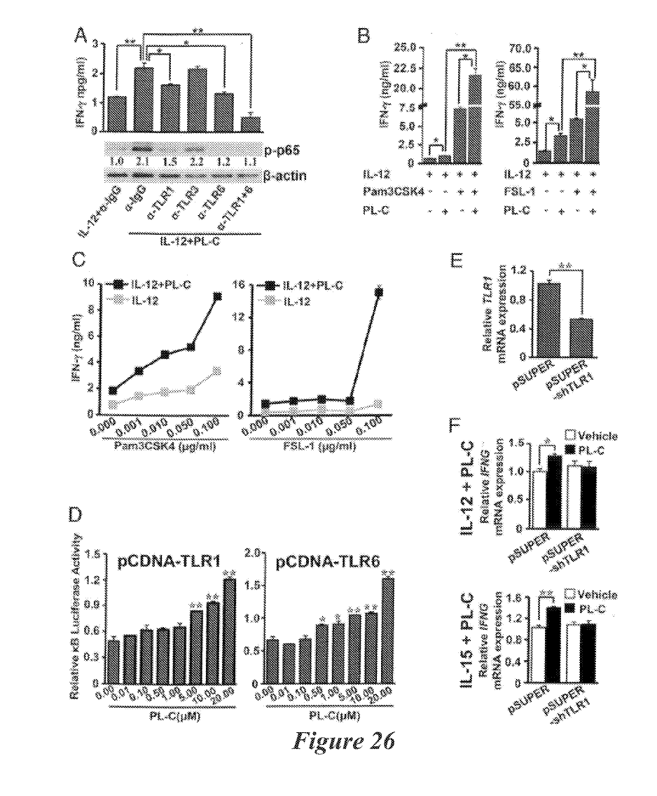

TLR signaling is upstream of NF-κB signaling, and activation of TLRs can lead to a robust downstream TLR/IRAK-2/NF-κB-mediated induction of cytokine gene expression in immune cells (Hayden M S and Ghosh S. Genes Dev. 2004, 18, 21952224). Therefore, next whether TLRs mediated PL-C-induced IFN-γ production by human NK cells was determined. Human NK cells can express TLR1, TLR3, and TLR6 (He S et al. Blood 2013, 121, 4663-4671; Hornung V et al. J. Immunol. 2002, 168, 4531-4537). The experiment started by Ab blocking these TLRs. it was found that blockade of TLR1 or TLR6 in primary NK cells reduced PL-C-mediated induction of IFN-γ, whereas blockade of TLR3 had no significant effect on NK cell activation. Combined blockade of TLR1 and TLR6 reduced PL-C-enhanced NK cell IFN-γ expression to levels lower than those seen with blockade of either TLR1 or TLR6 (FIG. 26A, top panel). To determine whether the effect of blocking Abs PL-C-induced IFN-γ production is likely mediated through the NF-κB signaling pathway, the phosphorylation level of p65 induced by PL-C in the presence and absence of the TLR blocking Abs was examined. Consistently, blockade of TLR1 and/or TLR6 also inhibited PL-C-induced phosphorylation of p65, suggesting that PL-C-induced IFN-γ production can occur at least in part through the TLR1/6-NF-κB signaling pathway (FIG. 26A, bottom panel). Whether PL-C could affect the expression of TLR1 and TLR6 was also examined. No obvious changes in TLR1 or TLR6 gene expression were observed after treatment with PL-C alone or in the presence of IL-12. To further explore whether PL-C would augment TLR-mediated IFN-γ induction, NK cells were treated with 10 μM PL-C combined with a ligand of each of the two aforementioned TLRs in the presence of IL-12. PL-C enhanced IFN-γ production induced by Pam₃CSK₄ (TLR1/2 ligand) or FSL-1 (TLR6/2 ligand) in the presence of IL-12 when the ligands were at the concentration of 1 μg/mL (FIG. 26B). It was also found that PL-C enhanced Pam₃CSK₄- and FSL-1-induced IFN-γ production in the presence of IL-12 when the ligands were added at various concentrations <1 uglinL (FIG. 26C). To further confirm that PL-C activates the TLR-NF-κB signaling pathway, TLR1 or TLR6 was cotransfected with pGL-3KB-Luc and control plasmid pRL-TK renilla-luciferase plasmids. PL-C treatment was found to induce luciferase reporter activity in a dose-dependent fashion, suggesting an increase of NF-κB binding to the KB binding sites (FIG. 26D). TLR1 expression was knocked down in NKL cells by using TLR1 shRNA to validate that TLR1 signaling participated in PL-C-mediated enhancement of NK cell IFN-γ production. After confirming TLR1 was successfully knocked down in TLR1 shRNA NKL cells with ˜50% TLR1 mRNA inhibition (FIG. 26E), it was found that the increase in IFN-γ production mediated by PL-C vanished when cotreated with IL-12 or IL-15 in these cells (FIG. 26F). These data suggest that PL-C directly activates TLR-NF-κB signaling pathway to enhance IFN-γ production in NK cells.

NK cells are a lymphocyte subset that can destroy tumor cells and clear viral infections upon first encounter (Caligiuri M A Blood 2008, 112, 461-469). Enhancement of NK cell activity for prevention or treatment of cancer and viral infection is of interest in the field of immunology. NK cell activation can be achieved through exposure to cytokines such as IL-2 (Wang K S et al. Blood 2000, 95, 3183-3190) and IL-12 (Robertson M J et al. J. Exp. Med. 1992, 175, 779-788; Chehimi J et al. J. Exp. Med. 1992, 175, 789-796). However, this approach has had limited success in part because of the toxicity resulting from the systemic administration of these cytokines (Rosenberg S A et al. Ann. Surg. 1989, 210, 474484, discussion 484-485) and the pleotropic effects of these agents. One example of the latter is that IL-2 can induce expansion of regulatory T cells (Gowda A et al. MAbs 2010, 2, 35-41; Shah M H et al. Clin. Cancer Res. 2006, 12, 3993-3996), which in turn can dampen NK cell functions (Ralainirina N et al. J. Leakoc. Biol. 2007, 81, 144-153). An agent that can produce a modest induction of NK function with relative specificity among immune effector cells would be useful for prevention of cancer or infection in susceptible individuals.

PL-C (phyllanthusmin C, 4), a diphyllin lignan glycoside, which can be isolated from both edible and nonedible plants of the Phyllanthus genus, can specifically enhance IFN-γ production by human NK cells, as shown herein above. Mechanistically, P LC can sense TLR1 and/or TLR6 on human NK cells, which in turn can activate the NF-κB subunit p65 to bind to the proximal region of the IFNG promoter. PL-C has only negligible effects on T cell effector function, which is consistent with higher expression of TLR1 and TLR6 NK cells than in T cells (Hornung V et al. J. Immunol. 2002, 168, 4531-4537). This can increase the likelihood that pl.eiotropic effects on immune activation and systemic toxicity of the agent might be limited.

Targeting NK cells can be used to prevent cancer. An 11 year follow-up population study of 3625 people 40 years of age demonstrated that the potency of peripheral blood NK cells for lysing tumor cell targets was inversely associated with cancer risk (Imai K et al. Lancet 2000, 3.56, 1795-1799). Moreover, as cancer susceptibility increases with age, NK cell potency subsides with age (Hazeldine J et al. Ageing Res. Rea 2013, 12, 1069-1078; Shaw A C et al. Curr. Opin. Immunol. 2010, 22, 507-513). NK cell activity is correlated with relapse-free survival in some cancer patients (e.g., those with acute myeloid leukemia, AML) (Tajima F et al. Leukemia 1996, 10, 478-482). NK cells can be tools used to control tumor development at the early stages, as they can play a role in tumor surveillance. Once cancer is established, tumor cells can inactivate immune cells, including NK cells, which can result in an immunosuppressive microenvironment (Jewett A and Tseng H C.J. Cancer 2011, 2, 443-457). Indeed, NK cell function is found to be anergic or impaired in various types of cancer (Critchley-Thome R J et al. Proc. Natl. Acad. Sci. USA 2009, 106, 9010-9015; Guiliot B et al. Br. J. Dermatol. 2005, 152, 690-696). Moreover, at the later stages of cancer development, the immune system, including NK cells and IFN-γ, can edit tumor cells and facilitate their escape from immune destruction (Ikeda H et al. Cytokine Growth Factor Rev. 2002, 13, 95-109; Dunn G P et al. Nat. Immunol. 2002, 3, 991-998; O'Sullivan T et al. J. Exp. Med.2012, 209, 1869-1882). Therefore, NK cells can play a role in prevention of cancer, and their selective modulation can be important in this scenario.

The findings discussed herein provide an avenue to prevent or treat cancer using natural products and their derivatives through enhancing NK cell immuno-surveillance. PL-C (phyllanthusmin C, 4) is likely relatively safe, compared to cytokines, as supported by the lack of substantial toxicities observed in mice treated with up to 500 mg PL-C/kg body weight. Developing less toxic drugs is important for preventing or treating some cancers, especially thr those which are dominant in children or in elderly populations, such as AML AML primarily affects older adults: the median age at diagnosis is >65 years (Estey E and Milner H. Lancet 2006, 368, 18941907; Yanada M and Naoe T. Int. J. Hematol. 2012, 96, 186-193). The 5 year survival rate of AML in older adults remains under 10% (Stein E M and Tallman M S. Int J. Hematol 2012, 96, 164-170). Elderly AML patients are less able than younger patients to tolerate effective therapies such as intensive chemotherapy and allogeneic stem cell transplantation.

PL-C (phyllanthusmin C, 4) can selectively activate NK cells through regulating production of cytokines, such as IFN-γ. Therefore, in vivo, PL-C can achieve its cancer prevention or treatment effects through increasing NK cell IFN-γ secretion to activate other innate immune components, such as macrophages (Nathan C F et al. J. Exp. Med. 1983, 158, 670-689), as well as adaptive immune components, such as CD8⁺ T cells (Kos F J and Engleman E G. J. Immunol. 1995, 155, 578-584; Ge M Q et al. J. Immunol. 2012, 189, 2099-2109). Unlike cytokine stimulation, which can induce both IFN-γ production and cytotoxicity, the selective induction of NK cell IFN-γ production by PL-C can provide an opportunity to separate the two major functions of NK cytokine production and cytotoxicity, especially when cytotoxicity may cause damage to normal tissues (e.g., in the graft-versus-host disease and pregnancy contexts). This separation naturally exists in the human immune system, as CD56^{bright} and CD56^{dim} NK cells have differential functions in terms of IFN-γ production and cytotoxicity, and some tissues and/or organs predominantly have only one of these subsets. For example, the lymph nodes (Fehniger T A et al. Blood 2003, 101, 3052-3057) and the uterus (King A et al. Am. J. Reprod. Immunol. 1996, 35, 258-260) almost exclusively contain CD56^{bright} NK cells.

Mechanistically, it was found that PL-C can sense TLR1 and TLR6 to activate NF-κB signaling in NK leading to the enhancement of IFN-γ production. In support of this, knockdown of TLR1 by shRNA eliminated the effect of IFN-γ production in NKL cells. It has previously been demonstrated that TLR1 and TLR6 can be expressed in human NK cells (He S et al. Blood 2013, 121, 4663-4671; Hornung V et al. J. Immunol. 2002, 168, 4531-4537). TLR1 and TLR6 share 56% amino acid sequence identity (Jin M S et al. Cell 2007, 130, 1071-1082) and both can complex with TLR2 to recognize bacterial lipoproteins and iipopeptides (Hopkins P A and Sriskandan S. Clin. Exp. Immunol. 2005, 140, 395-407). TLR1 and TLR6 can thus possess a common binding site for PL-C. PL-C also activates TLR1 and TLR6 downstream NF-κB signaling in NK cells, and transfection of TLR1 or TLR6 induces NF-κB reporter activity. Moreover, the data suggest that PL-C can either lower the threshold for or synergize with TLR1 and TLR6 ligands to activate NK cells.

In summary, PL-C (phyllanthusmin C, 4) can effectively stimulate NK cells to secrete IFN-γ. PL-C can act through TLR1 and TLR6, which subsequently can activate NF-κB signaling to induce binding of p65 to the proximal region of the IFNG promoter NK cells.

### Example 5

A sample of the aerial parts of Phyllanthus songboiensis N. N. Thin was collected. from an erect, free-standing plant 75 cm tall on the shore of Lake Kego (18° 09.033′ N; 105° 59,843′ E), Kego Nature Reserve, Cam Xuyen District, Hatinh. Province, Vietnam, in December, 2008, by D.D.S., T.N.N., and V.T.T. A voucher herbarium specimen (DDS 14285) representing this collection has been identified by D.D.S. and is deposited at the John G. Searle Herbarium of the Field Museum of Natural History, Chicago, Ill., under the accession number FM 2287532. Pham Hoang Ho, 2000, entry No, 4733d.

A sample of the milled, air-dried aerial parts of P. sangboiensis (699 g) was extracted with MeOH (2 L×6, 24 h each) at room temperature. The solvents were evaporated in vacua, and the dried MeOH extract (71.0 g, 10.1%) was resuspended in 600 mL of MeOH mixed with H₂O (H₂O:MeOH, 10:90, v/v) and partitioned with n-hexane (500, 400, and 300 mL) to yield a n-hexane-soluble residue (D1, 7.0 g, 1.0%). The aqueous MeOH layer was then partitioned with CHCl₃ (500, 300, and 300 mL) to afford a CHCl₃-soluble extract (D2, 3.0 g, 043%), which was followed by washing with a 1% aqueous solution of NaCl to partially remove plant pol:mhenols. The n-hexane-soluble extract showed low cytotoxicity toward the HT-29 cell line (IC₅₀, 15.0 μg/mL), with the CHCl₃-soluble extract more active in this regard (IC₅₀, 4.4 μg/mL). The water-soluble extract was inactive (IC₅₀>20 μg/mL) in this bioassay system.

The n-hexane-soluble extract (6.8 g) was subjected to silica gel CC (4.5×45cm), eluted with gradient mixtures of n-hexaneacetone (100:1→1:1; 500 mL each). The eluates were pooled by TLC analysis to give five combined fractions. Of these, active fractions 4 (IC₅₀, 9.7 μg/mL) and 5 (IC₅₀, 6.8 μg/mL) were combined and applied to another silica gel-containing column (2.5×20 cm), eluted with gradient mixtures of n-hexane-acetone (20:1→3:1, 200 mL each). Fractions were pooled by TLC analysis to give 16 combined fractions (D1F4F1-D1F4F16). Of these, fractions D1F4F5-D1F4F7 were combined and subjected to silica gel CC, eluted with a gradient of n-hexane-acetone and then purified by separation over a Sephadex LH-20 column, eluted with CH₂Cl₂-MeOH (1:1), affording (−)-spruccanol (1.5 mg). Fractions D1F4F10-D1F4F13 were combined and applied to a silica gel column, eluted with a gradient of n-hexane-acetone and then purified by separation over a Sephadex LH-20 column, eluted with CH₂Cl₂-MeOH (1:1), furnishing (−)-β-sitosterol-3-O-β-D-(6-O-palmitoyl)glucopyranoside (6 mg) and (−)-pinoresinol (1 mg).

The CHCl₃-soluble extract (2.8 g, IC₅₀, 4.4 μg/mL) was subjected to silica gel CC (2.5×45 cm) by elution with a gradient of n-hexane-acetone. Fractions were pooled by TLC analysis to give 15 combined fractions (D2F1-D2F15). Of these, fractions D2F8, D2F9, D2F11, and D2F12 were found to be active, with IC₅₀ values of 16.7, 15.5, 16.4, and 7.1 μg/mL, respectively. Fraction D2F8 was applied to a silica gel column, eluted with a gradient of n-hexane-acetone and then purified by separation over a Sephadex LH-20 column, eluted with CH₂Cl₂-MeOH (1:1), affording (+)-songbosin (6 mg). Fraction D2F9 was subjected to silica gel CC, eluted with a gradient of n-hexane-acetone and then finally purified by separation over a Sephadex LH-20 column, eluted with CH₂Cl₂-MeOH (1:1), furnishing (+)-songbodichapetalin (5 mg) and (−)-7′-hydroxydivanillyltetrahydrofuran (2 mg). Fractions D2F11 and D2F12 were applied to a silica gel column, eluted with a gradient of n-hexane-acetone and next purified by separation over a Sephadex LH-20 column, eluted with CH₂Cl₂-MeOH (1:1), affording (+)-acutissimatignan A (9, 2 mg), (−)-syringaresinol (3 mg), and (+)-secoisolariciresinol (2 mg).

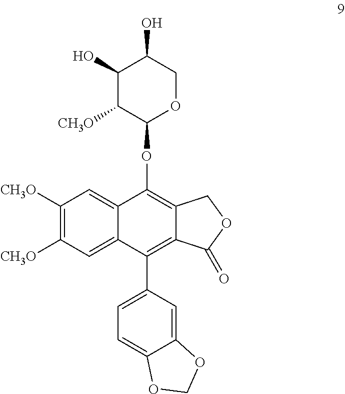

Interfacial inhibition or poisoning of topo IIα can be evaluated by trapping topo II-plasmid DNA covalent complexes with sodium dodecyl sulfate, digesting away the enzyme, and releasing cleaved linear DNA. The topo IIα-inhibitory activity of etoposide and compound 9 was assessed using a procedure reported previously (Hasinoff, B. B. et al., Mol. Pharmacol. 2005, 67, 937-947; Ren, Y. et al. J. Nat. Prod. 2014, 77, 1494-1504). In brief, assay buffer containing pBR322 DNA and test compound/DMSO were mixed and allowed to sit at room temperature for 30 min after which topo IIα was added to initiate the reaction. The tubes were incubated at 37° C. for 15 min, and then quenched with 1% (v/v) SDS/10 mM disodium EDTA/200 mM NaCl. The mixture was treated subsequently with 0.77 mg/ml proteinase K (Sigma, St. Louis, Mo., USA) at 55° C. for 60 min to digest the protein, and DNA bands were separated by electrophoresis (18 h at2 V/cm) on an agarose gel (1.3% w/v) containing 0.7 μg/ml ethidium bromide. Then, DNA in the gel was imaged by its fluorescence on a Chemi-Doc XRS+ imager (Bio-Rad, Hercules, Calif., USA). Percent linear produced was quantified from total fluorescence of all bands accounting for differences in relative fluorescence of the different forms of DNA as previously reported (Projan, S. J. et al., Plasmid 1983, 9, 182-190).

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods and combinations of various features of the compounds and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

What is claimed is:

1. A method of treating or preventing cancer in a subject, or causing immunostimulation in a subject, comprising administering to the subject an effective amount of a composition comprising a compound of Formula VI: 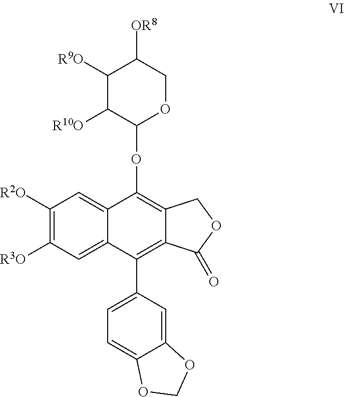
wherein
R² and R³ are independently hydrogen, halogen, substituted or unsubstituted C₁-C₄ alkyl, substituted or unsubstituted C₁-C₄ atkoxycarbonyl, hydroxycarbonyl, substituted or unsubstituted acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio; or R² and R³ taken together with the atoms to which they are attached thrm a substituted or unsubstituted 5 to 7 membered heterocyclic moiety;
R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

2. The method of claim 1, wherein R² is a water solubilizing group.

3. The method of any one of claims 1-2, wherein R³ is a water solubilizing group.

4. The method of any one of claims 1-3, wherein R² and R³ are independently hydrogen, substituted or unsubstituted C₁-C₄ alkyl, or substituted or unsubstituted phosphonyl.

5. The method of any one of claims 11-4, wherein R² and R³ are independently hydrogen, CH₃, or PO₃H₂.

6. The method of any one of claims 1-5, wherein R⁸ is a water solubilizing group.

7. The method of any one of claims 1-6, wherein R⁹ is a water solubilizing group.

8. The method of any one of claims 1-7, wherein R¹⁰ is a water solubilizing group.

9. The method of any one of claims 1-8, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

10. The method of any one of claims 1-9, wherein R⁸, R⁹ and R¹⁰ are independently hydrogen, CH₃, C(O)CH₃, or PO₃H₂.

11. The method of airy one of claims 1-10, wherein the con pound is of Formula VI-A: 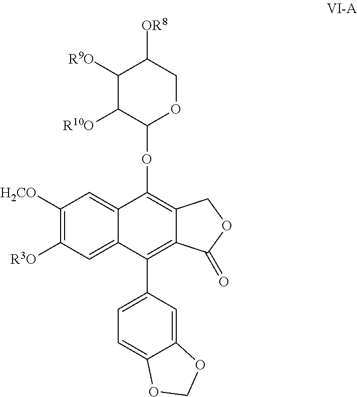
wherein
R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted. C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfnyl, substituted or unsubstituted sulfonyl, substituted or unsubstituted sulfonamide, or substituted or unsubstituted thio;
or a pharmaceutically acceptable salt or prodrug thereof.

12. The method of claim 11, wherein R³ is a water solubilizing group.

13. The method of any one of claims 11-12, wherein R³ is hydrogen, substituted or unsubstituted C₁-C₄ alkyl, or substituted or unsubstituted phosphonyl.

14. The method of any one of claims 11-13 wherein R³ is hydrogen, CH₃, or PO₃H₂.

15. The method of any one of claims 11-14, wherein R⁸ is a water solubilizing group.

16. The method of any one of claims 11-15, wherein R⁹ is a water solubilizing group.

17. The method of any one of claims 11-16, wherein R¹⁰ is a water solubilizing group.

18. The method of any one of claims 11-17, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

19. The method of any one of claims 11-18, wherein R⁸, R⁹ and R¹⁰ are independently hydrogen, CH₃, C(O)CH₃, or PO₃H₂.

20. The method of any one of claims 11-19, wherein R⁸, R⁹ and R¹⁰ are independently hydrogen, CH₃, or C(O)CH₃.

21. The method of any one of claims 11-20, wherein the compound is of Formula VI-B: 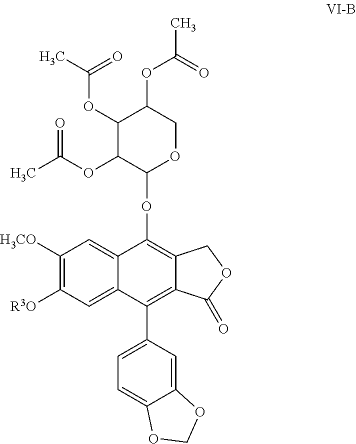
wherein
R³ is hydrogen, halogen, substituted or unsubstituted C₁-C₄ alkyl substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

22. The method of claim 21, wherein R³ is a water solubilizing group.

23. The method of any one of claims 21-22, wherein R³ is hydrogen, substituted or unsubstituted C₁-C₄ alkyl, or substituted or unsubstituted phosphonyl.

24. The method of any one of claims 21-23, wherein R³ is hydrogen, CH₃, or PO₃H₂.

25. The method of any one of claims 21-24, wherein the compound is of Formula VI-B-1: 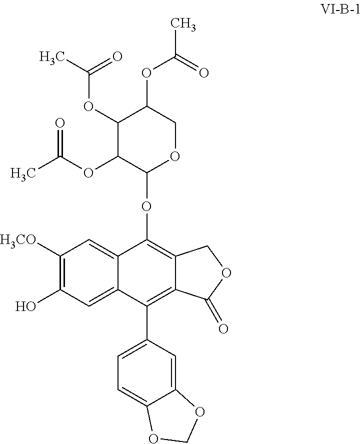 [SMILES: COc1cc2c(OC3OCC(OC(C)=O)C(OC(C)=O)C3OC(C)=O)c3c(c(-c4ccc5c(c4)OCO5)c2cc1O)C(=O)OC3]
or a pharmaceutically acceptable salt or prodrug thereof.

26. The method of any one of claims 21-24, wherein the compound is of Formula VI-B-2: 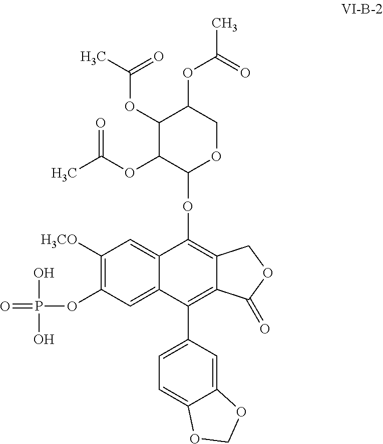 [SMILES: COc1cc2c(OC3OCC(OC(C)=O)C(OC(C)=O)C3OC(C)=O)c3c(c(-c4ccc5c(c4)OCO5)c2cc1OP(=O)(O)O)C(=O)OC3]
or a pharmaceutically acceptable salt or prodrug thereof.

27. The method of any one of claims 11-20, wherein the compound is of Formula VI-C: 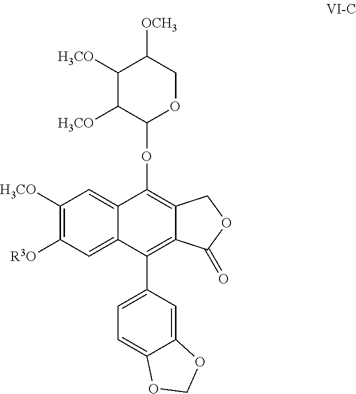
wherein
R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof

28. The method of claim 27, wherein R³ is a water solubilizing group.

29. The method of any one of claims 27-28, wherein R³ is hydrogen, substituted or unsubstituted C₁-C₄ alkyl, or substituted or unsubstituted phosphonyl.

30. The method of any one of claims 27-29, wherein R³ is hydrogen, CH₃, or PO₃H₂.

31. The method of any one of claims 27-30, wherein the compound is of Formula VI-C-1: 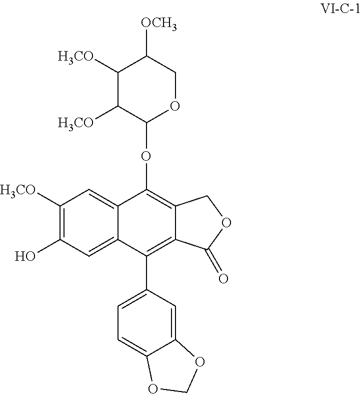 [SMILES: COc1cc2c(OC3OCC(OC)C(OC)C3OC)c3c(c(-c4ccc5c(c4)OCO5)c2cc1O)C(=O)OC3]
or a pharmaceutically acceptable salt or prodrug thereof.

32. The method of any one of claims 27-30, wherein the compound is of Formula VI-C-2: 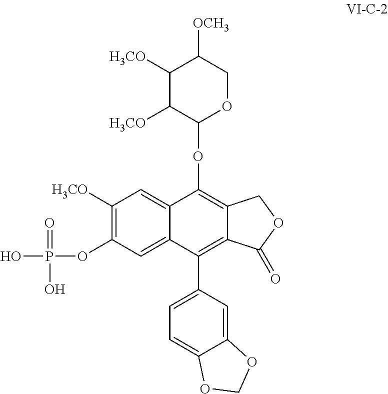 [SMILES: COc1cc2c(OC3OCC(OC)C(OC)C3OC)c3c(c(-c4ccc5c(c4)OCO5)c2cc1OP(=O)(O)O)C(=O)OC3]
or a pharmaceutically acceptable salt or prodrug thereof.

33. The method of any one of claims 1-10, wherein the compound is of Formula VI-D: 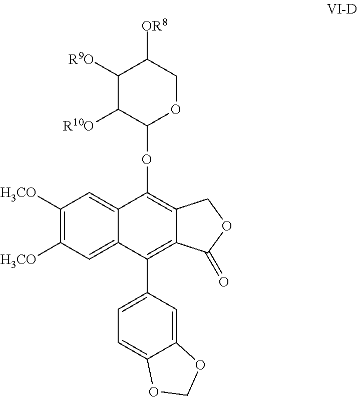
wherein
R⁵, R⁹ and R¹⁰ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfo substituted or unsubstituted sulfonamide, or substituted or unsubstituted thio;
or a pharmaceutically acceptable salt or prodrug thereof.

34. The method of claim 33, wherein R⁸ is a water solubilizing group.

35. The method of any one of claims 33-34, wherein R⁹ is a water solubilizing group,

36. The method of any one of claims 33-35, wherein R⁸ and R⁹ are independently hydrogen, substituted or unsubstituted C₁-C₆ alkyl, substituted or unsubstituted C₁-C₆ acyl, or substituted or unsubstituted phosphonyl.

37. The method of any one of claims 33-36, wherein R⁸ and R⁹ are independently hydrogen, CH₃, C(O)CH₃, or PO₃H₂.

38. The method of any one of claims 33-37, wherein R⁸ and R⁹ are independently hydrogen, CH₃, or C(O)CH₃.

39. The method of any one of claims 33-38, wherein R¹⁰ is a water solubilizing group.

40. The method of any one of claims 33-39, wherein R¹⁰ is hydrogen, substituted or unsubstituted C₁-C₆ alkyl, substituted or unsubstituted C₁-C₆ acyl, or substituted or unsubstituted phosphonyl.

41. The method of any one of claims 33-40, wherein R¹⁰ is hydrogen, CH₃, C(O)CH₃, or PO₃H₂.

42. The method of any one of claims 33-41, wherein the compound is of Formula VI-E: 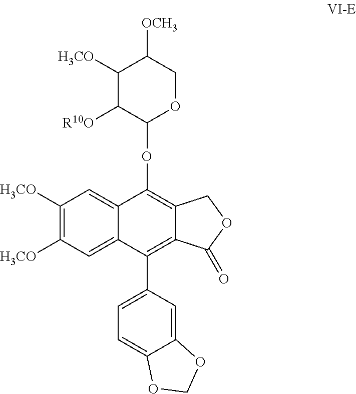
wherein
R¹⁰ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

43. The method of claim 42, wherein R¹⁰ is a water solubilizing group.

44. The method of any one of claims 42-43, wherein R¹⁰ is hydrogen, substituted or unsubstituted C₁-C₆ alkyl, substituted or unsubstituted C₁-C₆ acyl, or substituted or unsubstituted phosphonyl.

45. The method of any one of claims 42-44, wherein R¹⁰ is hydrogen, CH₃, C(O)CH₃, or PO₃H₂.

46. The method of any one of claims 42-45, wherein R¹⁰ is hydrogen or PO₃H₂.

47. The method of any one of claims 42-46, wherein the compound is of Formula VI-E-1: 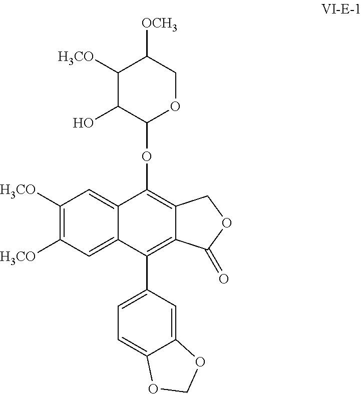 [SMILES: COc1cc2c(OC3OCC(OC)C(OC)C3O)c3c(c(-c4ccc5c(c4)OCO5)c2cc1OC)C(=O)OC3]
or a pharmaceutically acceptable salt or prodrug thereof.

48. The method of any one of claims 42-46, wherein the compound is of Formula VI-E-2: 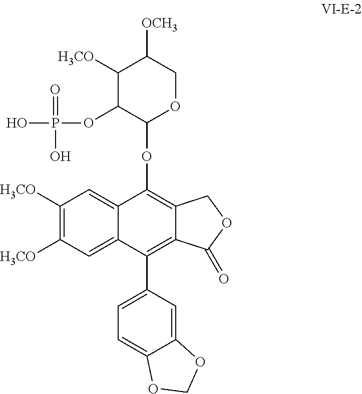 [SMILES: COc1cc2c(OC3OCC(OC)C(OC)C3OP(=O)(O)O)c3c(c(-c4ccc5c(c4)OCO5)c2cc1OC)C(=O)OC3]
or a pharmaceutically acceptable salt or prodrug thereof.

49. The method of any one of claims 33-41, wherein the compound is of Formula VI-F: 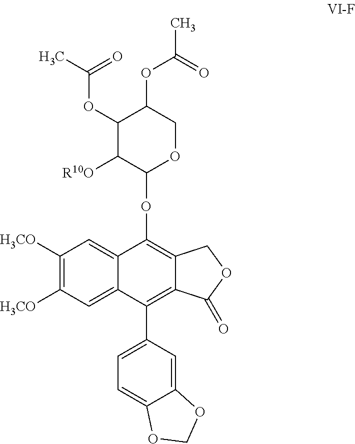
wherein
R¹⁰ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

50. The method of claim 49, wherein R¹⁰ is a water solubilizing group.

51. The method of any one of claims 49-50, wherein R¹⁰ is hydrogen, substituted or unsubstituted C₁-C₆ alkyl, substituted or unsubstituted C₁-C₆ acyl, or substituted or unsubstituted phosphonyl.

52. The method of any one of claims 49-51, wherein R¹⁰ is hydrogen, C(O)CH₃, or PO₃H₂.

53. The method of any one of claims 49-52, wherein R¹⁰ is hydrogen or PO₃H₂.

54. The method of any one of claims 49-53, wherein the compound is of Formula VI-F-1: 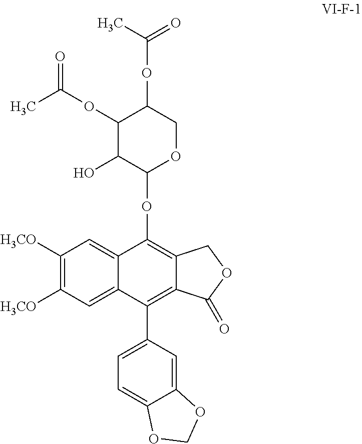 [SMILES: COc1cc2c(OC3OCC(OC(C)=O)C(OC(C)=O)C3O)c3c(c(-c4ccc5c(c4)OCO5)c2cc1OC)C(=O)OC3]
or a pharmaceutically acceptable salt or prodrug thereof.

55. The method of any one of claims 33-41, wherein the compound is of Formula VI-D-1: 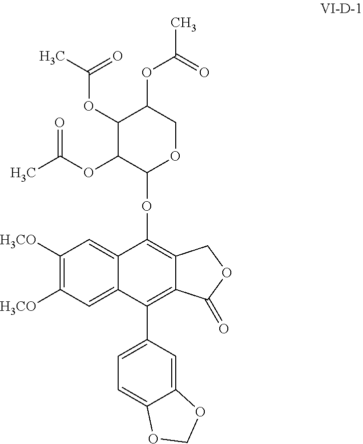 [SMILES: COc1cc2c(OC3OCC(OC(C)=O)C(OC(C)=O)C3OC(C)=O)c3c(c(-c4ccc5c(c4)OCO5)c2cc1OC)C(=O)OC3]
or a pharmaceutically acceptable salt or prodrug thereof.

56. The method of any one of claims 33-41, wherein the compound is of Formula VI-D-2: 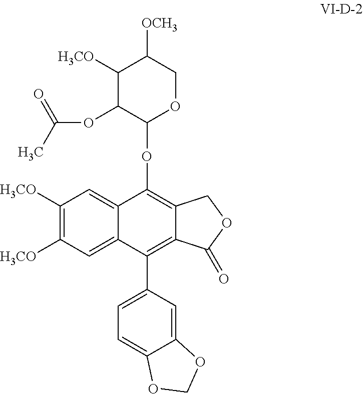 [SMILES: COc1cc2c(OC3OCC(OC)C(OC)C3OC(C)=O)c3c(c(-c4ccc5c(c4)OCO5)c2cc1OC)C(=O)OC3]
or a pharmaceutically acceptable sa t or prodrug thereof.

57. The method of any one of claims 33-41, wherein the compound is of Formula VI-D-3: 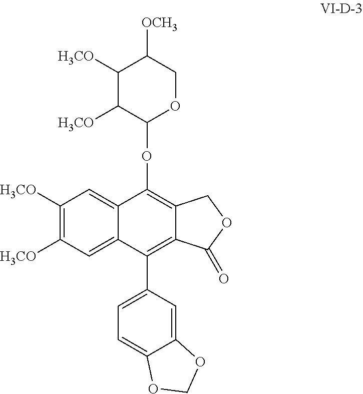 [SMILES: COc1cc2c(OC3OCC(OC)C(OC)C3OC)c3c(c(-c4ccc5c(c4)OCO5)c2cc1OC)C(=O)OC3]
or a pharmaceutically acceptable salt or prodrug thereof.

58. The method of any one of claims 33-41, wherein the compound is of Formula VI-D-4: 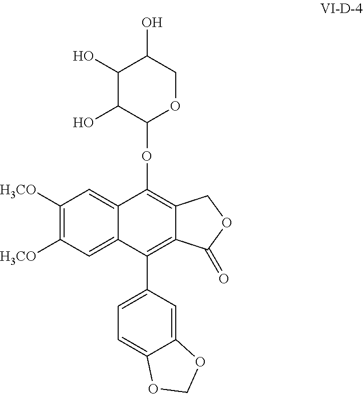 [SMILES: COc1cc2c(OC3OCC(O)C(O)C3O)c3c(c(-c4ccc5c(c4)OCO5)c2cc1OC)C(=O)OC3]
or a pharmaceutically acceptable salt or prodrug thereof.

59. The method of any one of claims 33-41, wherein the compound is of Formula VI-D-5: 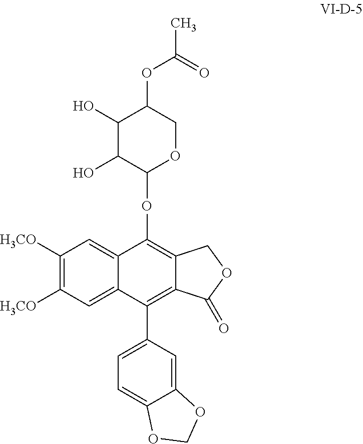 [SMILES: COc1cc2c(OC3OCC(OC(C)=O)C(O)C3O)c3c(c(-c4ccc5c(c4)OCO5)c2cc1OC)C(=O)OC3]
or a pharmaceutically acceptable salt or prodrug thereof.

60. The method of any one of claims 33-41, wherein the compound is of Formula VI-D-6: 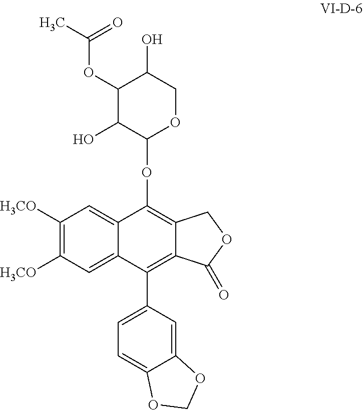 [SMILES: COc1cc2c(OC3OCC(O)C(OC(C)=O)C3O)c3c(c(-c4ccc5c(c4)OCO5)c2cc1OC)C(=O)OC3]
or a pharmaceutically acceptable salt or prodrug thereof.

61. The method of any one of claims 1-60, wherein the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

62. The method of any one of claims 1-61, wherein the cancer is colon cancer.

63. The method of any one of claims 1-62, further comprising administering a second compound or composition, wherein the second compound or composition includes an anticancer agent.

64. The method of any one of claims 1-63, further comprising administering an effective amount of ionizing radiation to the subject.

65. A method of killing a tumor cell in a subject, comprising: contacting the tumor cell with an effective amount of a composition comprising a compound of Formula VI: 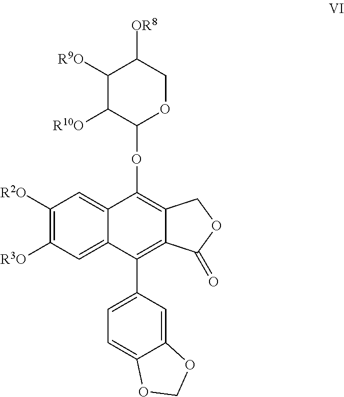
wherein
R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio; or R² and R³ taken together with the atoms to which they are attached form a substituted or unsubstituted 5 to 7 membered heterocyclic moiety;
R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

66. The method of claim 65, wherein R² is a water solubilizing group.

67. The method of any one of claims 65-66, wherein R³ is a water solubilizing group.

68. The method of any one of claims 65-67, wherein R² and R³ are independently hydrogen, substituted or unsubstituted C₁-C₄ alkyl, or substituted or unsubstituted phosphonyl.

69. The method of any one of claims 65-68, wherein R² and R³ are independently hydrogen, CH₃, or PO₃H₂.

70. The method of any one of claims 65-69, wherein R⁸ is a water solubilizing group.

71. The method of any one of claims 65-70, wherein R⁹ is a water solubilizing group.

72. The method of any one of claims 65-71, wherein R¹⁰ is a water group.

73. The method of any one of claims 65-72, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

74. The method of any one of claims 65-73, wherein R⁸, R⁹ and R¹⁰ are independently hydrogen, CH₃, C(O)CH₃, or PO₃H₂.

75. The method of any one of claims 65-74, wherein the compound is of Formula VI-A: 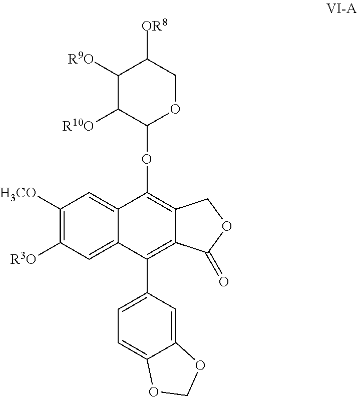
wherein
R³ is hydrogen, halogen, substituted or unsubstituted alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
R⁹, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

76. The method of claim 75, wherein R³ is a water solubilizing group.

77. The method of any one of claims 75-76, wherein R³ is hydrogen, substituted or unsubstituted C₁-C₄ alkyl, or substituted or unsubstituted phosphonyl.

78. The method of any one of claims 75-77, wherein R³ is hydrogen, CH₃, or PO₃H₂.

79. The method of any one of claims 75-78, wherein R⁸ is a water solubilizing group.

80. The method of any one of claims 75-79, wherein R⁹ is a water solubilizing group.

81. The method of any one of claims 75-80, wherein R¹⁰ is a water solubilizing group.

82. The method of any one of claims 75-81, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

83. The method of any one of claims 75-82, wherein R⁸, R⁹ and R¹⁰ are independently hydrogen, CH₃, C(O)CH₃, or PO₃H₂.

84. The method of any one of claims 75-83, wherein R⁸, R⁹ and R¹⁰ are independently hydrogen, CH₃, or C(O)CH₃.

85. The method of any one of claims 75-84, wherein the compound is of Formula VI-B: 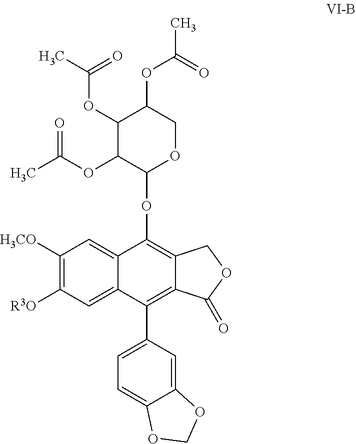
wherein
R³ is hydrogen, halogen, substituted or unsubstituted C₁-C₄ alkyl substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

86. The method of claim 85, wherein R³ is a water solubilizing group.

87. The method of any one of claims 85-86, wherein R³ is hydrogen, substituted or unsubstituted C₁-C₄ alkyl, or substituted or unsubstituted phosphonyl.

88. The method of any one of claims 85-87, wherein R³ is hydrogen, CH₃, or PO₃H₂.

89. The method of any one of claims 85-88, wherein the compound is of Formula VI-B-1: 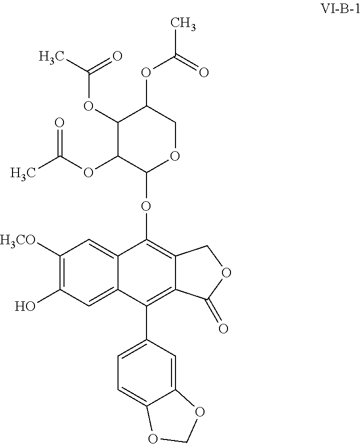 [SMILES: COc1cc2c(OC3OCC(OC(C)=O)C(OC(C)=O)C3OC(C)=O)c3c(c(-c4ccc5c(c4)OCO5)c2cc1O)C(=O)OC3]
or a pharmaceutically acceptable salt or prodrug thereof.

90. The method of any one of claims 85-88, wherein the compound is of Formula VI-B-2: 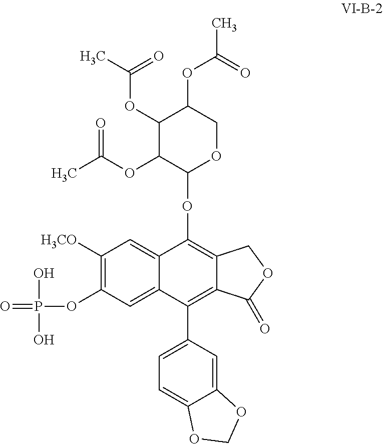 [SMILES: COc1cc2c(OC3OCC(OC(C)=O)C(OC(C)=O)C3OC(C)=O)c3c(c(-c4ccc5c(c4)OCO5)c2cc1OP(=O)(O)O)C(=O)OC3]
or a pharmaceutically acceptable salt or prodrug thereof.

91. The method of any one of claims 74-84, wherein the compound is of Formula VI-C: 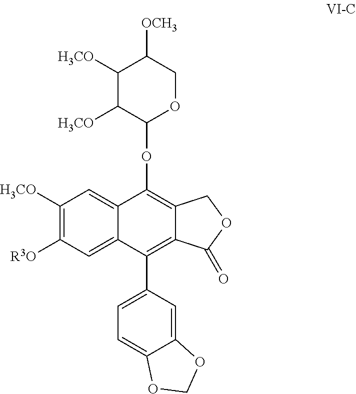
wherein
R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof

92. The method of claim 91, wherein R³ is a water solubilizing group.

93. The method of any one of claims 91-92, wherein R³ is hydrogen, substituted or unsubstituted C₁-C₄ alkyl, or substituted or unsubstituted phosphonyl.

94. The method of any one of claims 91-93, wherein R³ is hydrogen, CH₃, or PO₃H₂.

95. The method of any one of claims 91-94, wherein the compound is of Formula VI-C-1: 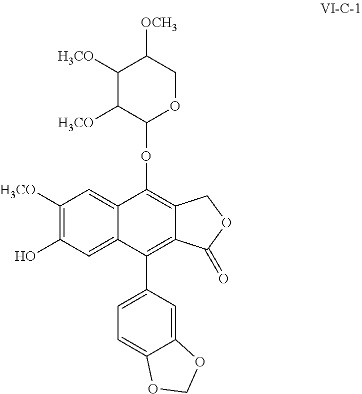 [SMILES: COc1cc2c(OC3OCC(OC)C(OC)C3OC)c3c(c(-c4ccc5c(c4)OCO5)c2cc1O)C(=O)OC3]
or a pharmaceutically acceptable salt or prodrug thereof.

96. The method of any one of claims 91-94, wherein the compound is of Formula VI-C-2: 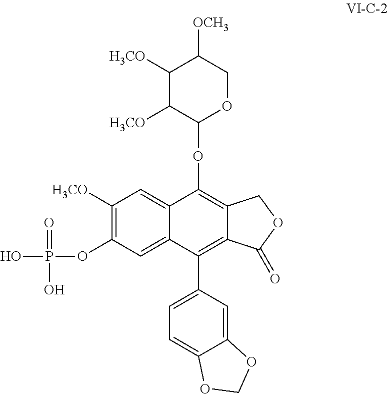 [SMILES: COc1cc2c(OC3OCC(OC)C(OC)C3OC)c3c(c(-c4ccc5c(c4)OCO5)c2cc1OP(=O)(O)O)C(=O)OC3]
or a pharmaceutically acceptable salt or prodrug thereof.

97. The method of any one of claims 65-74, wherein the compound is of Formula VI-D: 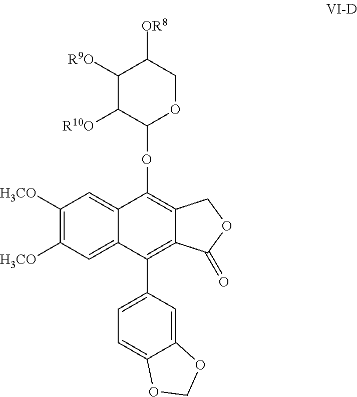
wherein
R⁸, R⁹ and R¹⁰ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfo substituted or unsubstituted sulfonamide, or substituted or unsubstituted thio;
or a pharmaceutically acceptable salt or prodrug thereof.

98. The method of claim 97, wherein R⁸ is a water solubilizing group.

99. The method of any one of claims 97-98, wherein R⁹ is a water solubilizing group.

100. The method of any one of claims 97-99, wherein R⁸ and R⁹ are independently hydrogen, substituted or unsubstituted C₁-C₆ alkyl, substituted or unsubstituted C₁-C₆ acyl, or substituted or unsubstituted phosphonyl.

101. The method of any one of claims 97-100, wherein R⁸ and R⁸ are independently hydrogen, CH₃, C(O)CH₃, or PO₃H₂.

102. The method of any one of claims 97-101, wherein R⁸ and R⁹ are independently hydrogen, CH₃, or C(O)CH₃.

103. The method of any one of claims 97-102, wherein R¹⁰ is a water solubilizing group.

104. The method of any one of claims 97-103, wherein R¹⁰ is hydrogen, substituted or unsubstituted C₁-C₆ alkyl, substituted or unsubstituted C₁-C₆ acyl, or substituted or unsubstituted phosphonyl.

105. The method of any one of claims 97-104, wherein R¹⁰ is hydrogen, CH₃, C(O)CH₃, or PO₃H₂.

106. The method of any one of claims 97-105, wherein the compound is of Formula VI-E: 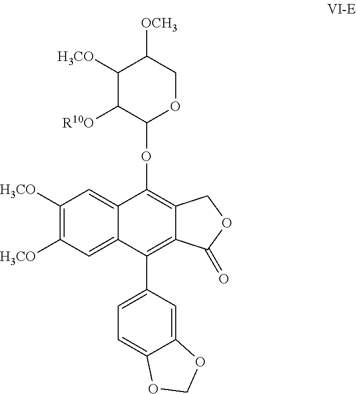
wherein
R¹⁰ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

107. The method of claim 106, wherein R¹⁰ is a water solubilizing group.

108. The method of any one of claims 106-107, wherein R¹⁰ is hydrogen, substituted or unsubstituted C₁-C₆ alkyl, substituted or unsubstituted C₁-C₆ acyl, or substituted or unsubstituted phosphonyl.

109. The method of any one of claims 106-108, wherein R¹⁰ is hydrogen, CH₃, C(O)CH₃, or PO₃H₂.

110. The method of any one of claims 106-109, wherein R¹⁰ is hydrogen or PO₃H₂.

111. The method of any one of claims 106-110, wherein the compound is of Formula VI-E-1 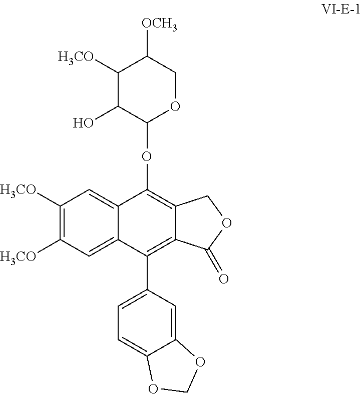 [SMILES: COc1cc2c(OC3OCC(OC)C(OC)C3O)c3c(c(-c4ccc5c(c4)OCO5)c2cc1OC)C(=O)OC3]
or a pharmaceutically acceptable salt or prodrug thereof.

112. The method of any one of claims 106-110, wherein the compound is of Formula VI-E-2: 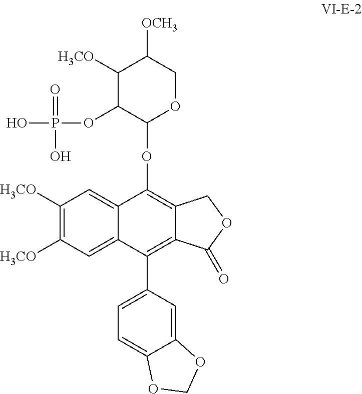 [SMILES: COc1cc2c(OC3OCC(OC)C(OC)C3OP(=O)(O)O)c3c(c(-c4ccc5c(c4)OCO5)c2cc1OC)C(=O)OC3]
or a pharmaceutically acceptable salt or prodrug thereof.

113. The method of any one of claims 97-105, wherein the compound is of Formula VI-F: 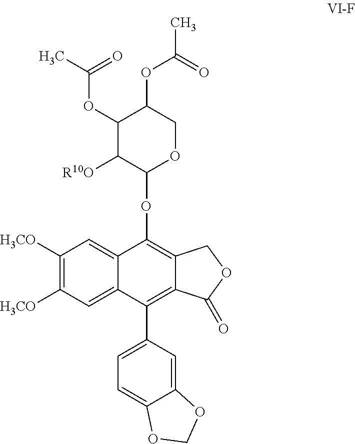
wherein
R¹⁰ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof.

114. The method of claim 113, wherein R¹⁰ is a water solubilizing group.

115. The method of any one of claims 113-114, wherein R¹⁰ is hydrogen, substituted or unsubstituted C₁-C₆ alkyl, substituted or unsubstituted C₁-C₆ acyl, or substituted or unsubstituted phosphonyl.

116. The method of any one of claims 113-115, wherein R¹⁰ is hydrogen, CH₃, C(O)CH₃, or PO₃H₂.

117. The method of any one of claims 113-116, wherein R¹⁰ is hydrogen or PO₃H₂.

118. The method of any one of claims 113-117, wherein the compound is of Formula VI-F-1; 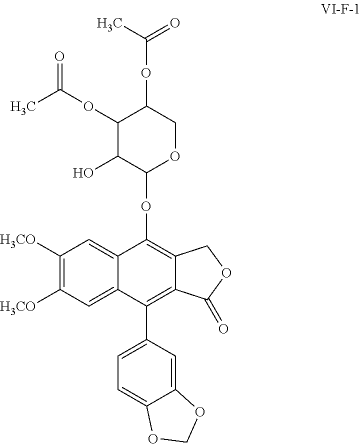 [SMILES: COc1cc2c(OC3OCC(OC(C)=O)C(OC(C)=O)C3O)c3c(c(-c4ccc5c(c4)OCO5)c2cc1OC)C(=O)OC3]
or a pharmaceutically acceptable salt or prodrug thereof.

119. The method of any one of claims 97-105, wherein the compound is of Formula VI-D-1: 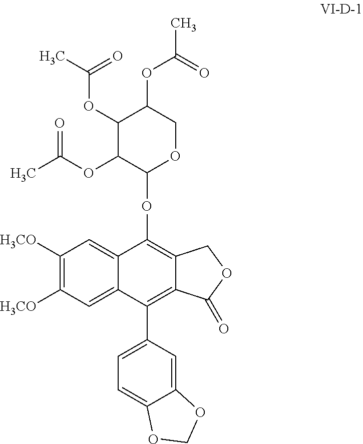 [SMILES: COc1cc2c(OC3OCC(OC(C)=O)C(OC(C)=O)C3OC(C)=O)c3c(c(-c4ccc5c(c4)OCO5)c2cc1OC)C(=O)OC3]
or a pharmaceutically acceptable salt or prodrug thereof.

120. The method of any one of claims 97-105, wherein the compound is of Formula VI-D-2: 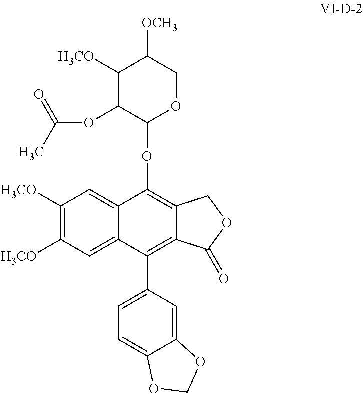 [SMILES: COc1cc2c(OC3OCC(OC)C(OC)C3OC(C)=O)c3c(c(-c4ccc5c(c4)OCO5)c2cc1OC)C(=O)OC3]
or a pharmaceutically acceptable salt or prodrug thereof.

121. The method of any one of claims 97-105, wherein the compound is of Formula VI-D-3: 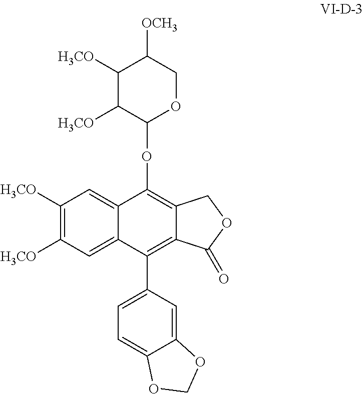 [SMILES: COc1cc2c(OC3OCC(OC)C(OC)C3OC)c3c(c(-c4ccc5c(c4)OCO5)c2cc1OC)C(=O)OC3]
or a pharmaceutically acceptable salt or prodrug thereof.

122. The method of any one of claims 97-105, wherein the compound is of Formula VI-D-4: 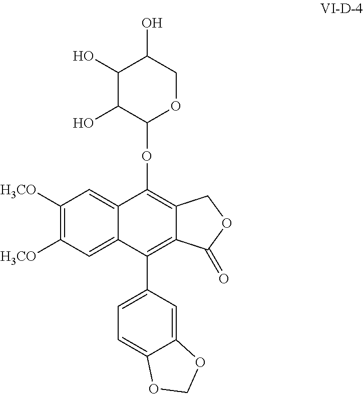 [SMILES: COc1cc2c(OC3OCC(O)C(O)C3O)c3c(c(-c4ccc5c(c4)OCO5)c2cc1OC)C(=O)OC3]
or a pharmaceutically acceptable salt or prodrug thereof.

123. The method of any one of claims 97-105, wherein the compound is of Formula VI-D-5: 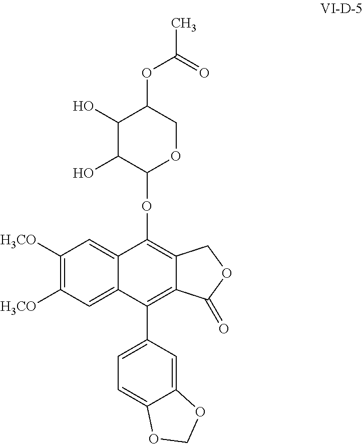 [SMILES: COc1cc2c(OC3OCC(OC(C)=O)C(O)C3O)c3c(c(-c4ccc5c(c4)OCO5)c2cc1OC)C(=O)OC3]
or a pharmaceutically acceptable salt or prodrug thereof.

124. The method of any one of claims 97-105, wherein the compound is of Formula VI-D-6: 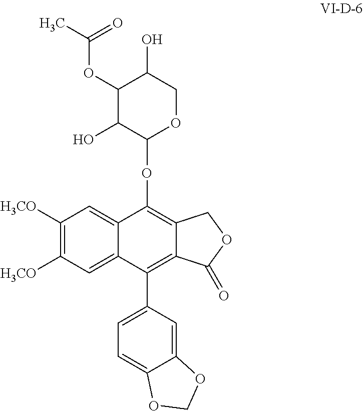 [SMILES: COc1cc2c(OC3OCC(O)C(OC(C)=O)C3O)c3c(c(-c4ccc5c(c4)OCO5)c2cc1OC)C(=O)OC3]
or a pharmaceutically acceptable salt or prodrug thereof.

125. The method of any one of claims 65-124, further comprising contacting the tumor cell with a second compound or composition, wherein the second compound or composition includes an anticancer agent,

126. The method of any one of claims 65-125, wherein the tumor cell is a colon cancer cell

127. The method of any of claims 65-126, further comprising irradiating the tumor cell with an effective amount of ionizing radiation.

128. A composition, comprising a compound of Formula II-B: 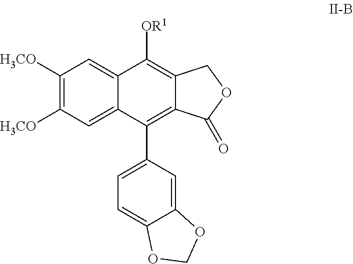
wherein
R¹ is hydrogen, halogen, formyl, substituted or unsubstituted C₁-C₆ alkyl, substituted or unsubstituted C₄-C₁₀ cycloalkyl, substituted or unsubstituted C₄-C₁₀ heterocycloalkyl, substituted or unsubstituted alkoxycarbonyl, hydroxycarbonyl, substituted or unsubstituted acyl; or a pharmaceutically acceptable salt or prodrug thereof; and
a pharmaceutically acceptable carrier.

129. The composition of claim 128, wherein R¹ is selected from:
H,

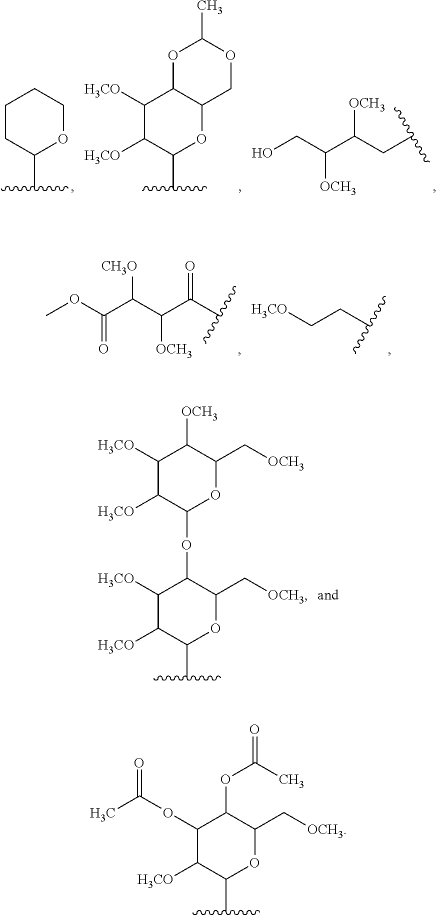

130. The composition of any one of claims 128-129, wherein the compound is of Formula II-B-1: 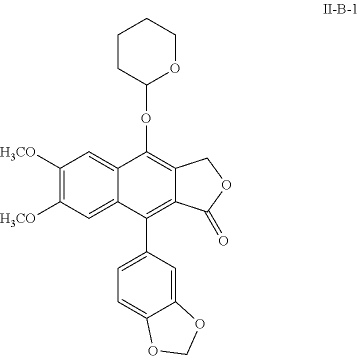 [SMILES: COc1cc2c(OC3CCCCO3)c3c(c(-c4ccc5c(c4)OCO5)c2cc1OC)C(=O)OC3]
or a pharmaceutically acceptable salt or prodrug thereof.

131. The composition of any one of claims 128-129, wherein the compound is of Formula II-B-2: 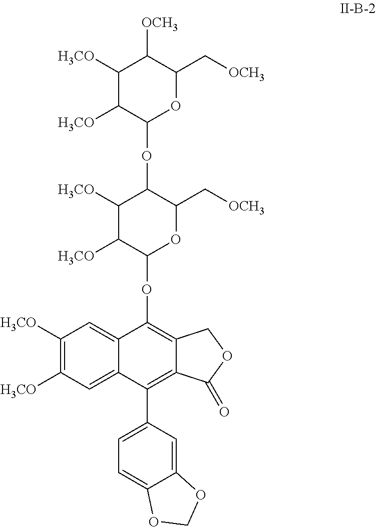 [SMILES: COCC1OC(OC2C(COC)OC(Oc3c4c(c(-c5ccc6c(c5)OCO6)c5cc(OC)c(OC)cc35)C(=O)OC4)C(OC)C2OC)C(OC)C(OC)C1OC]
or a pharmaceutically acceptable salt or prodrug thereof.

132. The composition of any one of claims 128-129, wherein the compound is of Formula II-B-3: 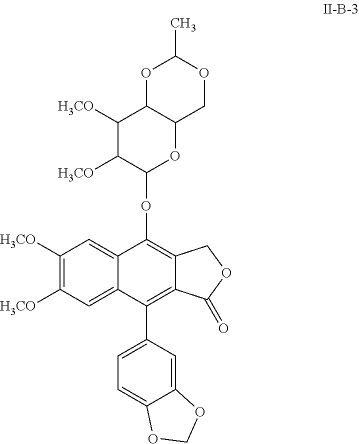 [SMILES: COc1cc2c(OC3OC4COC(C)OC4C(OC)C3OC)c3c(c(-c4ccc5c(c4)OCO5)c2cc1OC)C(=O)OC3]
or a pharmaceutically acceptable salt or prodrug thereof.

133. The composition of any one of claims 128-129, wherein the compound is of Formula II-B-4: 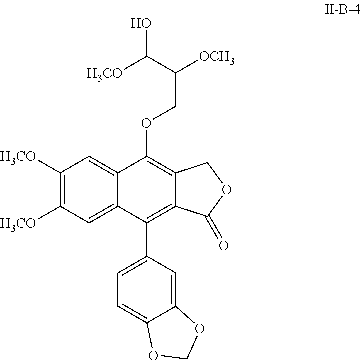 [SMILES: COc1cc2c(OCC(OC)C(O)OC)c3c(c(-c4ccc5c(c4)OCO5)c2cc1OC)C(=O)OC3]
or a pharmaceutically acceptable salt or prodrug thereof.

134. The composition of any one of claims 128-129, wherein the compound is of Formula II-B-5: 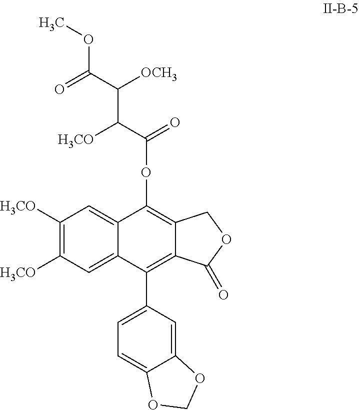 [SMILES: COC(=O)C(OC)C(OC)C(=O)Oc1c2c(c(-c3ccc4c(c3)OCO4)c3cc(OC)c(OC)cc13)C(=O)OC2]
or a pharmaceutically acceptable salt or product thereof.

135. The composition of any one of claims 128-129, wherein the compound is of Formula II-B-6: 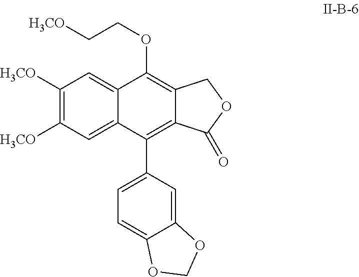 [SMILES: COCCOc1c2c(c(-c3ccc4c(c3)OCO4)c3cc(OC)c(OC)cc13)C(=O)OC2]
or a pharmaceutically acceptable salt or prodrug thereof.

136. The composition of any one of claims 128-129, wherein the compound is of Formula II-B-7: 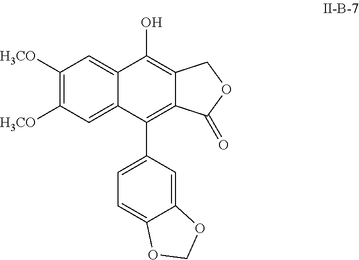 [SMILES: COc1cc2c(O)c3c(c(-c4ccc5c(c4)OCO5)c2cc1OC)C(=O)OC3]
or a pharmaceutically acceptable salt or prodrug thereof.

137. A composition, comprising a compound of Formula IV: 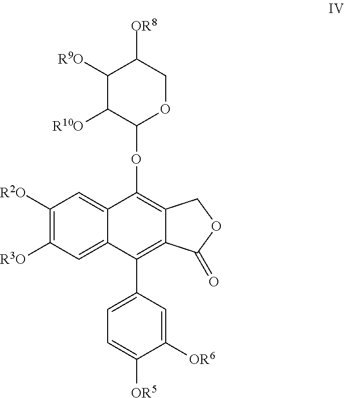
wherein
R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
R⁵ and R⁶ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R⁵ and R⁶ taken together with the atoms to which they are attached form a substituted or unsubstituted 5 to 7 membered heterocyclic moiety;
R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl substituted or unsubstituted sulfonyl, substituted or unsubstituted sulfonamide, or substituted or unsubstituted thio;
with the proviso that at least one of R²-R¹⁰ is a substituted or unsubstituted phosphonyl; or a pharmaceutically acceptable salt or prodrug thereof; and
a pharmaceutically acceptable carrier.

138. The composition of claim 137, wherein R² is a water solubilizing group.

139. The composition of any one of claim 137-138, wherein R³ is a water solubilizing group.

140. The composition of any one of claims 137-139, wherein R² and R³ are independently hydrogen, substituted or unsubstituted C₁-C₄ alkyl, or substituted or unsubstituted phosphonyl.

141. The composition of any one of claims 137-140, wherein R² and R³ are independently hydrogen, CH₃, or PO₃H₂.

142. The composition of any one of claims 137-141, wherein R⁵ is a water solubilizing group.

143. The composition of any one of claims 137-142, wherein R⁶ is a water solubilizing group.

144. The composition of any one of claims 137-143, wherein R⁵ and R⁶ are independently hydrogen, substituted or unsubstituted C₁-C₄ alkyl, substituted or unsubstituted phosphonyl, or together with the atoms to which they are attached form a 5 membered heterocyclic group.

145. The composition of any one of claims 137-144, wherein R⁵ and R⁶ are independently hydrogen, CH₃, PO₃H₂, or together with the atoms to which they are attached form a 5 membered heterocyclic group.

146. The composition of any one of claims 137-145, wherein R² is CH₃.

147. The composition of any one of claims 137-146, wherein R³ is CH₃.

148. The composition of any one of claims 137-147, wherein R⁶ is CH₃.

149. The composition of any one of claims 137-148, wherein R⁸ is a water solubilizing group.

150. The composition of any one of claims 137-149, wherein R⁹ is a water solubilizing group.

151. The composition of any one of claims 137-150, wherein R¹⁰ is a water solubilizing group.

152. The composition of any one of claims 137-151, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

153. The composition of any one of claims 137-152, wherein R⁸, R⁹ and R¹⁰ are independently hydrogen, CH₃, C(O)CH₃, or PO₃H₂.

154. The composition of any one of claims 137-153, wherein R⁸, R⁹ and R¹⁰ are independently hydrogen, CH₃, or C(O)CH₃.

155. The composition of any one of claims 137-154, wherein the compound is of Formula IV-A: 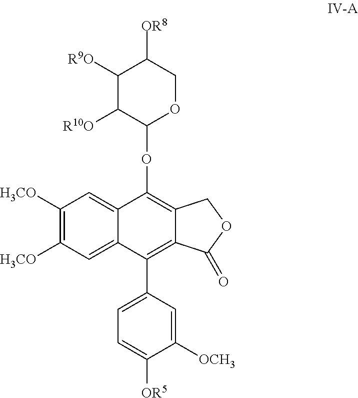
wherein
R⁵ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl substituted or unsubstituted sulfonyl, substituted or unsubstituted sulfonamide, or substituted or unsubstituted thio;
with the proviso that at least one of R⁵, R⁸, R⁹ or R¹⁰ is a substituted or unsubstituted phosphonyl;
or a pharmaceutically acceptable salt or prodrug thereof.

156. The composition of claim 155, wherein R⁵ is a water solubilizing group.

157. The composition of any one of claims 155-156, wherein R⁵ is hydrogen, substituted or unsubstituted C₁-C₄ alkyl, or substituted or unsubstituted phosphonyl.

158. The composition of any one of claims 155-157, wherein R⁵ is hydrogen, CH₃, or PO₃H₂.

159. The composition of any one of claims 155-158, wherein R⁸ is a water solubilizing group.

160. The composition of any one of claims 155-159, wherein R⁹ is a water solubilizing group.

161. The composition of any one of claims 155-160, wherein R¹⁰ is a water solubilizing group.

162. The composition of any one of claims 155-161, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl,

163. The composition of any one of claims 155-162, wherein R⁸, R⁹ and R¹⁰ are independently hydrogen, CH₃, C(O)CH₃, or PO₃H₂.

164. The composition of any one of claims 155-163, wherein R⁸, R⁹ and R¹⁰ are independently hydrogen, CH₃, or C(O)CH₃.

165. The composition of any one of claims 155-164, wherein the compound is of Formula IV-B: 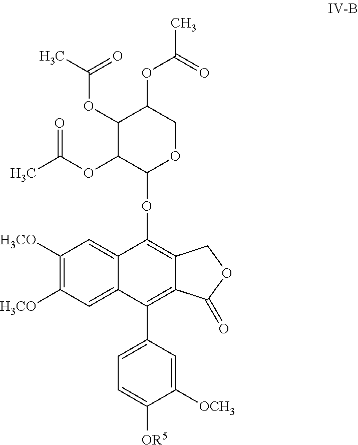
wherein
R⁵ is a water solubilizing group;
or a pharmaceutically acceptable salt or prodrug thereof.

166. The composition of claim 165, wherein R⁵ is a substituted or unsubstituted phosphonyl.

167. The composition of any one of claims 165-166, wherein the compound is of Formula IV-B-2: 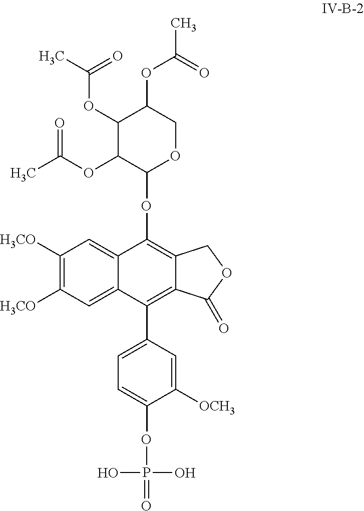 [SMILES: COc1cc2c(OC3OCC(OC(C)=O)C(OC(C)=O)C3OC(C)=O)c3c(c(-c4ccc(OP(=O)(O)O)c(OC)c4)c2cc1OC)C(=O)OC3]
or a pharmaceutically acceptable salt or prodrug thereof.

168. The composition of any one of claims 155-164, wherein the compound is of Formula IV-C: 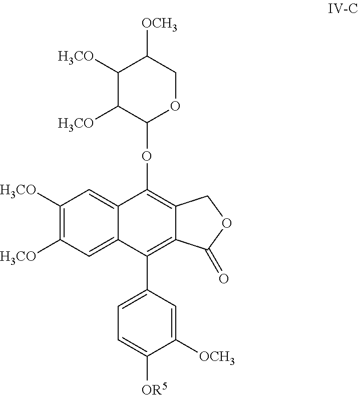
wherein
R⁵ is a water solubilizing group;
or a pharmaceutically acceptable salt or prodrug thereof.

169. The composition of claim 168, wherein R⁵ is a substituted or unsubstituted phosphonyl.

170. The composition of any one of claims 168-169, wherein the compound is of Formula IV-C-2: 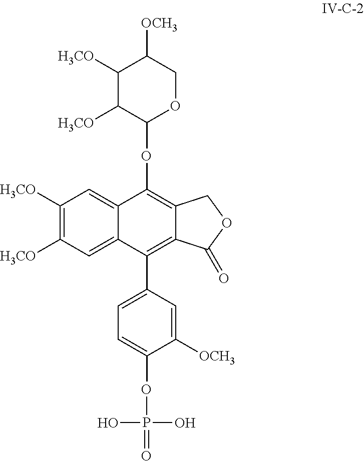 [SMILES: COc1cc2c(OC3OCC(OC)C(OC)C3OC)c3c(c(-c4ccc(OP(=O)(O)O)c(OC)c4)c2cc1OC)C(=O)OC3]
or a pharmaceutically acceptable salt or prodrug thereof.

171. A composition, comprising a compound of Formula VI: 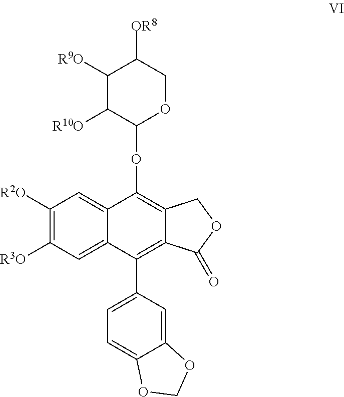
wherein
R² and R³ are independently hydrogen, halogen, substituted or unsubstituted C₁-C₄ alkyl substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio; or R² and R³ taken together with the atoms to which they are attached form a substituted or unsubstituted 5 to 7 membered heterocyclic moiety;
R⁸, R⁹ and R¹⁰ are independently hydrogen, halogen, substituted or unsubstituted C₁-C₆ alkyl., substituted or unsubstituted C₁-C₆ alkoxycarbonyl, hydroxycarbonyl, substituted, unsubstituted C₁-C₆ acyl, substituted or unsubstituted amino, substituted or unsubstituted amido, substituted or unsubstituted C₁-C₆carbamoyl, substituted or unsubstituted phosphonyl, substituted or unsubstituted sulfinyl, substituted or unsubstituted sulfo substituted or unsubstituted sulfonamide, or substituted or unsubstituted thio;
with the proviso that at least one of R², R³, R⁸, R⁹, or R¹⁰ is a substituted or unsubstituted phosphonyl;
or a pharmaceutically acceptable salt or prodrug thereof; and
a pharmaceutically acceptable carrier.

172. The composition of claim 171, wherein R² is a water solubilizing group.

173. The composition of any one of claims 171-172, wherein R³ is a water solubilizing group.

174. The composition of any one of claims 171-173, wherein R² and R³ are independently hydrogen, substituted or unsubstituted C₁-C₄ alkyl, or substituted or unsubstituted phosphonyl.

175. The composition of any one of claims 171-174, wherein R² and R³ are independently hydrogen, CH₃, or PO₃H₂.

176. The composition of any one of claims 171-175, wherein R⁸ is a water solubilizing group.

177. The composition of any one of claims 171-176, wherein R⁹ is a water solubilizing group.

178. The composition o any one of claims 171-177, wherein R¹⁰ is a water solubilizing group.

179. The composition of any one of claims 171-178, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

180. The composition of any one of claims 171-179, wherein R⁸, R⁹ and R¹⁰ are independently hydrogen, CH₃, C(O)CH₃, or PO₃H₂.

181. The composition of any one of claims 171-180, wherein the compound is of Formula VI-A: 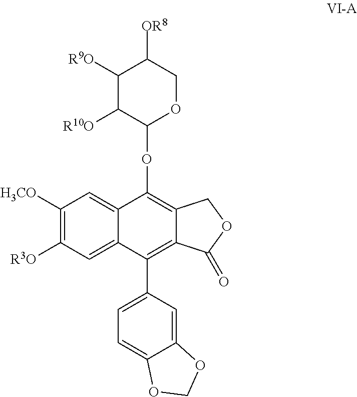
wherein
R³ is hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
R⁸, R⁹ and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
with the proviso that at least one of R³, R⁸, R⁹, or R¹⁰ is a substituted or unsubstituted phosphonyl;
or a pharmaceutically acceptable salt or prodrug thereof.

182. The composition of claim 181, wherein R³ is a water solubilizing group.

183. The composition of any one of claims 181-182, wherein R³ is hydrogen, substituted or unsubstituted C₁-C₄ alkyl, or substituted or unsubstituted phosphonyl.

184. The composition of any one of claims 181-183, wherein R³ is hydrogen, CH₃, or PO₃H₂.

185. The composition of any one of claims 181-184, wherein R⁸ is a water solubilizing group.

186. The composition of any one of claims 181-185, wherein R⁹ is a water solubilizing group.

187. The composition of any one of claims 181-186, wherein R¹⁰ is a water solubilizing group.

188. The composition of any one of claims 181-187, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

189. The composition of any one of claims 181-188, wherein R⁸, R⁹ and R¹⁰ are independently hydrogen, CH₃, C(O)CH₃, or PO₃H₂.

190. The composition of any one of claims 181-189, wherein R⁸, R⁹ and R¹⁰ are independently hydrogen, CH₃, or C(O)CH₃.

191. The composition of any one of claims 181-190, wherein the compound is of Formula VI-B: 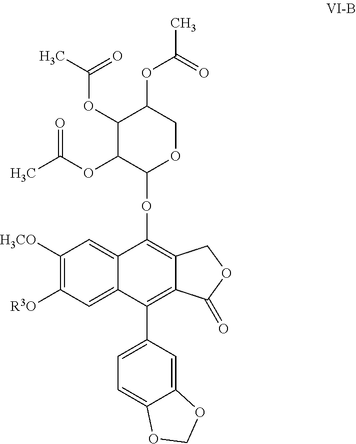
wherein
R³ is a water solubilizing group;
or a pharmaceutically acceptable salt or prodrug thereof.

192. The composition of claim 191, wherein R³ is a substituted or unsubstituted phosphonyl.

193. The composition of any one of claims 191-192, wherein the compound is of Formula VI-B-2: 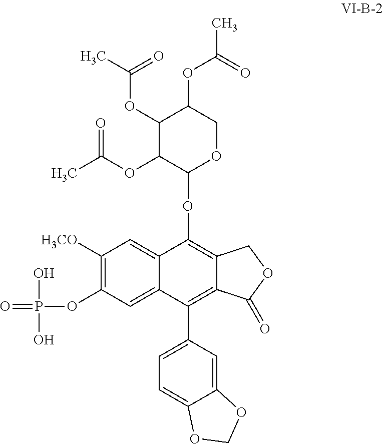 [SMILES: COc1cc2c(OC3OCC(OC(C)=O)C(OC(C)=O)C3OC(C)=O)c3c(c(-c4ccc5c(c4)OCO5)c2cc1OP(=O)(O)O)C(=O)OC3]
or a pharmaceutically acceptable salt or prodrug thereof.

194. The composition of any one of claims 181-190, wherein the compound is of Formula VI-C: 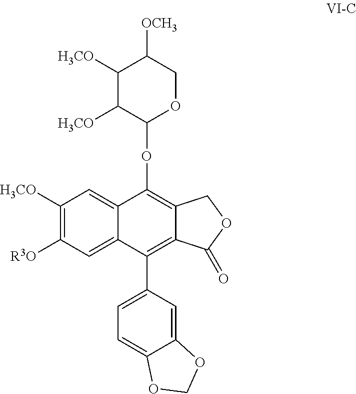
wherein
R³ is a water solubilizing group;
or a pharmaceutically acceptable salt or prodrug thereof.

195. The composition of claim 194, wherein R³ is a substituted or unsubstituted phosphonyl.

196. The composition of any one of claims 194-195, wherein the compound is of Formula VI-C-2: 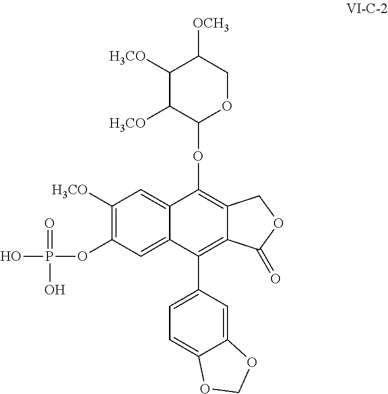 [SMILES: COc1cc2c(OC3OCC(OC)C(OC)C3OC)c3c(c(-c4ccc5c(c4)OCO5)c2cc1OP(=O)(O)O)C(=O)OC3]
or a pharmaceutically acceptable salt or prodrug thereof.

197. The composition of any one of claims 171-180, wherein the compounds are of Formula VI-D: 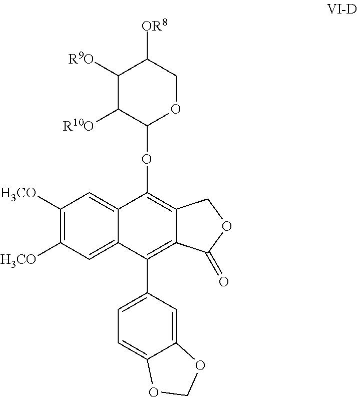
wherein
R⁸, R⁹ and R¹⁰ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
with the proviso that at least one of R⁸-R¹⁰ is a substituted or unsubstituted phosphonyl;
or a pharmaceutically acceptable salt or prodrug thereof.

198. The composition of claim 197, wherein R⁸ is a water solubilizing group.

199. The composition of any one of claims 197-198, wherein R⁹ is a water solubilizing group.

200. The composition of any one of claims 197-199, wherein R⁸ and R⁹ are independently hydrogen, substituted or unsubstituted C₁-C₆ alkyl, substituted or unsubstituted C₁-C₆ acyl, or substituted or unsubstituted phosphonyl.

201. The composition of any one of claims 197-200, wherein R⁸ and R⁹ are independently hydrogen, CH₃, C(O)CH₃, or PO₃H₂.

202. The composition of any one of claims 197-201, wherein R⁸ and R⁹ are independently hydrogen, CH₃, or C(O)CH₃.

203. The composition of any one of claims 197-202, wherein the compound is of Formula VI-E: 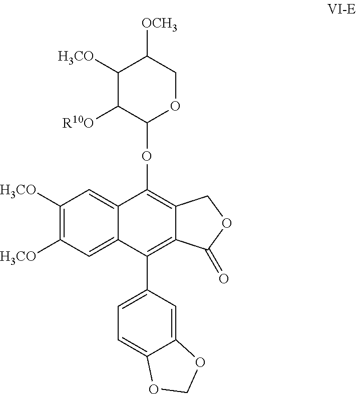
wherein
R¹⁰ is a water solubilizing group;
or a pharmaceutically acceptable salt or prodrug thereof.

204. The composition of claim 203, wherein R¹⁰ is a substituted or unsubstituted phosphonyl.

205. The composition of any one of claims 203-204, wherein the compound is of Formula VI-E-2: 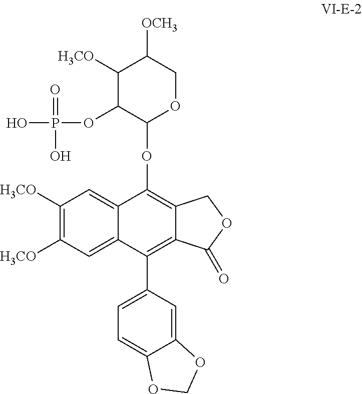 [SMILES: COc1cc2c(OC3OCC(OC)C(OC)C3OP(=O)(O)O)c3c(c(-c4ccc5c(c4)OCO5)c2cc1OC)C(=O)OC3]
or a pharmaceutically acceptable salt or prodrug thereof.

206. A method of treating or preventing cancer in a subject, comprising administering to the subject an effective amount of the composition of any one of claims 128-205.

207. The method of claim 206, wherein the cancer is selected from the group consisting of bladder, cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

208. The method of any one of claims 206-207, wherein the cancer is colon cancer.

209. The method of any one of claims 206-208, further comprising administering a second compound or composition, wherein the second compound or composition includes an anticancer agent,

210. The method of any one of claims 206-209, further comprising administering an effective amount of ionizing radiation to the subject.

211. A method of killing a tumor cell in a subject, comprising: contacting the tumor cell with an effiNtive amount of the composition of any one of claims 128-205.

212. The method of claim 211, further comprising contacting the tumor cell with a second compound or composition, wherein the second compound or composition includes an anticancer agent.

213. The method of any one of claims 211-212., wherein the tumor cell is a colon cancer

214. The method of any one of claims 211-213, further comprising irradiating the tumor cell with an effective amount of ionizing radiation.

215. A method of radiotherapy of a tumor, comprising:
contacting the tumor with an effiNtive amount of the composition of any one of claims 128-205; and
irradiating the tumor with an effective amount of ionizing radiation.

216. A method of treating or preventing cancer in a subject, comprising administering to the subject an effective amount composition comprising a compound of Formula I: 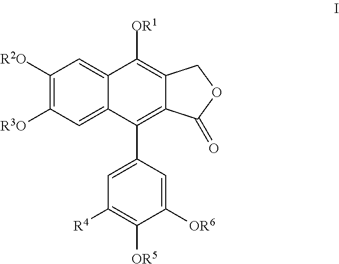
wherein
R¹ is hydrogen, halogen, formyl, substituted or unsubstituted C₁-C₆ alkyl, substituted or unsubstituted C₄-C₁₀ cycloalkyl, substituted or unsubstituted C₄-C₁₀ heterocycloalkyl, substituted or unsubstituted alkoxycarbonyl, hydroxycarbonyl, or substituted or unsubstituted acyl;
R² and R³ are independently hydrogen, halogen, substituted or unsubstituted substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R² and R³ taken together with the atoms to which they are attached form a substituted or unsubstituted 5 to 7 membered heterocyclic moiety;
R⁴ is hydrogen, hydroxy, halogen, nitro, cyano, formyl, substituted or unsubstituted alkyl, substituted or unsubstituted cycloalkyl, substituted or unsubstituted heterocycloalkyl, substituted or unsubstituted alkenyl, substituted or unsubstituted cycloalkenyl, substituted or unsubstituted heterocycloalkenyl., substituted or unsubstituted alkynyl, substituted or unsubstituted alkoxy, substituted or unsubstituted alkoxycarbonyl, hydroxycarbonyl, substituted or unsubstituted acyl, substituted or unsubstituted aryl, substituted or unsubstituted heteroaryl, substituted or unsubstituted amino, substituted or unsubstituted amido, substituted or unsubstituted carbamoyl, substituted or unsubstituted phosphonyl, substituted or unsubstituted silyl, substituted or unsubstituted sulfinyl, substituted or unsubstituted sulfonyl, substituted or unsubstituted sulfonamide, or substituted or unsubstituted thio;
R⁵ and R⁶ are independently hydrogen, halogen, substituted or unsubstituted alkyl, substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R⁵ and R⁶ taken together with the atoms to which they are attached form a substituted or unsubstituted 5 to 7 membered heterocyclic moiety;
or a pharmaceutically acceptable salt or prodrug thereof;
wherein the compound of Formula I is not a topoisomerase II inhibitor.

217. The method of claim 216, wherein R¹ is selected from:
H, CH₃, 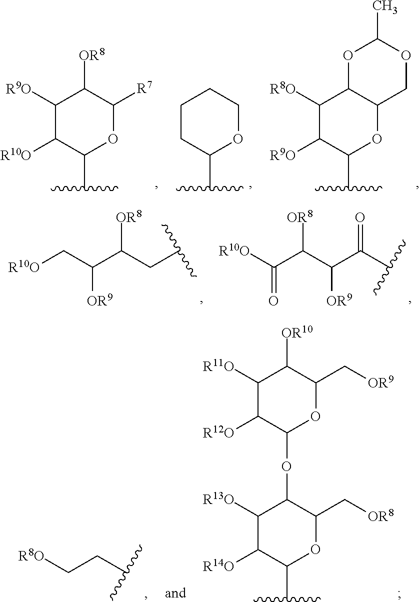
wherein, when present,
R⁷ is hydrogen, hydroxy, halogen, formyl, substituted or unsubstituted C₁-C₆ substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsitbstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted silyl, substituted or unsubstituted sulfinyl, substituted or unsubstituted sulfonyl, substituted or unsubstituted sulfonamide, or substituted or unsubstituted thio; and
R⁸, R⁹, R¹⁰, R¹¹, R¹², R¹³, and R¹⁴ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆carbamoyl, substituted or unsubstituted phosphonyl, substituted or unsubstituted sulfinyl, substituted or unsubstituted sulfonyl, substituted or unsubstituted sulfonamide, or substituted or unsubstituted thio.

218. The method of any one of claims 216-217, wherein R⁷ is hydrogen, substituted or unsubstituted C₁-C₆ alkyl, or substituted or unsubstituted C₁-C₆ acyl.

219. The method of any one of claims 216-218, wherein R⁷ is hydrogen, CH₂C(O)CH₃, or CH₂OH.

220. The method of any one of claims 216-219, wherein R⁸ is a water solubilizing group.

221. The method of any one of claims 216-220, wherein R⁹ is a water solubilizing group.

222. The method of any one of claims 216-221, wherein R¹⁰ is a water solubilizing group.

223. The method of any one of claims 216-222, wherein R¹¹ is a water solubilizing group.

224. The method of any one of claims 216-223, wherein R¹² is a water solubilizing group.

225. The method of any one of claims 216-224, wherein R¹³ is a water solubilizing group.

226. The method of any one of claims 216-225, wherein R¹⁴ is a water solubilizing group.

227. The method of any one of claims 216-226, wherein R⁸, R⁹, R¹⁰, R¹¹, R¹², R¹³, and R¹⁴ are independently hydrogen, substituted or unsubstituted C₁-C₆ alkyl, substituted or unsubstituted C₁-C₆ acyl, or substituted or unsubstituted phosphonyl.

228. The method of any one of claims 216-227, wherein R⁸, R⁹, R¹⁰, R¹¹, R¹², R¹³, and R¹⁴ are independently hydrogen, CH₃, C(O)CH₃, or PO₃H₂.

229. The method of any one of claims 216-228, wherein R⁸, R⁹, R¹⁰, R¹¹, R¹², R¹³, and R¹⁴ are independently hydrogen, CH₃, or C(O)CH₃.

230. The method of any one of claims 216-229, wherein R¹ is 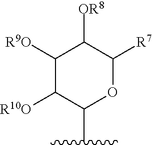
and
R⁷ is hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted substituted or unsubstituted sulfinyl, substituted or unsubstituted sulfonyl, substituted or unsubstituted sulfonamide, or substituted or unsubstituted thio; and
R⁸, R⁹, and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆carbamoyl, substituted or unsubstituted phosphonyl, substituted or unsubstituted sulfinyl, substituted or unsubstituted sulfonyl, substituted or unsubstituted sulfonamide, or substituted or unsubstituted thio.

231. The method of claim 230, wherein R⁷ is hydrogen, substituted or unsubstituted C₁-C₆ alkyl, or substituted or unsubstituted C₁-C₆ acyl.

232. The method of any one of claims 230-231, wherein R⁷ is hydrogen, CH₂C(O)CH₃, or CH₂OH.

233. The method of any one of claims 230-232, wherein R⁸ is a water solubilizing group.

234. The method of any one of claims 230-233, wherein R⁹ is a water solubilizing group.

235. The method of any one of claims 230-234, wherein R¹⁰ is a water solubilizing group.

236. The method of any one of claims 230-235, wherein R⁸, R⁹, and R¹⁰ are independently hydrogen, substituted or unsubstituted C₁-C₆ alkyl, substituted or unsubstituted C₁-C₆ acyl, or substituted or unsubstituted phosphonyl,

237. The method of any one of claims 230-236, wherein R⁸, R⁹, and R¹⁰ are independently hydrogen, CH₃, C(O)CH₃, or PO₃H₂.

238. The method of any one of claims 203-237, wherein R⁸, R⁹, and R¹⁰ are independently hydrogen, CH₃, or C(O)CH₃.

239. The method of any one of claims 216-238, wherein R¹ is

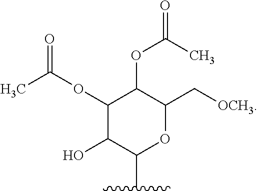 [SMILES: *C1OC(COC)C(OC(C)=O)C(OC(C)=O)C1O]

240. The method of any one of claims 216-239, wherein R² is a water solubilizing group.

241. The method of any one of claims 216-240, wherein R³ is a water solubilizing group.

242. The method of any one of claims 216-241, wherein R² and R³ are independently hydrogen, substituted or unsubstituted C₁-C₄ alkyl, or substituted or unsubstituted phosphonyl.

243. The method of any one of claims 216-242, wherein R² and R³ are independently hydrogen, CH₃, or PO₃H₂.

244. The method of any one of claims 216-243, wherein R⁴ is a water solubilizing group.

245. The method of any one of claims 216-244, wherein R⁴ is hydrogen, hydroxy, substituted or unsubstituted C₁-C₆ alkyl, substituted or unsubstituted C₁-C₆ alkoxy, substituted or unsubstituted C₁-C₆ acyl, or substituted or unsubstituted phosphonyl.

246. The method of any one of claims 216-245, wherein R⁴ is hydrogen.

247. The method of any one of claims 216-246, wherein R⁵ is a water solubilizing group.

248. The method of any one of claims 216-247, wherein R⁶ is a water solubilizing group.

249. The method of any one of claims 216-248, wherein R⁵ and R⁶ are independently hydrogen, substituted or unsubstituted C₁-C₄ alkyl, substituted or unsubstituted phosphonyl, or together with the atoms to which they are attached form a 5 membered heterocyclic group.

250. The method of any one of claims 216-249, wherein R⁵ and R⁶ are independently hydrogen, CH₃, PO₃H₂, or together with the atoms to which they are attached form a 5 membered heterocyclic group.

251. The method of any one of claims 216-250, wherein R⁵ and R⁶ together form a 5 membered heterocyclic group.

252. The method of any one of claims 216-251, wherein the compound of Formula I activates caspase-3.

253. A method of treating or preventing cancer in a subject, comprising administering to the subject an effective amount composition comprising a compound of Formula VI: 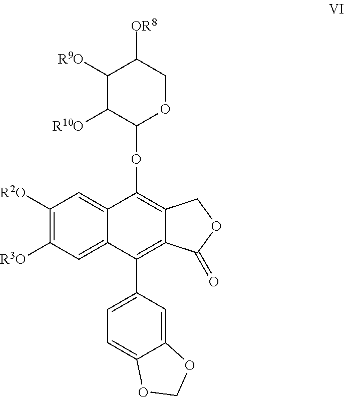
wherein
R² and R³ are independently hydrogen, halogen, substituted or unsubstituted C₁-C₄ alkyl, substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄ carbamoyl, substituted or unsubstituted phosphonyl, substituted or unsubstituted sulfinyl, substituted or unsubstituted sulfonyl, substituted or unsubstituted sulfonamide, or substituted or unsubstituted thio; or R² and R³ taken together with the atoms to which they are attached form a substituted or unsubstituted 5 to 7 membered heterocyclic moiety;
R⁸, R⁹ and R¹⁰ are independently hydrogen, halogen, substituted or unsubstituted C₁-C₆ alkyl, substituted or unsubstituted alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof;
wherein the compound of Formula VI is not a topoisomerase II inhibitor.

254. The method of claim 253, wherein R² is a water solubilizing group.

255. The method of any one of claims 253-254, wherein R³ is a water solubilizing group.

256. The method of any one of claims 253-255, wherein R² and R³ are independently hydrogen, substituted or unsubstituted C₁-C₄ alkyl, or substituted or unsubstituted phosphonyl.

257. The method of any one of claims 253-256, wherein R² and R³ are independently hydrogen, CH₃, or PO₃H₂.

258. The method of any one of claims 253-257, wherein R⁸ is a water solubilizing group.

259. The method of any one of claims 253-258, wherein R⁹ is a water solubilizing group.

260. The method of any one of claims 253-259, wherein R¹⁰ is a water solubilizing group.

261. The method of any one of claims 253-260, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

262. The method of any one of claims 253-261, wherein R⁸, R⁹ and R¹⁰ are independently hydrogen, CH₃, C(O)CH₃, or PO₃H₂.

263. The method of any one of claims 253-262, wherein the compound of Formula VI activates caspase-3.

264. The method of any one of claims 216-263, wherein the cancer is selected from the group consisting of bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer.

265. The method of any one of claims 216-264, wherein the cancer is colon cancer.

266. The method of any one of claims 216-265, further comprising administering a second compound or composition, wherein the second compound or composition includes an anticancer agent.

267. The method of any one of claims 216-266, further comprising administering an effective amount of ionizing radiation to the subject.

268. A method of killing a tumor cell in a subject, comprising administering to the subject an effective amount composition comprising a compound of Formula I: 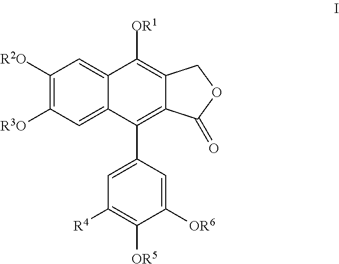
wherein
R¹ is hydrogen, halogen, formyl, substituted or unsubstituted C₁-C₆ alkyl, substituted or unsubstituted C₄-C₁₀ cycloalkyl, substituted or unsubstituted C₄-C₁₀ heterocycloalkyl, substituted or unsubstituted alkoxycarbonyl, hydroxycarbonyl, or substituted or unsubstituted acyl;
R² and R³ are independently hydrogen, halogen, substituted or unsubstituted substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R² and R³ taken together with the atoms to which they are attached form a substituted or unsubstituted 5 to 7 membered heterocyclic moiety;
R⁴ is hydrogen, hydroxy, halogen, nitro, cyano, formyl, substituted or unsubstituted alkyl, substituted or unsubstituted cycloalkyl, substituted or unsubstituted heterocycloalkyl, substituted or unsubstituted alkenyl, substituted or unsubstituted cycloalkenyl, substituted or unsubstituted heterocycloalkenyl, substituted or unsubstituted alkynyl, substituted or unsubstituted alkoxy, substituted or unsubstituted alkoxycarbonyl, hydroxycarbonyl, substituted or unsubstituted acyl, substituted or unsubstituted aryl, substituted or unsubstituted heteroaryl, substituted or unsubstituted amino, substituted or unsubstituted amido, substituted or unsubstituted carbamoyl, substituted or unsubstituted phosphonyl, substituted or unsubstituted sityl, substituted or unsubstituted sulfinyl, substituted or unsubstituted sulfonyl, substituted or unsubstituted sulfonamide, or substituted or unsubstituted thio;
R⁵ and R⁶ are independently hydrogen, halogen, substituted or unsubstituted alkyl, substituted or unsubstituted alkoxycarbonyl, hydroxycarbonyl, substituted or unsubstituted acyl, substituted or unsubstituted amino, substituted or unsubstituted amido, substituted or unsubstituted carbamoyl, substituted or unsubstituted phosphonyl, substituted or unsubstituted sulfinyl, substituted or unsubstituted sulfonyl, substituted or unsubstituted sulfonamide, substituted or unsubstituted thio, or R⁵ and R⁶ taken together with the atoms to which they are attached form a substituted or unsubstituted 5 to 7 membered heterocyclic moiety;
or a pharmaceutically acceptable salt or prodrug thereof;
wherein the compound of Formula I is not a topoisomerase II inhibitor.

269. The method of claim 268, wherein R¹ is selected from:
H, CH₃, 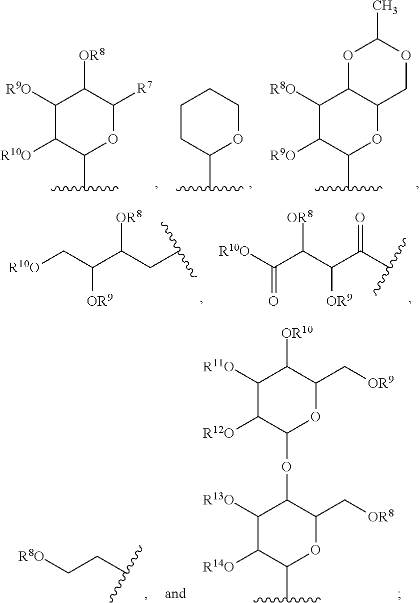
wherein, when present,
R⁷ is hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted silyl, substituted or unsubstituted sulfinyl, substituted or unsubstituted sulfonyl, substituted or unsubstituted sulfonamide, or substituted or unsubstituted thio; and
R⁸, R⁹, R¹⁰, R¹¹, R¹², R¹³, and R¹⁴ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio.

270. The method of any one of claims 268-269, wherein R⁷ is hydrogen, substituted or unsubstituted C₁-C₆ alkyl, or substituted or unsubstituted C₁-C₆ acyl.

271. The method of any one of claims 268-269, wherein R⁷ is hydrogen, CH₂C(O)CH₃, or CH₂OH.

272. The method of any one of claims 268-270, wherein R⁸ is a water solubilizing group.

273. The method of any one of claims 268-271, wherein R⁹ is a water solubilizing group.

274. The method of any one of claims 268-272, wherein R¹⁰ is a water solubilizing group.

275. The method of any one of claims 268-273, wherein R¹¹ is a water solubilizing group.

276. The method of any one of claims 268-274, wherein R¹² is a water solubilizing group.

277. The method of any one of claims 268-275, wherein R¹³ is a water solubilizing group.

278. The method of any one of claims 268-276, wherein R¹⁴ is a water solubilizing group.

279. The method of any one of claims 268-278, wherein R⁸, R⁹, R¹⁰, R¹¹, R¹², R¹³, and R¹⁴ are independently hydrogen, substituted or unsubstituted C₁-C₆ alkyl, substituted or unsubstituted C₁-C₆ acyl, or substituted or unsubstituted phosphonyl.

280. The method of any one of claims 268-279, wherein R⁸, R⁹, R¹⁰, R¹¹, R¹², R¹³, and R¹⁴ are independently hydrogen, CH₃, C(O)CH₃, or PO₃H₂.

281. The method of any one of claims 268-280, wherein R⁸, R⁹, R¹⁰, R¹¹, R¹², R¹³, and R¹⁴ are independently hydrogen, CH₃, or C(O)CH₃.

282. The method of any one of claims 268-281, wherein R¹ is 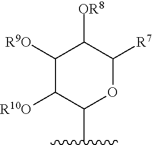
and
R⁷ is hydrogen, hydroxy, halogen, formyl, substituted or unsubstituted C₁-C₆ alkyl, substituted or unsubstituted C₂-C₆ alkenyl, substituted or unsubstituted C₂-C₆ alkynyl, substituted or unsubstituted C₁-C₆ alkoxy, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted substituted or unsubstituted sulfinyl, substituted or unsubstituted sulfonyl, substituted or unsubstituted sulfonamide, or substituted or unsubstituted thio; and
R⁸, R⁹, and R¹⁰ are independently hydrogen, halogen, substituted or unsubstituted C₁-C₆ alkyl, substituted or unsubstituted C₁-C₆ alkoxycarbonyl, hydroxycarbonyl, substituted or unsubstituted C₁-C₆ acyl, substituted or unsubstituted amino, substituted or unsubstituted amido, substituted or unsubstituted C₁-C₆ carbamoyl, substituted or unsubstituted phosphonyl, substituted or unsubstituted sulfinyl, substituted or unsubstituted sulfonyl, substituted or unsubstituted sulfonamide, or substituted or unsubstituted thio.

283. The method of claim 282, wherein R⁷ is hydrogen, substituted or unsubstituted C₁-C₆ alkyl, or substituted or unsubstituted C₁-C₆ acyl.

284. The method of any one of claims 282-283, wherein R⁷ is hydrogen, CH₂C(O)CH₃, or CH₂OH.

285. The method of any one of claims 282-284, wherein R⁸ is a water solubilizing group.

286. The method of any one of claims 282-285, wherein R⁹ is a water solubilizing group.

287. The method of any one of claims 282-286, wherein R¹⁰ is a water solubilizing group.

288. The method of any one of claims 282-287, wherein R⁸, R⁹, and R¹⁰ are independently hydrogen, substituted or unsubstituted C₁-C₆ alkyl, substituted or unsubstituted C₁-C₆ acyl, or substituted or unsubstituted phosphonyl.

289. The method of any one of claims 282-288, wherein R⁸, R⁹, and R¹⁰ are independently hydrogen, CH₃, C(O)CH₃, or PO₃H₂.

290. The method of any one of claims 282-289, wherein R⁸, R⁹, and R¹⁰, are independently hydrogen, CH₃, or C(O)CH₃.

291. The method of any one of claims 268-290, wherein R¹ is

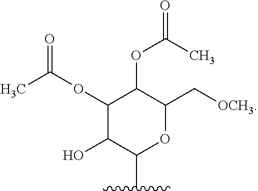 [SMILES: *C1OC(COC)C(OC(C)=O)C(OC(C)=O)C1O]

292. The method of any one of claims 268-291, wherein R² is a water solubilizing group.

293. The method of any one of claims 768-292, wherein R³ is a water solubilizing group.

294. The method of any one of claims 268-293, wherein R² and R³ are independently hydrogen, substituted or unsubstituted C₁-C₄ alkyl, or substituted or unsubstituted phosphonyl.

295. The method of any one of claims 268-294, wherein R² and R³ are independently hydrogen, CH₃, or PO₃H₂.

296. The method of any one of claims 268-295, wherein R⁴ is a water solubilizing group.

297. The method of any one of claims 268-296, wherein R⁴ is hydrogen, hydroxy, substituted or unsubstituted C₁-C₆ alkyl, substituted or unsubstituted. C₁-C₆ alkoxy, substituted or unsubstituted C₁-C₆ acyl, or substituted or unsubstituted phosphonyl.

298. The method of any one of claims 268-297, wherein R⁴ is hydrogen.

299. The method of any one of claims 768-298, wherein R⁵ is a water solubilizing group.

300. The method of any one of claims 268-299, wherein R⁶ is a water solubilizing group.

301. The method of any one of claims 268-300, wherein R⁵ and R⁶ are independently hydrogen, substituted or unsubstituted C₁-C₄ alkyl, substituted or unsubstituted phosphonyl, or together with the atoms to which they are attached form a 5 membered heterocyclic group.

302. The method of any one of claims 268-301, wherein R⁵ and R⁶ are independently hydrogen, CH₃, PO₃H₂, or together with the atoms to which they are attached form a 5 membered heterocyclic group.

303. The method of any one of claims 268-302, wherein R⁵ and R⁶ together form a 5 membered heterocyclic group.

304. The method of any one of claims 268-303, wherein the compound of Formula I activates caspase-3.

305. A method of treating or preventing cancer in a subject, comprising administering to the subject an effective amount composition comprising a compound of Formula VI: 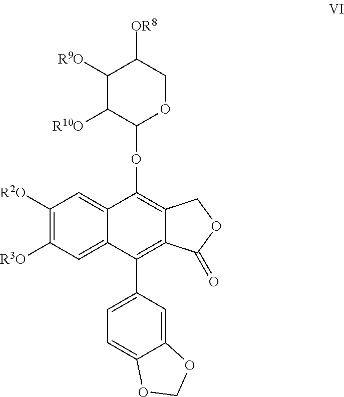
wherein
R² and R³ are independently hydrogen, halogen, substituted or unsubstituted C₁-C₄ substituted or unsubstituted C₁-C₄ alkoxycarbonyl, hydroxycarbonyl, substituted or unsubstituted C₁-C₄ acyl, substituted or unsubstituted amino, substituted or unsubstituted amido, substituted or unsubstituted C₁-C₄carbamoyl, substituted or unsubstituted phosphonyl substituted or unsubstituted sulfinyl substituted or unsubstituted sulfonyl, substituted or unsubstituted sulfonamide, or substituted or unsubstituted thio; or R² and R³ taken together with the atoms to which they are attached form a substituted or unsubstituted 5 to 7 membered heterocyclic moiety;
R⁸, R⁹ and R¹⁰ are independently hydrogen, halogen, substituted or unsubstituted C6 alkyl, substituted or unsubstituted C₁-C₆ alkoxycarbonyl, hydroxycarbonyl substituted or unsubstituted C₁-C₆ acyl, substituted or unsubstituted amino, substituted or unsubstituted amido, substituted or unsubstituted C₁-C₆carbamoyl, substituted or unsubstituted phosphonyl, substituted or unsubstituted sulfinyl, substituted or unsubstituted sulfonyl, substituted or unsubstituted sulfonamide, or substituted or unsubstituted thio;
or a pharmaceutically acceptable salt or prodrug thereof;
wherein the compound of Formula VI is not a topoisomerase II inhibitor.

306. The method of claim 305, wherein R² is a water solubilizing group.

307. The method of any one of claims 305-306, wherein R³ is a water solubilizing group.

308. The method of any one of claims 305-307, wherein R² and R³ are independently hydrogen, substituted or unsubstituted C₁-C₄ alkyl, or substituted or unsubstituted phosphonyl.

309. The method of any one of claims 305-308, wherein R² and R³ are independently hydrogen, CH₃, or PO₃H₂.

310. The method of any one of claims 305-309, wherein R⁸ is a water solubilizing group.

311. The method of any one of claims 305-310, wherein R⁹ is a water solubilizing group.

312. The method of any one of claims 305-311, wherein R¹⁰ is a water solubilizing group.

313. The method of any one of claims 305-312, wherein R⁸, R⁹ and R¹⁰ are independently hydrogen, substituted or unsubstituted C₁-C₆ alkyl, substituted or unsubstituted C₁-C₆ acyl, or substituted or unsubstituted phosphonyl.

314. The method of any one of claims 305-313, wherein R⁸, R⁹ and R¹⁰ are independently hydrogen, CH₃, C(O)CH₃, or PO₃H₂.

315. The method of any one of claims 305-314, wherein the compound of Formula VI activates caspase-3.

316. The method of any one of claims 268-315, further comprising contacting the tumor cell with a second compound or composition, wherein the second compound or composition includes an anticancer agent.

317. The method of any one of claims 268-316, wherein t le tumor cell is a colon cancer cell.

318. The method of any one of claims 268-317, further comprising irradiating the tumor cell with an effective amount of ionizing radiation.

319. A method of stimulating a human natural killer cell in a subject, comprising:
administering to the subject an effective amount of the composition of any one of claims 128-205.
